(19) United States
(12) Patent Application Publication
(10) Pub. No.: US 2025/0213151 A1
(43) Pub. Date: Jul. 3, 2025

(54) SYSTEMS AND METHODS FOR PROVIDING THERAPY MANAGEMENT GUIDANCE RELATED TO LIVER DISEASE DECOMPENSATION TO USERS WITH LIVER DISEASE

(71) Applicant: Dexcom, Inc., San Diego, CA (US)

(72) Inventors: Matthew L. JOHNSON, San Diego, CA (US); Qi AN, San Diego, CA (US); John PADERI, San Diego, CA (US); Rush BARTLETT, San Diego, CA (US)

(21) Appl. No.: 18/999,275

(22) Filed: Dec. 23, 2024

Related U.S. Application Data
(60) Provisional application No. 63/615,754, filed on Dec. 28, 2023.

(51) Int. Cl. A61B 5/1473; A61B 5/00; A61B 5/02; A61B 5/145; A61B 5/1495; A61B 5/20; G16H 20/00

(52) U.S. Cl. CPC A61B 5/1473; A61B 5/02042; A61B 5/14532; A61B 5/14546; A61B 5/1495; A61B 5/201; A61B 5/4064; A61B 5/412; A61B 5/4244; A61B 5/7225; A61B 5/7264; A61B 5/7275; G16H 20/00; A61B 2560/0223; A61B 2560/0462

(57) ABSTRACT

In certain embodiments, a continuous analyte monitoring system is provided. The continuous analyte system includes one or more continuous analyte sensors configured to generate one or more sensor signals indicative of one or more analyte levels, and at least one sensor electronics module. The at least one sensor electronics module includes an analog to digital converter configured to receive the sensor signal and convert the sensor current into digital signals. The continuous analyte monitoring system further includes a wireless transceiver configured to transmit the digital signals to a wireless communications device, one or more memories comprising executable instructions, and one or more processors. The one or more processors are configured to execute the executable instructions to convert the digital signals to a set of estimated analyte measurements indicative of the true analyte levels of the user, and generate therapy management guidance based on the estimated analyte measurements.

### CROSS-REFERENCE TO RELATED APPLICATIONS

This application claims priority to and benefit of U.S. Provisional Application No. 63/615,754, filed Dec. 28, 2023, which is hereby assigned to the assignee hereof and hereby expressly incorporated by reference in their entirety as if fully set forth below and for all applicable purposes.

### INTRODUCTION

Generally, diagnosis, staging, and treatment liver disease requires a combination of a variety of screening tools and/or procedures and investigation of specific symptoms of a user. As such, due to the difficulty in diagnosing and staging liver disease based on different combinations of symptoms, liver disease often remains undiagnosed and/or unmonitored until the disease becomes severe (e.g., when very little liver function remains). Additionally, liver disease may be reversible up until the user develops cirrhosis (e.g., severe liver disease), so early diagnosis may allow for improved liver disease outcomes.

Although some non-invasive screening tools, such as Fibroscan (an imaging based estimate of liver stiffness), metabolic assay panels (a blood test to test various liver analytes), and point-of-care analyte tests have become available, these tools are not always as reliable, timely, and/or accurate in confirming a liver disease diagnosis and monitoring progression of liver disease over time. Therefore, liver biopsies have become the gold standard for confirmatory diagnosis of liver disease; although biopsies are not widely used due to the invasiveness and cost of the procedure. Additionally, biopsies provide information on the structural aspects of the liver, which may not be as useful for determining the metabolic functional performance and, therefore, liver health and/or disease state of the liver cellular tissue.

In addition, the biopsies and screening tools discussed above are point-in-time diagnostic methods and do not provide insight into the health and functioning of the liver over time. A functional test at a single point in time may be of limited utility in monitoring an actively dynamic organ such as the liver.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example therapy management system used in connection with implementing embodiments of the present disclosure.

FIG. 2 is a diagram conceptually illustrating an example continuous analyte monitoring system including example continuous analyte sensor(s) with sensor electronics, according to certain embodiments of the present disclosure.

FIG. 3 illustrates example inputs and example metrics that are calculated based on the inputs for use by the therapy management system of FIG. 1, according to certain embodiments of the present disclosure.

FIG. 4 describes an example method for providing liver disease therapy management using an analyte monitoring system configured to measure at least potassium levels, according to certain embodiments of the present disclosure.

FIG. 5 describes an example method for providing liver disease therapy management using an analyte monitoring system configured to measure at least lactate levels, according to certain embodiments of the present disclosure.

FIG. 6 describes an example method for providing liver disease therapy management using an analyte monitoring system configured to measure at least ammonia levels, according to certain embodiments of the present disclosure.

FIG. 7 describes an example method for providing liver disease therapy management using an analyte monitoring system configured to measure at least sodium levels, according to certain embodiments of the present disclosure.

FIG. 8 describes an example method for providing liver disease therapy management using an analyte monitoring system configured to measure at least glucose levels, according to certain embodiments of the present disclosure.

FIG. 9 is a flow diagram depicting a method for providing liver disease therapy management, according to certain embodiments of the present disclosure.

FIG. 10 is a flow diagram depicting a method for training machine learning models to predict a user's disease state and/or health complications related to liver disease, and provide recommendations to a user based on the disease state, according to certain embodiments of the present disclosure.

FIG. 11 is a block diagram depicting a computing device configured to perform the operations of FIGS. 4-8, according to certain embodiments of the present disclosure.

FIGS. 12A-12B depict exemplary enzyme domain configurations for a continuous multi-analyte sensor, according to certain embodiments of the present disclosure.

FIGS. 12C-12D depict exemplary enzyme domain configurations for a continuous multi-analyte sensor, according to certain embodiments of the present disclosure.

FIG. 12E depicts an exemplary enzyme domain configuration for a continuous multi-analyte sensor, according to certain embodiments of the present disclosure.

FIGS. 13A-13B depict exemplary dual electrode enzyme domain configurations for a continuous multi-analyte sensor, according to certain embodiments of the present disclosure.

FIGS. 13C-13D depict exemplary dual electrode enzyme domain configurations for a continuous multi-analyte sensor, according to certain embodiments of the present disclosure.

FIG. 13E depicts an exemplary dual electrode configuration for a continuous multi-analyte sensor, according to certain embodiments of the present disclosure.

FIG. 14A depicts an exemplary enzyme domain configuration for a continuous multi-analyte sensor, according to certain embodiments of the present disclosure.

FIGS. 14B-14C depict alternative exemplary enzyme domain configurations for a continuous multi-analyte sensor, according to certain embodiments of the present disclosure.

FIG. 15 depicts an exemplary enzyme domain configuration for a continuous multi-analyte sensor, according to certain embodiments of the present disclosure.

FIGS. 16A-16D depict alternative views of exemplary dual electrode enzyme domain configurations G1-G4 for a continuous multi-analyte sensor, according to certain embodiments of the present disclosure.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Liver disease is not easily detectable. In particular, the liver is sometimes referred to as a silent organ as, even when liver failure occurs, the symptoms often go unnoticed. In some cases, once symptoms become apparent, the liver disease may have already reached an advanced stage (e.g., decompensated liver disease). Accordingly, early liver disease diagnosis and staging is vital to effectively treat, and in some cases, reverse the disease.

Physicians use information from a patient's history, physical examination, laboratory findings, and other diagnostic tests to diagnose and stage liver disease to prescribe appropriate treatment. For example, liver function tests may be used by physicians to screen for liver infection, monitor the progression of liver disease, assess the effectiveness of different treatments for liver disease, and monitor the possible side effects of medication to a patient's liver, to name a few. Liver function tests check the levels of certain enzymes and proteins in a patient's blood. Levels that are higher or lower than normal can, in some cases, indicate liver problems.

However, such conventional liver disease diagnostic and staging methods face many challenges with respect to efficiency, accuracy, and delay in providing liver diagnosis for treatment decision-making. For example, it can be very difficult to determine which particular diagnosis is indicated by a particular combination of symptoms, especially if symptoms are nonspecific, such as fatigue. Liver disease may also present atypically, with an unusual and unexpected constellation of symptoms.

Currently, the standard of care for definitive diagnosis of liver disease is a liver biopsy. A liver biopsy is a non-scalable and invasive way to diagnose liver disease. Accordingly, making an accurate diagnosis can prove to be particularly challenging for physicians. Further, when a patient seeks health care, there is an iterative process of information gathering, information integration and interpretation, and determining a working diagnosis, and throughout the diagnostic process, there is an ongoing assessment of whether sufficient information has been collected. If a physician is not satisfied that the necessary information has been collected to explain the patient's health problem or accurately diagnose the patient with liver disease, or that the information available is not consistent with a liver disease diagnosis, then the process of information gathering, information integration and interpretation, and developing a working diagnosis continues. Accordingly, diagnosis of the liver disease may be delayed and therefore, the patient's liver disease remains unmonitored. In some cases, lack of liver disease diagnosis and monitoring may contribute to a patient experiencing worsening symptoms, a decline in overall health, and even death given the time-dependent nature of many diseases, including liver disease and, specifically, decompensated liver disease.

Further, existing technologies, such as point of care (POC) devices, have been introduced to enable timely assessment of patients with, or at risk, of liver disease. One such POC device may include a portable, self-monitoring analyte monitor which typically requires the user to prick his or her finger to give a single standalone reading indicative of his or her analyte levels for diagnosing liver health. Thus far, POC devices have almost exclusively included point-in-time devices-devices that can analyze a patient to give a single standalone reading. As such, existing devices suffer from a technical problem of failing to continuously (and/or semi-continuously and/or periodically) monitor the concentration of changing analytes to give a continuous (and/or semi-continuous and/or periodic) readout. Such continuous monitoring of analytes is advantageous in diagnosing, staging, and monitoring progression of liver disease of a patient given the continuous measurements provide continuously up to date measurements as well as information on the trend and rate of analyte change over a continuous period.

Continuous measurements as proposed herein, provide a more accurate indication of liver metabolic function and liver disease progression over time as compared to a single point in time reading of a single analyte. A single point in time reading may be influenced by a patient's activity, such as exercise or diet changes near or during the point in time. Further, lactate point-in-time readings may be further influenced by sweat and/or dirt which may be present on the user's skin.

Additionally, imaging techniques that determine structural aspects of the liver do not provide information on the metabolic functional performance of liver cellular tissue. Measuring analytes (e.g., ammonia, glucose, sodium, potassium, and/or lactate) in a continuous readout as proposed herein may increase understanding of metabolic function of the liver to determine liver function, monitor the progression of liver disease over time (e.g., monitor for the development of decompensated liver disease), and/or diagnose or monitor various health complications related to worsening liver disease. Such information may also be used to make more informed decisions in the assessment of liver health, treatment of liver disease, and/or treatment of one or more health complications related to liver disease. As a result of this technical problem, monitoring the progression of liver disease over time is not technically possible, which, in some cases, might prove to be life threatening for a user with liver disease.

Accordingly, certain embodiments described herein provide a technical solution to the technical problem described above by providing a continuous analyte monitoring system, including, at least one of a continuous ammonia sensor, a continuous lactate sensor, a continuous potassium sensor, a continuous sodium sensor, or a continuous glucose sensor for use in staging, and/or monitoring liver disease and health complications related to and/or indicative of liver disease (e.g., hepatic encephalopathy, sepsis, variceal bleeding, ascites, infection, etc.). As used herein, the term “continuous” analyte monitoring provides a data stream in a continuous manner. In some examples, a data stream of analyte information is provided in a fully continuous, semi-continuous or periodic manner. In certain embodiments, analyte data is continuous when it is provided in a frequency that includes regular automatic measurements. The analyte data and measurements are provided in a continuous stream and present the current measured analyte state of the user over the time period of measurement. In one example, the analyte data is measured in the interstitial fluid and processed and calibrated to generate data indicative of current blood analyte levels.

Certain embodiments described herein also provide a therapy management system configured to use analyte data generated by the continuous analyte monitoring system described herein to provide therapy related to worsening liver disease, the development of decompensated liver disease, and/or the development of health complications related to liver disease. As described herein, liver disease therapy management can include therapy management related to worsening liver disease, the progression of liver disease from a compensated to a decompensated stage, and/or the development of, or risk of developing health complications related to worsening liver disease.

The present disclosure relates generally to methods and systems for continuously monitoring analyte data, including one of at least ammonia, lactate, potassium, sodium, or glucose levels, and/or non-analyte data to provide liver disease therapy management. For example, aspects of the present disclosure utilize analyte data as well as non-analyte data of a user to determine progression of liver disease, monitor for the development and/or progression of health complications related to worsening liver disease, and provide user-specific feedback (e.g., regarding medical intervention, medication recommendations, and/or lifestyle changes (e.g., limit consumption of alcohol, maintain a specific exercise regime, etc.)), and to monitor and adjust the therapy to prevent the progression and/or development of liver disease and/or health complications.

Determining risk of developing decompensated liver disease (e.g., through the development of health complications related to liver disease) and/or determining worsening liver disease and using real time continuous analyte information provides for therapy that is provided more frequently and earlier than otherwise possible with a biopsy or current screening procedures. This allows for better outcomes for users with liver disease, including treating early stage liver disease prior to the user developing decompensated liver disease, and lower the risk of developing health complications related to liver disease. In certain embodiments herein, potassium, sodium, lactate, ammonia, or glucose levels and metrics are utilized by non-invasive methods to provide liver disease therapy management. When liver health is monitored in a continuous manner and risk indicators are identified and monitored from an early stage in real time using analyte monitoring, real time feedback can be provided to the user and clinicians and caregivers to help prevent the disease or the progression thereof and prevent and/or treat health complications related to worsening liver disease. In some examples, recommendations include medical intervention, prescription medications, lifestyle changes, alcohol consumption recommendations, and/or exercise recommendations that can cause regression of liver disease, prevent progression of liver disease, cause regression of health complications related to liver disease, and/or prevent health complications related to liver disease.

As used herein, the term “continuous” analyte monitoring refers to monitoring one or more analytes in a fully continuous, semi-continuous, or periodic manner, which results in a data stream of analyte values over time without requiring user intervention (e.g., repeated finger sticks). A data stream of analyte values over time is what allows for meaningful data and insight to be derived using the algorithms described herein for determining a risk or presence of liver disease, determining the progression of liver disease, and providing patient-specific therapy management guidance (e.g., meal, exercise, liver disease stage information, and/or lifestyle recommendations) to prevent the progression and/or development of liver disease and/or other health complications. In other words, single point-in-time measurements collected as a result of a patient visiting their health care professional every few months, or using point of care meters, results in sporadic data points (e.g., that are, at best, hours, days or months apart in timing) that cannot form the basis of any meaningful data or insight to be derived. As such, without the continuous analyte monitoring system of the embodiments herein, it is simply impossible to determine through pattern analysis and continuously a risk or presence of liver disease, determine the progression of liver disease, as well as continuously provide therapy management recommendations (e.g., meal, exercise, liver disease stage information, and/or lifestyle recommendations) to prevent the progression and/or development of liver disease, as described herein.

The data stream of analyte values collected over time, with the continuous analyte monitoring system presented herein, include real-time analyte values (values representative of the current concentration of analyte values in the body), which allows for deriving meaningful data and insight in real-time using the systems and algorithms described herein. The derived real-time data and insight in turn allows for assessing the risk of disglycemia due to liver disease that can indicate the presence of such disease and/or progression of such disease, as well as real-time therapy management recommendations. Real time analyte values herein refer to analyte values that become available and actionable within seconds or minutes of being produced as a result of at least one sensor electronics module of the continuous analyte monitoring system (1) converting sensor current(s) (i.e., analog electrical signals) generated by the continuous analyte sensor(s) into sensor count values, (2) processing the count values (e.g., by filtering, smoothing, calibration or some combination thereof) to generate at least glucose concentration values using calibration techniques described herein to account for the sensitivity of the continuous analyte sensor(s), and (3) transmitting measured glucose concentration data, including glucose concentration values, to a display device via wireless connection.

For example, the at least one sensor electronics module can be configured to sample the analog electrical signals at a particular sampling period (or rate), such as every 1 second (1 Hz), 5 seconds, 10 seconds, 30 seconds, 1 minute, 3 minutes, 5 minutes, etc., and to transmit the measured glucose concentration data to a display device at a particular transmission period (or rate), which can be the same as (or longer than) the sampling period, such as every 1 minute (0.016 Hz), 5 minutes, 10 minutes, 15 minutes etc.

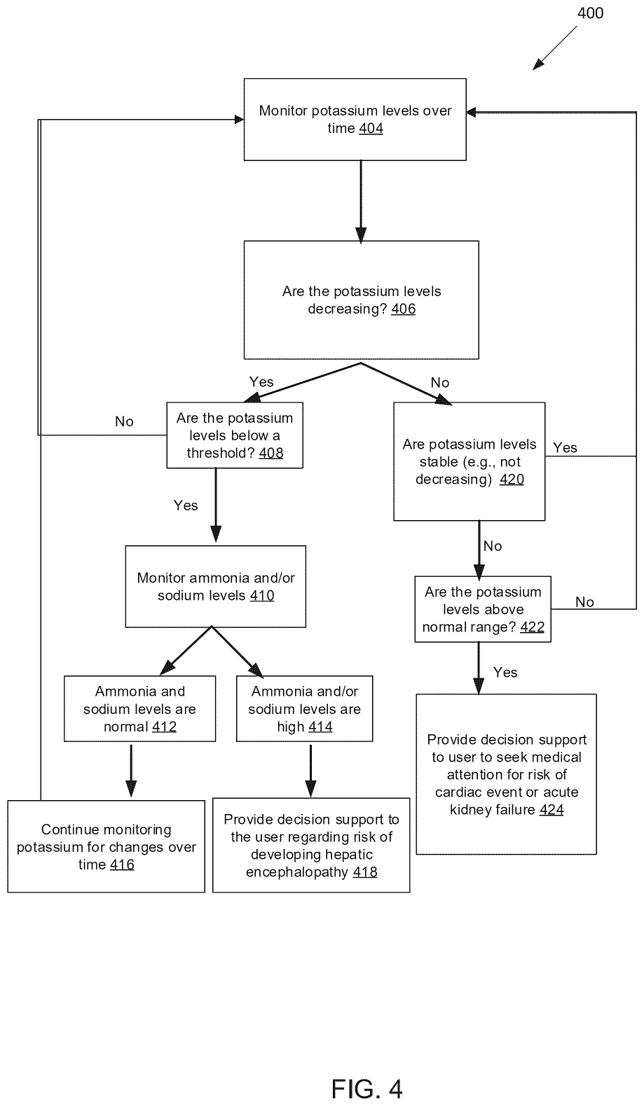

The real-time analyte data that is continuously generated by the continuous analyte monitoring system described herein, and therefore, allows the therapy management system herein to perform any or all of the functions to: determine a risk or presence of liver disease, determine the progression of liver disease, as well as provide therapy management recommendations, in real-time, which is technically impossible to perform using existing or conventional techniques or systems. Further, because of the real-time nature of this data, it is also humanly impossible to continuously process a real-time data stream of analyte values over time to derive meaningful data and insight using the algorithms and systems described herein for determining a risk or presence of liver disease, determining the progression of liver disease, as well as providing therapy management recommendations. In other words, deriving meaningful data and insight from a stream of real-time data that is continuously generated, processed, calibrated, and analyzed, using the algorithms and systems described herein, is not a task that can be mentally performed. For example, executing the algorithm described in relation to FIG. 4 in real-time and on a continuous basis, which would involve using a stream of real-time data that is continuously generated by a continuous analyte monitoring system worn by a host and/or significantly large amount of population data (e.g., hundreds or thousands of data points for each one of thousands or millions of users in the user population) is not a task that can be mentally performed, especially in real-time at times.

Further, the real-time data generated is not merely data, but data that is processed in a particular manner to allow for use in algorithms that require a certain type of data. Each analyte sensor system that is manufactured by a sensor manufacturer might perform slightly different. As such, there might be inconsistencies between sensors and the measurements they generate once in use. Accordingly, certain embodiments herein are directed to determining the performance of an analyte sensor system during a manufacturing calibration process (in vitro), which includes quantifying certain sensor operating parameters, such as a calibration slope (also known as calibration sensitivity), a calibration baseline, etc.

Generally, calibration sensitivity refers to the amount of electrical current produced by an analyte sensor of an analyte sensor system when immersed in a predetermined amount of a measured analyte. The amount of electrical current can be expressed in units of picoAmps (pA) or counts. The amount of measured analyte can be expressed as a concentration level in units of milligrams per deciliter (mg/dL), and the calibration sensitivity can be expressed in units of pA/(mg/dL) or counts/(mg/dL). The calibration baseline refers to the amount of electrical current produced by the analyte sensor when no analyte is detected, and can be expressed in units of pA or counts.

The calibration sensitivity, calibration baseline, and other information related to the sensitivity profile for the analyte sensor system can be programmed into the sensor electronics module of the analyte sensor system during the manufacturing process, and then used to convert the analyte sensor electrical signals into measured analyte concentration levels. For example, the calibration slope (calibration sensitivity) can be used to predict an initial in vivo sensitivity (M₀) and a final in vivo sensitivity (Mf), which are programmed into the sensor electronics module and used to convert the analyte sensor electrical signals into measured analyte concentration levels.

In certain embodiments, during in vivo use, the sensor electronics module of an analyte sensor system samples the analog electrical signals produced by the analyte sensor to generate analyte sensor count values, and then determines the measured analyte concentration levels based on the analyte sensor count values, the initial in vivo sensitivity (M₀), and the final in vivo sensitivity (M_{f}). For example, measured analyte concentration levels can be determined using a sensitivity function M(t) that is based on the initial in vivo sensitivity (M₀) and the final in vivo sensitivity (M_{f}). The sensitivity function M(t) can be expressed in several different ways, such as a simple correction factor that is not dependent on elapsed time (tᵢ) of in vivo use, a linear relationship between sensitivity and time (tᵢ), an exponential relationship between sensitivity and time (tᵢ), etc. Equation 1 presents one technique for determining a measured analyte concentration level (ACL) from an analyte sensor count value (count) at a time tᵢ:

$$ACL = count / M ( {t}_{i} ) \qquad Eq . 1$$

A calibration baseline (baseline) can also be used to determine a measured analyte concentration level (ACL) from an analyte sensor count value (count) at a time tᵢ, and Equation 2 presents one technique:

$$ACL = ( count - baseline ) / M ( ti ) \qquad Eq . 2$$

In addition, to allow for processing of the data in a distributed manner, various communication mechanisms must be used to transmit the data in processed or unprocessed format across multiple devices having various processing capabilities. Due to the sensitivity of health related data, as well as the danger to a user from any external malicious attempts, such transmissions must be performed in a secure and efficient manner. Example techniques for such data transmission and communication are described herein. But other transmission techniques can similarly be used.

Example Therapy Management System Including an Example Analyte Sensor for Determining Worsening Liver Disease and/or Health Complications Related to Liver Disease

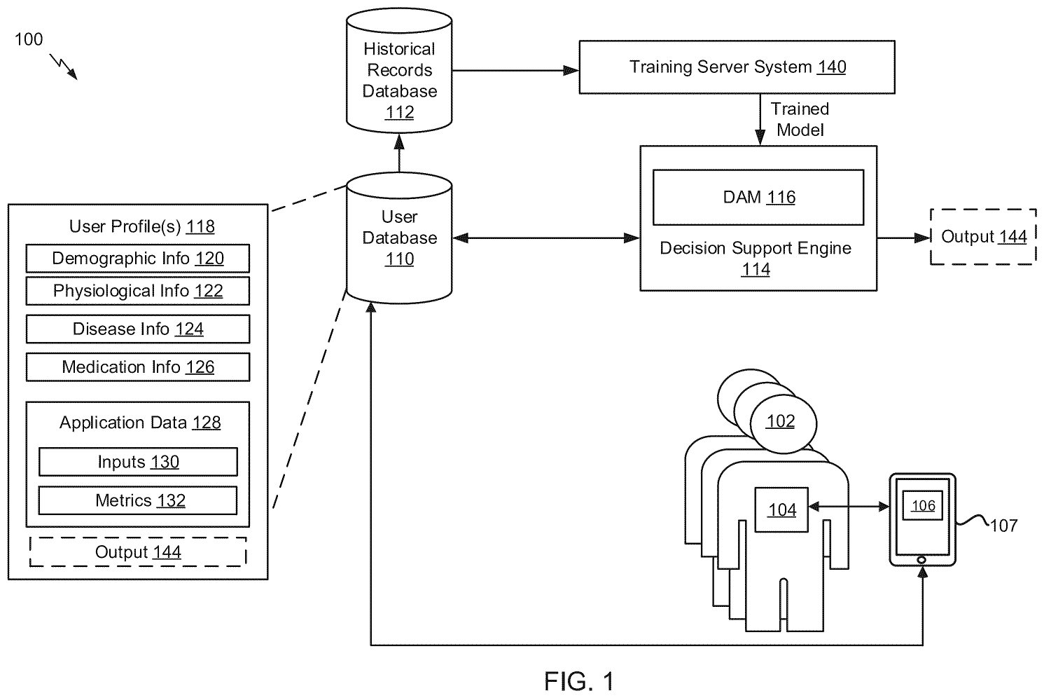

FIG. 1 illustrates an example therapy management system 100 for providing liver disease therapy management related to liver disease of users 102 (individually referred to herein as a user and collectively referred to herein as users), using a continuous analyte monitoring system 104 configured to continuously measure at least one of potassium, sodium, lactate, ammonia, or glucose levels. A user, in certain embodiments, is a user with liver disease, a user with one or more health complications related to liver disease, and/or a healthy user (e.g., a user not diagnosed with liver disease and/or one or more health complications related to liver disease), for example.

In certain embodiments, system 100 includes continuous analyte monitoring system 104, a display device 107 that executes application 106, a therapy management engine 114, a user database 110, a historical records database 112, a training system 140, and a therapy management engine 114, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. Analytes for measurement by the devices and methods can include, but may not be limited to, potassium, glucose, endogenous insulin, acarboxyprothrombin; acylcarnitine; exogenous insulin; adenine phosphoribosyl transferase; adenosine deaminase; albumin; albumin-creatinine ratio; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-peptide; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; creatinine; cyclosporin A; cystatin C; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; electrolytes; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; pyruvate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; proteinuria; pH; quinine; reverse tri-iodothyronine (rT3); selenium; serum pancreatic lipase; sisomicin; somatomedin C; specific antibodies recognizing any one or more of the following that can include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, IgE (atopic disease), influenza virus, Leishmania donovani, leptospira, measles/mumps/rubella, melatonin, Mycobacterium leprae, Mycoplasma pneumoniae, Myoglobin, Onchocerca volvulus, parainfluenza virus, Plasmodium falciparum, poliovirus, Pseudomonas aeruginosa, pro-C3, pro-C6, alpha-fetoprotein (AFP), IL-6, TNF-alpha, soluble urokinase plasminogen activator receptor (suPAR), malondialdehyde (MDA), 8-hydroxy-2′-deoxyguanosine (8-OHdG), bilirubin, cytokeratin-18 fragments respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones (e.g., insulin) naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include potassium, lactate, ammonia, sodium, and glucose, in some cases other analytes listed above can also be considered if enabled by the disclosure or otherwise known to a person skilled in the art.

In certain embodiments, continuous analyte monitoring system 104 is configured to continuously measure one or more analytes and transmit the analyte measurements to an electric medical records (EMR) system (not shown in FIG. 1). An EMR system is a software platform which allows for the electronic entry, storage, and maintenance of digital medical data. An EMR system is generally used throughout hospitals and/or other caregiver facilities to document clinical information on users over long periods. EMR systems organize and present data in ways that assist clinicians with, for example, interpreting health conditions and providing ongoing care, scheduling, billing, and follow up. Data contained in an EMR system can also be used to create reports for clinical care and/or disease management for a user. In certain embodiments, the EMR is in communication with therapy management engine 114 (e.g., via a network) for performing the techniques described herein. In particular, as described herein, therapy management engine 114 can obtain data associated with a user, use the obtained data as input into one or more trained model(s), and output a prediction. In some cases, the EMR provides the data to therapy management engine 114 to be used as input into the one or more models. Further, in some cases, therapy management engine 114, after making a prediction, provides the output prediction to the EMR.

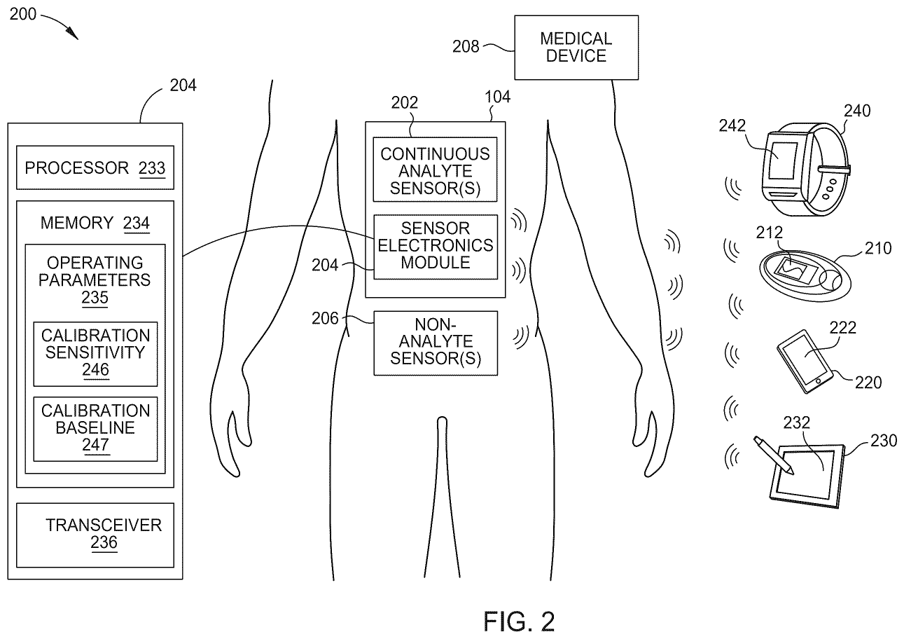

In certain embodiments, continuous analyte monitoring system 104 is configured to continuously measure one or more analytes and transmit the analyte measurements to display device 107 for use by application 106. In some embodiments, continuous analyte monitoring system 104 transmits the analyte measurements to display device 107 through a wireless connection (e.g., Bluetooth (e.g., including Bluetooth Low Energy (BLE)) connection, WiFi connection, local area network connection, cellular network connection, etc.). In certain embodiments, display device 107 is a smart phone. However, in certain other embodiments, display device 107 instead is any other type of computing device such as a laptop computer, a smart watch, a tablet, a standalone receiver, or any other computing device capable of executing application 106. In some embodiments, continuous analyte monitoring system 104 and/or analyte sensor application 106 transmit the analyte measurements to one or more other individuals having an interest in the health of the user (e.g., a family member or physician for real-time treatment and care of the user). An example continuous analyte monitoring system 104 is described in more detail with respect to FIG. 2.

Application 106 is a mobile health application (or similar software program) that is configured to receive and analyze analyte measurements from continuous analyte monitoring system 104. In particular, application 106 stores information about a user, including the user's analyte measurements, in a user profile 118 associated with the user for processing and analysis, as well as for use by therapy management engine 114 to provide therapy management support recommendations or guidance to the user.

Therapy management engine 114 refers to a set of software instructions with one or more software modules, including data analysis module (DAM) 116. In certain embodiments, therapy management engine 114 executes entirely on one or more computing devices in a private or a public cloud. In such embodiments, application 106 communicates with therapy management engine 114 over a network (e.g., Internet). In some other embodiments, therapy management engine 114 executes partially on one or more local devices, such as display device 107 and/or analyte sensor system 104, and partially on one or more computing devices in a private or a public cloud. In some other embodiments, therapy management engine 114 executes entirely on one or more local devices, such as display device 107 and/or analyte sensor system 104. As discussed in more detail herein, therapy management engine 114 can provide therapy management support recommendations to the user via application 106 for medical intervention, medications, and/or lifestyle changes, which may be altogether referred to as “treatment,” to improve the user's liver disease stage, prevent worsening liver disease, prevent the user from developing health complications related to liver disease, and/or treat health complications related to liver disease. Therapy management engine 114 provides therapy management support recommendations for medical intervention, medications, and/or lifestyle changes based on information included in user profile 118.

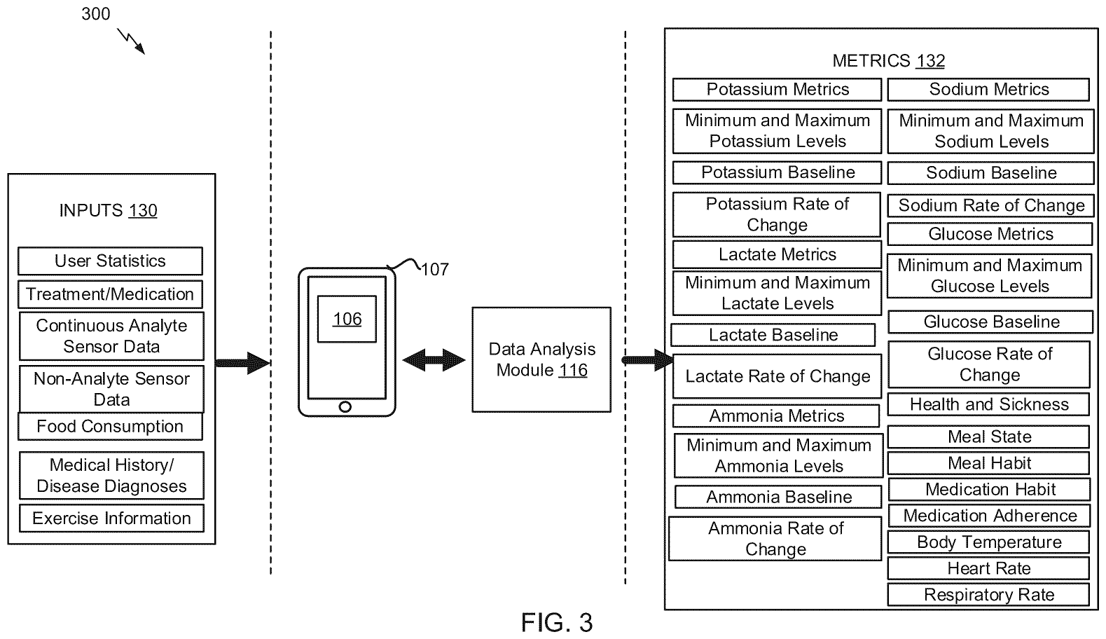

User profile 118 can include information collected about the user from application 106. For example, application 106 provides a set of inputs 130, including the analyte measurements received from continuous analyte monitoring system 104, that are stored in user profile 118. In certain embodiments, inputs 130 provided by application 106 include other data in addition to analyte measurements received from continuous analyte monitoring system 104. For example, application 106 can obtain additional inputs 130 through manual user input, one or more other non-analyte sensors or devices, non-continuous analyte lab test results (e.g., liver biopsy, metabolic assay panels (e.g., testing for elevated liver enzymes such as alkaline phosphatase (ALP), alanine transaminase (ALT), aspartate transaminase (AST), gamma-glutamyl transferase (GGT), etc.), Fibroscan results, electrolyte panels, urine pH tests, and blood tests for bilirubin, albumin and/or total protein, prothrombin time (PT), international normalized ration (INR), platelet count etc.), other applications executing on display device 107, etc. Non-analyte sensors and devices include one or more of, but are not limited to, an insulin pump, stretch sensor, body sound sensor, impedance sensor, an electrocardiogram (ECG) sensor, a heart rate monitor, a blood pressure sensor, a respiratory sensor, a thermometer, a sweat sensor, an ultrasound sensor, sensors or devices provided by display device 107 (e.g., accelerometer, camera, global positioning system (GPS), heart rate monitor, etc.), or other user accessories (e.g., a smart watch or fitness tracker), or any other sensors or devices that provide relevant information about the user. Inputs 130 of user profile 118 provided by application 106 are described in further detail below with respect to FIG. 3.

DAM 116 of therapy management engine 114 is configured to process the set of inputs 130 to calculate one or more metrics 132. Metrics 132, discussed in more detail below with respect to FIG. 3, can, at least in some cases, be generally indicative of the disease state of a user, such as one or more of the user's general analyte trends, trends associated with the health of the user, etc. In certain embodiments, metrics 132 are then used by therapy management engine 114 as input for providing liver disease therapy management for the user. In some examples, metrics 132 are compared against one or more predefined thresholds to determine a therapy. As shown, metrics 132 are also stored in user profile 118.

User profile 118 also includes demographic info 120, physiological info 122, disease progression info 124, and/or medication info 126. In certain embodiments, such information is provided through user input, obtained from one or more analyte sensors 202 or non-analyte sensors 206, or obtained from certain data stores (e.g., electronic medical records (EMRs), etc.). In certain embodiments, demographic info 120 includes one or more of the user's age, ethnicity, gender, etc. In certain embodiments, physiological info 122 includes one or more of the user's height, weight, and/or body mass index (BMI). In certain embodiments, disease progression info 124 includes information about a disease of a user, such as whether the user has been previously diagnosed with liver disease, a health complication related to liver disease, and/or have had symptoms of liver disease, such as a history of diabetes, kidney disease, sarcopenia, recurring infections, hyperglycemia, hypoglycemia, etc. In certain embodiments, information about a user's disease also includes the length of time since diagnosis, the level of disease control, level of compliance with disease management therapy, predicted liver function, other types of diagnosis (e.g., heart disease, hypertension, obesity), or measures of health (e.g., heart rate, exercise, sleep, etc.), and/or the like. In certain embodiments, user profile 118 includes information about the user's drug and/or alcohol consumption, which can affect a user's lactate metabolism and/or liver function.

In certain embodiments, medication info 126 includes information about the amount, frequency, and type of a medication taken by a user. In certain embodiments, the amount, frequency, and type of a medication taken by a user is time-stamped and correlated with the user's analyte levels, thereby, indicating the impact the amount, frequency, and type of the medication had on the user's analyte levels.

In certain embodiments, medication information includes information about the consumption of one or more drugs known to cause disease progression. These medications include those that damage the liver (e.g., affect lactic clearance) and/or lead to liver toxicity. One or more drugs known to damage the liver and/or lead to liver toxicity can include antibiotics such as amoxicillin/clavulanate, clindamycin, erythromycin, nitrofurantoin, rifampin, sulfonamides, tetracyclines, trimethoprim/sulfamethoxazole, and drugs used to treat tuberculosis (isoniazid and pyrazinamide), anticonvulsants such as tarbamazepine, thenobarbital, phenytoin, and valproate, antidepressants such as bupropion, fluoxetine, mirtazapine, paroxetine, sertraline, trazodone, and tricyclic antidepressants such as amitriptyline, antifungal drugs such as ketoconazole and terbinafine, antihypertensive drugs (e.g., drugs used to treat high blood pressure or sometimes kidney or heart disorder) such as captopril, enalapril, irbesartan, lisinopril, losartan, and verapamil, antipsychotic drugs such as phenothiazines (e.g., such as chlorpromazine) and risperidone, heart drugs such as amiodarone and clopidogrel, hormone regulation drugs such as anabolic steroids, birth control pills (oral contraceptives), and estrogens, pain relievers such as acetaminophen and nonsteroidal anti-inflammatory drugs (NSAIDs), and other drugs such as acarbose (e.g., used to treat diabetes), allopurinol (e.g., used to treat gout), antiretroviral therapy (ART) drugs (e.g., used to treat human immunodeficiency virus (HIV) infection), baclofen (e.g., a muscle relaxant), cyproheptadine (e.g., an antihistamine), azathioprine (e.g., used to prevent rejection of an organ transplant), methotrexate (e.g., used to treat cancer), omeprazole (e.g., used to treat gastroesophageal reflux), PD-1/PD-L1 inhibitors (e.g., anticancer drugs), statins (e.g., used to treat high cholesterol levels), nano-particle drug formulations, Adeno-associated viral gene therapies, and many types of chemotherapy, including immune checkpoint inhibitors.

In certain embodiments, medication information includes information about consumption of one or more drugs known to improve the body's metabolic conditions (e.g., improved organ function). Such drugs include ademetionine, avatrombopag, dehydroemetine, entecavir, glecaprevir and pibrentasvir, glucagon-like peptide 1 agonists (GLP-1), lamivudine, metadoxine, methionine, sodium-glucose cotransporter-2 (SGLT2), sofosbuvir, velpatasvir, and voxilaprevir, telbivudine, tenofovir, trientine, ursodeoxycholic acid, and the like.

The information regarding medication consumption can be input by a user, determined using electronic patient records, or can be detected using technologies that allow for detection of presence of certain chemicals in the body.

In certain embodiments, user profile 118 is dynamic because at least part of the information that is stored in user profile 118 is revised over time and/or new information is added to user profile 118 by therapy management engine 114 and/or application 106. Accordingly, information in user profile 118 stored in user database 110 provides an up-to-date repository of information related to a user.

User database 110, in some embodiments, refers to a storage server that operates in a public or private cloud. User database 110 can be implemented as any type of datastore, such as relational databases, non-relational databases, key-value datastores, file systems including hierarchical file systems, and the like. In some exemplary implementations, user database 110 is distributed. For example, user database 110 can comprise a plurality of persistent storage devices, which are distributed. Furthermore, user database 110 can be replicated so that the storage devices are geographically dispersed.

User database 110 includes user profiles 118 associated with a plurality of users who similarly interact with application 106 executing on the display devices 107 of the other users. User profiles stored in user database 110 are accessible to not only application 106, but therapy management engine 114, as well. User profiles in user database 110 can be accessible to application 106 and therapy management engine 114 over one or more networks (not shown). As described above, therapy management engine 114, and more specifically DAM 116 of therapy management engine 114, can fetch inputs 130 from user database 110 and compute a plurality of metrics 132 which can then be stored as application data 128 in user profile 118.

In certain embodiments, user profiles 118 stored in user database 110 are also stored in historical records database 112. User profiles 118 stored in historical records database 112 can provide a repository of up-to-date information and historical information for each user of application 106. Thus, historical records database 112 essentially provides all data related to each user of application 106, where data is stored according to an associated timestamp. The timestamp associated with information stored in historical records database 112 can identify, for example, when information related to a user has been obtained and/or updated.

Further, historical records database 112 can maintain time series data collected for users over a period of time, including for users who use continuous analyte monitoring system 104 and application 106. For example, analyte data for a user who has used continuous analyte monitoring system 104 and application 106 for a period of time to manage the user's liver disease and/or one or more health complications related to liver disease can have time series analyte data associated with the user maintained over the period of time. In certain embodiments, the period of time is 3 days, or 1 week, or one month, or one year, or five years, for example.

Further, in certain embodiments, historical records database 112 includes data for one or more users who are not users of continuous analyte monitoring system 104 and/or application 106. For example, historical records database 112 can include information (e.g., user profile(s)) related to one or more users analyzed by, for example, a healthcare physician (or other known method), and not previously diagnosed with liver disease and/or one or more health complications related to liver disease, as well as information (e.g., user profile(s)) related to one or more users who were analyzed by, for example, a healthcare physician (or other known method) and were previously diagnosed with (varying types and stages of) liver disease and/or one or more health complications related to liver disease. Data stored in historical records database 112 can be referred to herein as population data, which could include hundreds or thousands of data points for each one of thousands or millions of users in the user population. In other words, data stored in historical records database 112 and used in certain embodiments described herein could include gigabytes, terabytes, petabytes, exabytes, etc. of data.

Data related to each user stored in historical records database 112 can provide time series data collected over the disease lifetime of the user. For example, the data can include information about the user prior to being diagnosed with liver disease and/or one or more health complications related to liver disease and information associated with the user during the lifetime of the disease, including information related to each stage of the liver disease and/or one or more health complications related to liver disease as it progressed and/or regressed in the user, as well as information related to other diseases, such as hyperglycemia, hypoglycemia, kidney conditions and diseases, diabetes, recurring infections, hypertension, cardiovascular disease, or similar diseases that are co-morbid in relation to liver disease. Such information can indicate symptoms of the user, physiological states of the user, analyte levels of the user, states/conditions of one or more organs of the user, habits of the user (e.g., activity levels, food consumption, etc.), medication prescribed, etc. throughout the lifetime of the disease.

Although depicted as separate databases for conceptual clarity, in some embodiments, user database 110 and historical records database 112 operates as a single database. That is, historical and current data related to users of continuous analyte monitoring system 104 and application 106, as well as historical data related to users that were not previously users of continuous analyte monitoring system 104 and application 106, are stored in a single database. The single database can be a storage server that operates in a public or private cloud.

As mentioned previously, therapy management system 100 is configured to provide liver disease therapy management for a user using continuous analyte monitoring system 104. In certain embodiments, therapy management engine 114 is configured to provide real-time and or non-real-time liver disease therapy management guidance to the user (e.g., patient or host) and or others, including but not limited, to healthcare providers, family members of the user, caregivers of the user, researchers, artificial intelligence (AI) engines, and/or other individuals, systems, and/or groups supporting care or learning from the data.

For example, therapy management engine 114 can be used to collect information associated with a user in user profile 118 stored in user database 110, to perform analytics thereon for providing liver disease therapy management support, in some cases, providing recommendations to the user based on the therapy management support. Therapy management engine 114 can also be used to collect information associated with a user in user profile 118 to perform analytics thereon for providing liver disease therapy management support and providing one or more recommendations for medical intervention, medications, and/or lifestyle changes based, at least in part, on the therapy management support. User profile 118 can be accessible to therapy management engine 114 over one or more networks (not shown) for performing such analytics.

In certain embodiments, therapy management engine 114 further collects information from the user regarding recent habits in order to determine the accuracy of the liver disease therapy management support. For example, therapy management engine 114 can determine, from continuous analyte monitoring system 104, an abnormal pattern of analyte data consistent with worsening disease state (which may be due to various factors). In some examples, therapy management engine 114 determines, from continuous analyte monitoring system 104, an abnormal pattern of analyte data consistent with reduced liver function. During time periods of abnormal analyte patterns, therapy management engine 114 can review information collected from the user to determine whether the abnormal analyte pattern is a result of worsening disease state, worsening liver function, or a health complication or other factor related to or that may lead to a change in liver disease state (herein referred to as, “disease state”), or if the abnormal pattern may be a result of food or alcohol consumption, a new medication, or an illness or infection. Based on information from the patient, therapy management engine 114 can accurately determine whether abnormal analyte data is a result of worsening liver function, disease state, or a result of other factors such as temporary illness, alcohol consumption, or new medications.

In certain embodiments, therapy management engine 114 utilizes one or more trained machine learning models capable of providing liver disease therapy management support based on information that therapy management engine 114 has collected/received from user profile 118. In the illustrated embodiment of FIG. 1, therapy management engine 114 utilizes trained machine learning model(s) provided by a training system 140. Although depicted as a separate server for conceptual clarity, in certain embodiments, training system 140 and therapy management engine 114 operate as a single server or system. That is, the model can be trained and used by a single server and/or system, or can be trained by one or more servers and/or systems and deployed for use on one or more other servers and/or systems. In certain embodiments, the model is trained on one or many virtual machines (VMs) running, at least partially, on one or many physical services in relational and or non-relational database formats. In some examples, the machine learning model is trained and/or run on a local device such as the patient's device or a device of a caregiver, health care professional, or any other local device.

Training system 140 is configured to train the machine learning model(s) using training data, which can include data (e.g., from user profiles) associated one or more users (e.g., users or non-users of continuous analyte monitoring system 104 and/or application 106) previously diagnosed with varying stages of liver disease, previously diagnosed with one or more health complications related to liver disease, as well as users not previously diagnosed with liver disease and/or one or more health complications (e.g., healthy users, etc.). The training data can be stored in historical records database 112 and can be accessible to training system 140 over one or more networks (not shown) for training the machine learning model(s).

The training data refers to a dataset that has been featurized and labeled. For example, the dataset can include a plurality of data records, each including information corresponding to a different user profile stored in user database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model.

As an illustrative example, each relevant characteristic of a user, which is reflected in a corresponding data record, can be a feature used in training the machine learning model. Such features can include demographic information (e.g., age, gender, ethnicity, etc.), analyte information (e.g., sodium metrics, lactate metrics, ammonia metrics, potassium metrics, glucose metrics, etc.), non-analyte sensor information (e.g., stretch sensor data, impedance sensor data, body sound sensor data, etc.), medical history and/or disease information (e.g., cirrhosis, compensated and/or decompensated liver status, diabetes, kidney disease, sarcopenia, hypertension, hypotension, recurring infections, historical user liver metabolic panels, etc.), medication information, and/or any other information relevant to providing liver disease diagnosis and stage predictions, determining the presence of health complications related to liver disease, or to providing recommendations to users.

In addition, the data record is labeled with information the corresponding model is being trained to predict. In one example, if a model is being trained to predict whether the user's liver disease is worsening, then the data records in the training dataset are labeled with such diagnoses. In another example, if a model is being trained to output a prediction related to liver disease progression and/or a diagnosis of one or more health related complications associated with liver disease such as hepatic encephalopathy, sepsis, variceal bleeding, ascites, infection, etc., then the data records in the training dataset are labeled with one or more of such diagnoses. Note that, in one example, such a model is a multi-input single-output (MISO) model, configured to predict only whether the user's liver disease is worsening, in which case additional MISO models can be trained to each predict the liver disease progression, various health-related complications, including whether the condition is getting better or worsening, or the like. In another example, such a model is a multi-input multi-output (MIMO) model, configured to predict multiple disease-related predictions (e.g., liver disease progression, various health complication predictions, etc.).

The model(s) are then trained by training server and/or system 140 using the featurized and labeled training data. In particular, the features of each data record can be used as input into the machine learning model(s), and the generated output can be compared to label(s) associated with the corresponding data record. The model(s) can compute a loss based on the difference between the generated output and the provided label(s). This loss is then used to modify the internal parameters or weights of the model. By iteratively processing each data record corresponding to each historical user, the model(s) can be iteratively refined to generate accurate predictions of a liver disease progression, a diagnosis of health-related complications, or recommendations for medical intervention, medications, and/or lifestyle changes, etc.

As illustrated in FIG. 1, training system 140 deploys these trained model(s) to therapy management engine 114 for use during runtime. Training system 140 can include one or more computer systems, each including one or more servers or one or more other types of computing devices or systems. For example, therapy management engine 114 can obtain user profile 118 associated with a user and stored in user database 110, use information in user profile 118 as input into the trained model(s), and output a prediction indicative of the user's liver disease progression, presence of health complications related to liver disease and/or feedback related to liver disease and/or health complications (e.g., shown as output 144 in FIG. 1). Output 144 generated by therapy management engine 114 can indicate improvement or deterioration in the user's liver disease and/or health complications over time. Output 144 can be provided to the user (e.g., through application 106), to a caretaker of the user (e.g., a parent, a relative, a guardian, a teacher, a physical therapist, a fitness trainer, a nurse, etc.), to a physician or healthcare provider of the user, or any other individual that has an interest in the wellbeing of the user for purposes of improving the health of the user, such as, in some cases by effectuating recommended treatment and/or seeking medical intervention. Output 144 generated by therapy management engine 114 is stored in user database 110 and is utilized to train or re-train the trained model(s) and/or update a rules-based model.

In certain embodiments, output 144 generated by therapy management engine 114 is stored in user profile 118. Output 144 can be indicative of a user's current or future disease state, presence and/or severity of one or more health complications, and recommendations for medical intervention, medications, lifestyle changes, etc. As described herein, a current or future disease state can include a level of risk of liver disease, a progression or regression of liver disease, a presence of dysglycemia or organ dysfunctions that can be linked to liver disease, etc. Output 144 stored in user profile 118 can be continuously updated by therapy management engine 114 using the continuously provided continuous analyte and/or non-analyte data. Accordingly, for example, disease states and recommendations, originally stored as outputs 144 in user profile 118 in user database 110 and then passed to historical records database 112, can provide an indication of the progression or improvement of the disease state of a user over time, as well as provide an indication as to the effectiveness of different medical intervention, medications, and lifestyle changes recommended to the user to improve disease state.

For example, real-time continuous analyte data indicating the current analyte levels of a user can be measured by continuous analyte monitoring system 104 and used to generate a first set of values and/or metrics indicative of a user's liver health at a first time. The data can be stored in a local database or transmitted to a remote database to be stored in memory (e.g., in a first format). The metrics can also be provided in real time to a user (e.g., the user or health care provider) by generating a user interface or modifying a user interface at a user device. In a second time period, real-time continuous analyte data indicative of current analyte measurements relating to the second time period can be measured and used to generate similar values and/or metrics which can be stored in memory. One or more of the second time period metrics can also be used to generate data for display on a user interface (e.g., to a user or health care provider). The generated metrics are also, in some embodiments, stored locally in storage or transmitted remotely for storage in a database. The metrics from the first time period and second time period can be compared to derive indicators of user's liver health improvement that are not possible using point-in-time measurements.

In certain embodiments, a user's own historical data is used by training system 140 to train a personalized model for the user that provides therapy management support and insight around the user's medical history/current disease state, average analyte levels, etc. For example, in certain embodiments, a model trained based on population data is used to provide disease progression feedback to the user. However, after collecting personalized information (e.g., analyte sensor information, non-analyte sensor information, disease state, etc.) associated with the user, the personalized information can be used for further individualization or personalization of the model. For example, information obtained over time from the user can be used to more accurately determine liver disease progression, determine development of health complications related to liver disease, provide personalized recommendations for medical intervention, medications, and/or lifestyle changes, and monitor regression of disease state over time.

Further, a user's historical data can be used to generate a baseline to indicate progression or regression in the user's disease state and/or health complications based on various criteria. The criteria is selected from a list of available variables including the user's analyte metrics (e.g., baseline, rate of change, minimum and/or maximum levels), other information that indicate liver function, etc. As an illustrative example, a user's data, including a plurality of analyte measurements, over the course of 2 weeks during a previous time period (e.g., 1 day, 1 week, 1 month) can be used to generate a baseline that can be compared with the user's current data to identify whether the user's disease state and/or one or more health complications have changed (e.g., an improvement or worsening). In an additional or alternative embodiment, the model is further able to predict or project out the user's disease state and/or one or more health complications or their future improvement/deterioration based on the user's recent pattern of data (e.g., analyte data, non-analyte data, meal trends, exercise trends, etc.).

In certain embodiments, historical user population data based on users with decompensated liver disease and/or various health complications is used to generate a baseline to indicate progression or regression in the user's liver disease and/or health complications. As an illustrative example, historical user data, including a plurality of analyte measurements, over the course of 2 weeks during a previous time period (e.g., 1 day, 1 week, 1 month) can be used to generate a baseline (e.g., an average or mean calculation of continuous measurements) that can be compared with the user's current data (e.g., an average or mean generated from the continuous measurements generated using real time data indicative of the current analyte state of the user) to identify whether the user's liver disease and/or one or more health complications have improved. In certain other embodiments, known clinical evidence and/or observable data through clinical investigations of procedures is used to generate a baseline to indicate progression or regression in the user's liver disease and/or health complications. Similarly, the baseline can be generated from clinical evidence and/or observable data over the course of 2 weeks during a previous time period (e.g., 1 month ago), for example.

In certain embodiments, an AI/ML model is trained to provide a recommendation for medical intervention, medication, lifestyle, and other types of therapy management support recommendations to help the user improve their disease state and/or one or more health complications based on the user's historical data, including how different types of medication, food and/or activities impacted the user's liver function historically. In certain embodiments, an AI/ML model is trained to predict the underlying cause of certain improvements or deteriorations in the user's disease state and/or health complications. For example, application 106 can display a user interface with a graph that shows the user's analyte levels (e.g., disease state and/or presence of health complications) with trend lines and indicate, e.g., retrospectively, how the body's analyte levels affected the state of the patient's liver disease and/or corresponding health complications at certain points in time. Other depictions or trend indications, variables or metrics can also be provided for display to indicate the change.

In certain other embodiments, rules-based models are used. For example, a rules-based model can be used to map a user's inputs, analyte or non-analyte data from one or more continuous analyte sensor(s) 202 and/or non-analyte sensor(s) 206, and/or historical data to certain current or future liver disease and/or presence or progression of health complications, recommendations for medical intervention, medications, lifestyle changes, etc., using a rules library. In certain embodiments, a rules-based model maps certain inputs to disease state predictions, one or more health complication predictions, and/or recommendations for users with similar inputs historically. Some example rules are discussed herein in relation to block 402.

FIG. 2 is a diagram 200 conceptually illustrating an example continuous analyte monitoring system 104 including example continuous analyte sensor(s) with sensor electronics, in accordance with certain aspects of the present disclosure. For example, system 104 can be configured to continuously monitor one or more analytes of a user, in accordance with certain aspects of the present disclosure.

Continuous analyte monitoring system 104 in the illustrated embodiment includes sensor electronics module 204 and one or more continuous analyte sensor(s) 202 (individually referred to herein as continuous analyte sensor 202 and collectively referred to herein as continuous analyte sensors 202) associated with sensor electronics module 204. Sensor electronics module 204 is in wireless communication (e.g., directly or indirectly) with one or more of display devices 210, 220, 230, and 240. In certain embodiments, sensor electronics module 204 is also in wireless communication (e.g., directly or indirectly) with one or more medical devices, such as medical devices 208 (individually referred to herein as medical device 208 and collectively referred to herein as medical devices 208), and/or one or more other non-analyte sensors 206 (individually referred to herein as non-analyte sensor 206 and collectively referred to herein as non-analyte sensor 206).

In certain embodiments, a continuous analyte sensor 202 includes one or more sensors for detecting and/or measuring analyte(s). The continuous analyte sensor 202 can be a multi-analyte sensor configured to continuously measure two or more analytes or one or more single analyte sensors each configured to continuously measure a single analyte as a non-invasive device, a subcutaneous device, a transcutaneous device, a transdermal device, and/or an intravascular device. In certain embodiments, multiple single continuous analyte sensors in communication with the same sensor electronics module 204 and/or the same display device can be implemented in lieu of a single analyte sensor.

In certain embodiments, the continuous analyte sensor 202 is configured to continuously measure analyte levels of a user using one or more techniques, such as enzymatic techniques, chemical techniques, physical techniques, electrochemical techniques, spectrophotometric techniques, polarimetric techniques, calorimetric techniques, iontophoretic techniques, radiometric techniques, immunochemical techniques, and the like. The term “continuous,” as used herein, can mean fully continuous, semi-continuous, periodic, etc. In certain aspects, the continuous analyte sensor 202 provides a data stream indicative of the concentration of one or more analytes of the user. The data stream can include raw data signals, which are then converted into a calibrated and/or filtered data stream used to provide estimated analyte value(s) to the user.

In certain embodiments, the continuous analyte sensor 202 is a multi-analyte sensor, configured to continuously measure multiple analytes in a user's body. For example, in certain embodiments, the continuous multi-analyte sensor 202 is a single sensor configured to measure ammonia, lactate, glucose, sodium, and/or potassium in the user's body.

In certain embodiments, a single multi analyte sensor is used to measure multiple real time continuous stream of analyte measurements. In some examples, one or more multi-analyte sensors are used in combination with one or more single analyte sensors to provide the multiple real time continuous stream of analyte measurements. In certain other embodiments, one or more single analyte sensors are used, to provide the multiple real time continuous stream of analyte measurements. Information from each of the multi-analyte sensor(s) and/or single analyte sensor(s) can be combined to provide therapy management using methods described herein. In further embodiments, other non-contact and or periodic or semi-continuous, but temporally limited, measurements for physiological information are integrated into the system such as by including weight scale information or non-contact heart rate monitoring from a sensor pad under the user while in a chair or bed, through an infra-red camera detecting temperature and/or blood flow patterns of the user, and/or through a visual camera with machine vision for height, weight, or other parameter estimation without physical contact.

In certain embodiments, the continuous analyte sensor(s) 202 includes a percutaneous wire that has a proximal portion coupled to the sensor electronics module 204 and a distal portion with several electrodes, such as a measurement electrode and a reference electrode. The measurement (or working) electrode can be coated, covered, treated, embedded, etc., with one or more chemical molecules that react with a particular analyte, and the reference electrode can provide a reference electrical voltage. The measurement electrode can generate the analog electrical signal, which is conveyed along a conductor that extends from the measurement electrode to the proximal portion of the percutaneous wire that is coupled to the sensor electronics module 204. After the continuous analyte monitoring system 104 has been applied to epidermis of the patient, continuous analyte sensor(s) 202 penetrates the epidermis, and the distal portion extends into the dermis and/or subcutaneous tissue under epidermis. Other configurations of continuous analyte sensor(s) 202 can also be used, such as a multi-analyte sensor that includes multiple measurement electrodes, each generating an analog electrical signal that represents the concentration levels of a particular analyte.

Generally, a single-analyte sensor generates an analog electrical signal that is proportional to the concentration level of a particular analyte. Similarly, each multi-analyte sensor generates multiple analog electrical signals, and each analog electrical signal is proportional to the concentration level of a particular analyte. As an illustrative example, continuous analyte sensor 202 can include a single-analyte sensor configured to measure glucose concentration levels, and another single-analyte sensor configured to measure another analyte concentration level of the patient. As another illustrative example, continuous analyte sensor(s) 202 can include a single-analyte sensor configured to measure glucose concentration levels, and one or more multi-analyte sensors configured to measure lactate concentration levels, creatinine concentration levels, etc. As yet another illustrative example, continuous analyte sensor(s) 202 can include a multi-analyte sensor configured to measure glucose concentration levels, lactate concentration levels, creatinine concentration levels, etc.

Accordingly, continuous analyte sensor(s) 202 is configured to generate at least one analog electrical signal that is proportional to the concentration level of a particular analyte, and sensor electronics module 204 is configured to convert the analog electrical signal into an analyte sensor count values, calibrate the analyte sensor count values based on the sensitivity profile of the continuous analyte sensor(s) 202 to generate measured analyte concentration levels, and transmit the measured analyte concentration level data, including the measured analyte concentration levels, to a display device, such as display devices 210, 220, 230, and/or 240, via a wireless connection. For example, sensor electronics module 204 can be configured to sample the analog electrical signal at a particular sampling period (or rate), such as every 1 second (1 Hz), 5 seconds, 10 seconds, 30 seconds, 1 minute, 3 minutes, 5 minutes, etc., and to transmit the measured analyte concentration data to the display device at a particular transmission period (or rate), which can be the same as (or longer than) the sampling period, such as every 1 minute (0.016 Hz), 5 minutes, 10 minutes, 30 minutes, at the conclusion of the wear period, etc. Depending on the sampling and transmission periods, the measured analyte concentration data transmitted to the display device include at least one measured analyte concentration level having an associated time tag, sequence number, etc.

In certain embodiments, continuous analyte sensor(s) 202 incorporates a thermocouple to provide an analog temperature signal to the sensor electronics module 204. The temperature signal can be used to correct the analog electrical signal or the measured analyte data for temperature. The thermocouple is implemented in various alternative but equally compatible methods. It can be included in the sensing region, incorporated into the sensor electronics module 204, or contact the epidermis of the patient through openings in the adhesive pad.

The sensor electronics module 204 includes, inter alia, processor 233, storage element or memory 234, wireless transmitter/receiver (transceiver) 236, one or more antennas coupled to wireless transceiver 236, analog electrical signal processing circuitry, analog to-digital (A/D) signal processing circuitry, digital signal processing circuitry, a power source for continuous analyte sensor(s) 202 (such as a potentiostat), etc.

Processor 233 can be a general-purpose or application-specific microprocessor, an application-specific integrated circuit (ASIC), a field programmable gate array (FPGA), etc., that executes instructions to perform control, computation, input/output, etc, functions for the sensor electronics module 204. Processor 233 can include a single integrated circuit, such as a micro processing device, or multiple integrated circuit devices and/or circuit boards working in cooperation to accomplish the appropriate functionality. In certain embodiments, processor 233, memory 234, wireless transceiver 236, the A/D signal processing circuitry, and the digital signal processing circuitry are combined into a system-on-chip (SoC).

Generally, processor 233 can be configured to sample the analog electrical signal using the A/D signal processing circuitry at regular intervals (such as the sampling period) to generate analyte sensor count values based on the analog electrical signals produced by the continuous analyte sensor(s) 202, calibrate the analyte sensor count values based on the sensitivity profile of the continuous analyte sensor(s) 202 to generate measured analyte concentration levels, and generate measured analyte data from the measured analyte concentration levels, generate sensor data packages that include, inter alia, the measured analyte concentration level data. Processor 233 can store the measured analyte concentration level data in memory 234, and generate the sensor data packages at regular intervals (such as the transmission period) for transmission by wireless transceiver 236 to a display device, such as display devices 210, 220, 230, and/or 240. Processor 233 can also add additional data to the sensor data packages, such as supplemental sensor information that includes a sensor identifier, a sensor status, temperatures that correspond to the measured analyte data, etc. The sensor data packages are then wirelessly transmitted over a wireless connection to the display device. In certain embodiments, the wireless connection is a Bluetooth or Bluetooth Low Energy (BLE) connection. In such embodiments, the sensor data packages are transmitted in the form of Bluetooth or BLE data packets to the display device

Memory 234 includes volatile and nonvolatile medium. Memory 234 can include combinations of random access memory (RAM), dynamic RAM (DRAM), static RAM (SRAM), read only memory (ROM), flash memory, cache memory, and/or any other type of non-transitory computer-readable medium. Memory 234 stores one or more analyte sensor system applications, modules, instruction sets, etc. for execution by processor 233, such as instructions to generate measured analyte data from the analyte sensor count values, etc.

Additionally or alternatively, memory 234 stores certain sensor operating parameters 235, such as a calibration slope (or calibration sensitivity), a calibration baseline, etc. In particular, in certain implementations, information such as the calibration sensitivity, calibration baseline, and other information related to the sensitivity profile for the continuous analyte monitoring systems 104 are programmed into the sensor electronics module 204 during the manufacturing process, and then used to convert the analyte sensor electrical signals into measured analyte concentration levels. For example, as discussed above, the calibration slope is used to predict an initial in vivo sensitivity (M₀) and a final in vivo sensitivity (M_{f}), which are stored in memory 234 and used to convert the analyte sensor electrical signals into measured analyte concentration levels. In certain embodiments, calibration sensitivity (M_{CC}) 246 and/or calibration baseline 247 can be stored in memory 234.

In certain embodiments, sensor electronics module 204 can be physically connected to continuous analyte sensor(s) 202 and can be integral with (non-releasably attached to) to continuous analyte sensor(s) 202. Alternatively, sensor electronics module 204 can be physically connected to continuous analyte sensor(s) 202 and releasably attachable to continuous analyte sensor(s) 202. Sensor electronics module 204 can include hardware, firmware, and/or software that enable measurement of levels of analyte(s) via continuous analyte sensor(s) 202. For example, sensor electronics module 204 can include a potentiostat, an amperostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 210, 220, 230, and/or 240 are configured for displaying displayable sensor data, including analyte data, which can be transmitted by sensor electronics module 204. Display devices 210, 220, 230, or 240 can be implemented as a display such as a touchscreen display 212, 222, 232, and/or 242 for displaying sensor data to a user and/or for receiving inputs from the user. For example, a graphical user interface (GUI) can be presented to the user for such purposes. In certain embodiments, one or more of the display devices 210, 220, 230, and/or 240 include other types of user interfaces such as a voice user interface instead of, or in addition to, a touchscreen display for communicating sensor data to the user of the display device and/or for receiving user inputs. Display devices 210, 220, 230, and 240 are examples of display device 107 illustrated in FIG. 1 used to display sensor data to a user of the system of FIG. 1 and/or to receive input from the user.

In certain embodiments, one, some, or all of the display devices are configured to display or otherwise communicate (e.g., verbalize) the sensor data as it is communicated from the sensor electronics module (e.g., in a customized data package that is transmitted to display devices based on their respective preferences), without any additional prospective processing required for calibration and real-time display of the sensor data.

The plurality of display devices can include a custom display device specially designed for displaying certain types of displayable sensor data associated with analyte data received from sensor electronics module. In certain embodiments, the plurality of display devices are configured for providing alerts/alarms based on the displayable sensor data. Display device 210 is an example of such a custom device. In certain embodiments, one of the plurality of display devices is a smartphone, such as display device 220 which represents a mobile phone, using a commercially available operating system (OS), and configured to display a graphical representation of the continuous sensor data (e.g., including current and historic data). Other display devices can include other hand-held devices, such as display device 230 which represents a tablet, display device 240 which represents a smart watch or fitness tracker, medical device 208 (e.g., an insulin delivery device), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end user) for each particular display device. Accordingly, in certain embodiments, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor data.

As mentioned, sensor electronics module 204 can be in communication with a medical device 208. Medical device 208 can be a passive device in some example embodiments of the disclosure. Medical device 208 can be an insulin pump for administering insulin to a user. For a variety of reasons, it may be desirable for such an insulin pump to receive and track glucose values transmitted from continuous analyte monitoring systems 104, where continuous analyte sensor 202 is configured to measure at least glucose. In another example, medical device 208 can be a CPAP machine which may function as an indirect calorimeter. If the user uses a CPAP machine on a daily basis, utilizing a CPAP machine can be beneficial to gather indirect calorimetry readings in order to monitor for changes in liver metabolic function (e.g., as a function of overall metabolic function measured by indirect calorimetry).

Further, in some implementations of continuous analyte monitoring system 104, sensor electronics module 204 is also in communication with other non-analyte sensors 206. Non-analyte sensors 206 can include, but are not limited to, an altimeter sensor, an accelerometer sensor, a global positioning system (GPS) sensor, a temperature sensor, a respiration rate sensor, a stretch sensor, an impedance sensor, a body sound sensor, etc. Non-analyte sensors 206 can also include monitors such as heart rate monitors, blood pressure monitors, pulse oximeters, caloric intake monitors, indirect calorimetry devices, sweat sensors, photoplethysmography devices, and medicament delivery devices. One or more of these non-analyte sensors 206 can provide data to therapy management engine 114 described further below. In some aspects, a user can manually provide some of the data for processing by training system 140 and/or therapy management engine 114 of FIG. 1.

In certain embodiments, non-analyte sensors 206 further include sensors for measuring skin temperature, core temperature, sweat rate, and/or sweat composition.

In certain embodiments, the non-analyte sensors 206 are combined in another configuration, such as, for example, combined with one or more continuous analyte sensors 202. As an illustrative example, a non-analyte sensor, e.g., a stretch sensor, can be combined with a continuous ammonia and/or lactate sensor 202 to form an ammonia/lactate/stretch sensor used to transmit sensor data to the sensor electronics module 204 using common communication circuitry.

In certain embodiments, a wireless access point (WAP) is used to couple one or more of continuous analyte monitoring system 104, the plurality of display devices, medical device(s) 208, and/or non-analyte sensor(s) 206 to one another. For example, WAP 138 can provide Wi-Fi and/or cellular connectivity among these devices.

In certain embodiments, one or more of a variety of short-range communication protocols are used for wireless communication among devices depicted in diagram 200 of FIG. 2. For example, in some embodiments, the one or more of continuous analyte monitoring system 104, the plurality of display devices, medical device(s) 208, and/or non-analyte sensor(s) 206 communicate with one another using Near Field Communication (NFC) and/or Bluetooth/BLE protocols.

FIG. 3 illustrates example inputs and example metrics that are calculated based on the inputs for use by the therapy management system of FIG. 1, according to some embodiments disclosed herein. In particular, FIG. 3 provides a more detailed illustration of example inputs and example metrics introduced in FIG. 1.

FIG. 3 illustrates example inputs 130 on the left, application 106 and DAM 116 in the middle, and metrics 132 on the right. In certain embodiments, each one of metrics 132 corresponds to one or more values, e.g., discrete numerical values, ranges, or qualitative values (high/medium/low, stable/unstable, etc.). Application 106 obtains inputs 130 through one or more channels (e.g., manual user input, sensors, other applications executing on display device 107, an EMR system, etc.). As mentioned previously, in certain embodiments, inputs 130 are processed by DAM 116 to output a plurality of metrics, such as metrics 132. Inputs 130 and metrics 132 can be used by training system 140 and therapy management engine 114 to both train and deploy one or more machine learning models for providing liver disease therapy management for the user (e.g., host or patient), and other functionalities described herein.

In certain embodiments, starting with inputs 130, user statistics, such as one or more of age, gender, height, weight, BMI, body composition (e.g., % body fat), stature, build, or other information are also provided as an input. In certain embodiments, user statistics are provided through a user interface, by interfacing with an electronic source such as an electronic medical record, and/or from measurement devices. In certain embodiments, the measurement devices include one or more of a wireless, e.g., Bluetooth-enabled, weight scale and/or camera, which can, for example, communicate with the display device 107 to provide user data.

In certain embodiments, treatment/medication information is also provided as an input. Medication information can include information about the type, dosage, and/or timing of when one or more medications are to be taken by the user. Treatment information can include information regarding different lifestyle habits recommended by the user's physician. For example, the user's physician can recommend a user follow specific diet recommendations (e.g., types of calories consumed), exercise at a specific time during the day for a specific duration, eat a meal at certain days and/or times, or cut calories by 500 to 1,000 calories daily to improve analyte levels (e.g., lactate and/or glucose, for example) and therefore improve disease state. In certain embodiments, treatment/medication information is provided through manual user input.

In certain embodiments, analyte sensor data is also provided as input, for example, through continuous analyte monitoring system 104. In certain embodiments, analyte sensor data includes ammonia, lactate, sodium, calcium, potassium, and/or glucose levels measured by at least a single analyte sensor (or multi-analyte sensor) in continuous analyte monitoring system 104. In certain embodiments, analyte sensor data includes electrolyte and/or PH levels.

In certain embodiments, input is also received from one or more non-analyte sensors, such as non-analyte sensors 206 described with respect to FIG. 2. Input from such non-analyte sensors 206 includes information related to a heart rate, a respiration rate, oxygen saturation, blood pressure, or a body temperature (e.g. to detect illness, physical activity, etc.) of a user and/or measurements of variations, averages, derivatives, or any other multi-measurement analytical calculations between at least two points of non-analyte and/or analyte data. In certain embodiments, electromagnetic sensors also detect low-power radio frequency (RF) fields emitted from objects or tools touching or near the object, which can provide information about user activity or location.

In certain embodiments, food consumption information is also provided as input. Food consumption information can include information about one or more of meals, snacks, and/or beverages, such as one or more of the size, content (carbohydrate, fat, protein, etc.), sequence of consumption, and time of consumption. In certain embodiments, food consumption is provided by a user through manual entry, by providing a photograph through an application that is configured to recognize food types and quantities, and/or by scanning a bar code or menu. In various examples, meal size is manually entered as one or more of calories, quantity (“three cookies”), menu items (“Royale with Cheese”), and/or food exchanges (1 fruit, 1 dairy). In some examples, meal information is received via a convenient user interface provided by application 106.

In certain embodiments, food consumption information (the type of food (e.g., liquid or solid, snack or meal, etc.) and/or the composition of the food (e.g., carbohydrate, fat, protein, etc.)) is determined automatically based on information provided by one or more sensors. Some example sensors can include body sound sensors (e.g., abdominal sounds may be used to detect the types of meal, e.g., liquid/solid food, snack/meal, etc.), radio-frequency sensors, cameras, hyperspectral cameras, and/or analyte (e.g., glucose, lactate, etc.) sensors to determine the type and/or composition of the food.

In certain embodiments, medical history and/or disease diagnoses (e.g., liver disease, hepatic encephalopathy, sepsis, variceal bleeding, ascites, diabetes, kidney disease, hypertension, hypotension, sarcopenia, recurring infections, historical user liver metabolic panels, etc.) are provided as an input. For example, the user can have an existing diagnosis of liver disease and/or one or more health complications and this diagnosis can be provided through manual user input. In certain embodiments, disease diagnoses are also provided by interfacing with an electronic source such as an electronic medical record.

In certain embodiments, time is also provided as an input, such as time of day or time from a real-time clock. For example, in certain embodiments, input analyte data is timestamped to indicate a date and time when the analyte measurement was taken for the user.

User input of any of the above-mentioned inputs 130 can be provided through continuous analyte sensor system 104, non-analyte sensors 206, and/or a user interface, such a user interface of display device 107 of FIG. 1. As described above, in certain embodiments, DAM 116 determines or computes the user's metrics 132 based on inputs 130. An example list of metrics 132 is shown in FIG. 3.

In certain embodiments, potassium metrics are determined from sensor data (e.g., potassium measurements obtained from a continuous potassium sensor of continuous analyte monitoring system 104). For example, potassium metrics refer to time-stamped potassium measurements or values that are continuously generated and stored over time.

In certain embodiments, a minimum and maximum potassium level are determined from sensor data. For example, daily minimum and maximum potassium values for each day over a specified amount of time (e.g., a week or a month) are determined. In certain embodiments, the minimum and maximum potassium levels are determined based on an average minimum and maximum over a specified amount of time (e.g., a week or a month). In certain embodiments, DAM 116 continuously or periodically calculates a normal potassium range and time-stamp and store the corresponding information in the user's profile 118.

In other embodiments, a normal minimum and maximum potassium level are determined from population data (e.g., from data records or historical users with liver disease and/or health complications). In such embodiments, each user has personalized, customized, acceptable minimum and/or maximum potassium values, which are determined based on various time periods when the user is in a fasting state or during a meal, for example.

In certain embodiments, a potassium baseline is determined from sensor data (e.g., potassium measurements obtained from a continuous potassium sensor of continuous analyte monitoring system 104). A potassium baseline represents a user's normal potassium levels during periods where fluctuations in potassium production is typically not expected. A user's baseline potassium level is generally expected to remain constant over time, unless challenged through an action such as, for example, performing exercise (especially intense exercise), consuming food that is high in potassium, or administering a diuretic. Additionally, a user's baseline potassium level can also change based on the user's health, specifically an improvement or decline in liver health and/or kidney disease, for example. Further, each user can have a different potassium baseline. In certain embodiments, a user's potassium baseline is determined by calculating an average of potassium levels over a specified amount of time where fluctuations are not expected.

For example, the baseline potassium level for a user can be determined over a period of time when the user is sleeping, sitting in a chair, or other periods of time where the user is sedentary and not consuming food or medication which would reduce or increase potassium levels. In certain embodiments, DAM 116 continuously calculates a potassium baseline and time-stamp and store the corresponding information in the user's profile 118. In certain embodiments, DAM 116 calculates the potassium baseline using potassium levels measured over a period of time where the user is sedentary, the user is not consuming high potassium foods, and where no external conditions exist that would affect the potassium baseline. In certain embodiments, DAM 116 calculates the potassium baseline level by first determining a percentage of the number of potassium values measured during a specific time period that represent the lowest potassium values measured. DAM 116 can then take an average of this percentage to determine the potassium baseline level.

In certain embodiments, a potassium rate of change is determined from potassium levels (e.g., potassium measurements obtained from a continuous potassium sensor of continuous analyte monitoring system 104). A potassium rate of change refers to a rate that indicates how one or more time-stamped potassium measurements or values change in relation to one or more other time-stamped potassium measurements or values. Potassium rates of change can be determined over one or more seconds, minutes, hours, days, etc. Further, potassium rate of change can be positive, negative, or an absolute value. In certain embodiments, a potassium rate of change above or below a threshold is determined. Regular fluctuations above a threshold can indicate organ (e.g., liver) dysfunction.

In certain embodiments, lactate metrics are determined from sensor data (e.g., lactate measurements obtained from a continuous lactate sensor of continuous analyte monitoring system 104). For example, lactate metrics refer to time-stamped lactate measurements or values that are continuously generated and stored over time. In some examples, lactate metrics are also determined, for example, based upon historical data in particular situations, e.g., given a combination of food consumption and/or exercise.

In certain embodiments, a minimum and maximum lactate level are determined from sensor data. For example, daily minimum and maximum lactate values for each day over a specified amount of time (e.g., a week or a month) are determined. In certain embodiments, the minimum and maximum lactate levels are determined based on an average minimum and maximum over a specified amount of time (e.g., a week or a month). In certain embodiments, DAM 116 continuously or periodically calculates a normal lactate range and time-stamp and store the corresponding information in the user's profile 118.

In other embodiments, a normal minimum and maximum lactate level are determined from population data (e.g., from data records or historical users with liver disease and/or health complications). In such embodiments, each user has personalized, customized, acceptable minimum and/or maximum lactate values, which is determined based on various time periods when the user is in a fasting state or during a meal, for example.

In certain embodiments, a lactate baseline is determined from sensor data (e.g., lactate measurements obtained from a continuous lactate sensor of continuous analyte monitoring system 104). A lactate baseline represents a user's normal lactate levels during periods where fluctuations in lactate production is typically not expected. A user's baseline lactate level is generally expected to remain constant over time, unless challenged through an action such as consuming food that is high in lactate, for example. Additionally, a user's baseline lactate level can also change based on the user's health, specifically an improvement or decline in liver health, for example. Further, each user can have a different lactate baseline. In certain embodiments, a user's lactate baseline is determined by calculating an average of lactate levels over a specified amount of time where fluctuations are not expected.

For example, the baseline lactate level for a user can be determined over a period of time when the user is sleeping, sitting in a chair, or other periods of time where the user is sedentary and not consuming food or medication, or exercising, which would reduce or increase lactate levels. In certain embodiments, DAM 116 continuously, semi-continuously, or periodically calculates a lactate baseline and time-stamp and store the corresponding information in the user's profile 118. In certain embodiments, DAM 116 calculates the lactate baseline using lactate levels measured over a period of time where the user is sedentary (e.g., not exercising) and where no external conditions exist that would affect the lactate baseline. In certain embodiments, DAM 116 calculates the lactate baseline level by first determining a percentage of the number of lactate values measured during a specific time period that represent the lowest lactate values measured. DAM 116 can then take an average of this percentage to determine the lactate baseline level.

In certain embodiments, a lactate rate of change is determined from lactate levels (e.g., lactate measurements obtained from a continuous lactate sensor of continuous analyte monitoring system 104). A lactate rate of change refers to a rate that indicates how one or more time-stamped lactate measurements or values change in relation to one or more other time-stamped lactate measurements or values. Lactate rates of change can be determined over one or more seconds, minutes, hours, days, etc. Further, lactate rate of change can be positive, negative, or an absolute value. In certain embodiments, a lactate rate of change above or below a threshold is determined. A rapid rise in lactate over a threshold, not related to exercise or a meal, can be indicative of a health complication, such as infection.

In certain embodiments, ammonia metrics are determined from sensor data (e.g., ammonia measurements obtained from a continuous ammonia sensor of continuous analyte monitoring system 104). For example, ammonia metrics refer to time-stamped ammonia measurements or values that are continuously generated and stored over time. In some examples, ammonia metrics are also determined, for example, based upon historical data in particular situations, e.g., given a combination of food consumption, alcohol consumption, and/or exercise.

In certain embodiments, a minimum and maximum ammonia level are determined from sensor data. For example, daily minimum and maximum ammonia values for each day over a specified amount of time (e.g., a week or a month) are determined. In certain embodiments, the minimum and maximum ammonia levels are determined based on an average minimum and maximum over a specified amount of time (e.g., a week or a month). In certain embodiments, DAM 116 continuously or periodically calculates a normal ammonia range and time-stamp and store the corresponding information in the user's profile 118.

In other embodiments, a normal minimum and maximum ammonia level are determined from population data (e.g., from data records or historical users with liver disease and/or health complications). In such embodiments, each user has personalized, customized, acceptable minimum and/or maximum ammonia values, which are determined based on various time periods.

In certain embodiments, an ammonia baseline is determined from sensor data (e.g., ammonia measurements obtained from a continuous ammonia sensor of continuous analyte monitoring system 104). An ammonia baseline represents a user's normal ammonia levels during periods where fluctuations in ammonia production is typically not expected. A user's baseline ammonia level is generally expected to remain constant over time, unless challenged through an action such as, for example, performing exercise (especially intense exercise) or consuming food that is high in ammonia. Additionally, a user's baseline ammonia level can also change based on the user's health, such as with an improvement or decline in liver health and/or kidney disease, for example. Further, each user can have a different ammonia baseline. In certain embodiments, a user's ammonia baseline is determined by calculating an average of ammonia levels over a specified amount of time where fluctuations are not expected.

For example, the baseline ammonia level for a user is determined over a period of time when the user is sleeping, sitting in a chair, or other periods of time where the user is sedentary and not consuming food or medication which would reduce or increase ammonia levels. In certain embodiments, DAM 116 continuously, semi-continuously, or periodically calculates an ammonia baseline and time-stamp and store the corresponding information in the user's profile 118. In certain embodiments, DAM 116 calculates the potassium baseline using potassium levels measured over a period of time where the user is sedentary and where no external conditions exist that would affect the ammonia baseline. In certain embodiments, DAM 116 calculates the ammonia baseline level by first determining a percentage of the number of ammonia values measured during a specific time period that represent the lowest ammonia values measured. DAM 116 can then take an average of this percentage to determine the ammonia baseline level.

In certain embodiments, an ammonia rate of change is determined from ammonia levels (e.g., ammonia measurements obtained from a continuous ammonia sensor of continuous analyte monitoring system 104). An ammonia rate of change refers to a rate that indicates how one or more time-stamped ammonia measurements or values change in relation to one or more other time-stamped ammonia measurements or values. Ammonia rates of change can be determined over one or more seconds, minutes, hours, days, etc. Further, ammonia rate of change can be positive, negative, or an absolute value.

In certain embodiments, sodium metrics are determined from sensor data (e.g., sodium measurements obtained from a continuous sodium sensor of continuous analyte monitoring system 104). For example, sodium metrics refer to time-stamped sodium measurements or values that are continuously generated and stored over time. In some examples, sodium metrics are also determined, for example, based upon historical data in particular situations, e.g., given a combination of food consumption, insulin, and/or exercise.

In certain embodiments, a minimum and maximum sodium level are determined from sensor data. For example, a daily minimum and maximum sodium values for each day over a specified amount of time (e.g., a week or a month) are determined. In certain embodiments, the minimum and maximum sodium levels are determined based on an average minimum and maximum over a specified amount of time (e.g., a week or a month). In certain embodiments, DAM 116 continuously or periodically calculates a normal sodium range and time-stamp and store the corresponding information in the user's profile 118.

In other embodiments, a normal minimum and maximum sodium level are determined from population data (e.g., from data records or historical users with liver disease and/or health complications). In such embodiments, each user has personalized, customized, acceptable minimum and/or maximum sodium values, which is determined based on various time periods when the user is in a fasting state or during a meal, for example.

In certain embodiments, a sodium baseline is determined from sensor data (e.g., sodium measurements obtained from a continuous sodium sensor of continuous analyte monitoring system 104). A sodium baseline represents a user's normal sodium levels during periods where fluctuations in sodium production is typically not expected. A user's baseline sodium level is generally expected to remain constant over time, unless challenged through an action such as consuming food that is high in sodium, for example. Additionally, a user's baseline sodium level can also change based on the user's health, specifically an improvement or decline in liver health and/or kidney disease, for example. Further, each user can have a different sodium baseline. In certain embodiments, a user's sodium baseline is determined by calculating an average of sodium levels over a specified amount of time where fluctuations are not expected.

For example, the baseline sodium level for a user is determined over a period of time when the user is sleeping, sitting in a chair, or other periods of time where the user is sedentary and not consuming food or medication which would reduce or increase sodium levels. In certain embodiments, DAM 116 continuously, semi-continuously, or periodically calculates a sodium baseline and time-stamp and store the corresponding information in the user's profile 118. In certain embodiments, DAM 116 calculates the sodium baseline using sodium levels measured over a period of time where the user is sedentary, the user is not consuming high sodium foods, and where no external conditions exist that would affect the sodium baseline. In certain embodiments, DAM 116 calculates the sodium baseline level by first determining a percentage of the number of sodium values measured during a specific time period that represent the lowest sodium values measured. DAM 116 can then take an average of this percentage to determine the sodium baseline level.

In certain embodiments, a sodium rate of change is determined from sodium levels (e.g., sodium measurements obtained from a continuous sodium sensor of continuous analyte monitoring system 104). A potassium rate of change refers to a rate that indicates how one or more time-stamped sodium measurements or values change in relation to one or more other time-stamped sodium measurements or values. Sodium rates of change can be determined over one or more seconds, minutes, hours, days, etc. Further, sodium rate of change can be positive, negative, or an absolute value.

In certain embodiments, glucose metrics are determined from sensor data (e.g., glucose measurements obtained from a continuous glucose sensor of continuous analyte monitoring system 104). For example, glucose metrics refer to time-stamped glucose measurements or values that are continuously generated and stored over time. In some examples, glucose metrics are also determined, for example, based upon historical data in particular situations, e.g., given a combination of food consumption, insulin, and/or exercise.

In certain embodiments, a minimum and maximum glucose level are determined from sensor data. For example, daily minimum and maximum glucose values for each day over a specified amount of time (e.g., a week or a month) are determined. In certain embodiments, the minimum and maximum glucose levels are determined based on an average minimum and maximum over a specified amount of time (e.g., a week or a month). In certain embodiments, DAM 116 continuously or periodically calculates a normal glucose range and time-stamp and store the corresponding information in the user's profile 118.

In other embodiments, a normal minimum and maximum glucose level are determined from population data (e.g., from data records or historical users with liver disease). In such embodiments, each user has personalized, customized, acceptable glucose minimum and/or maximum glucose values, which are determined based on time periods when the user is in a fasting state or during a meal, for example.

In certain embodiments, a glucose baseline is determined from sensor data (e.g., glucose measurements obtained from a continuous glucose sensor of continuous analyte monitoring system 104). A glucose baseline represents a user's normal glucose levels during periods where fluctuations in glucose production is typically not expected. A user's baseline glucose level is generally expected to remain constant over time, unless challenged through an action such as consuming food or exercise by the user, for example. Additionally, a user's baseline glucose level can also change based on the user's health, specifically an improvement or decline in liver health. Further, each user can have a different glucose baseline. In certain embodiments, a user's glucose baseline is determined by calculating an average of glucose levels over a specified amount of time where fluctuations are not expected.

For example, the baseline glucose level for a user is determined over a period of time when the user is sleeping, sitting in a chair, or other periods of time where the user is sedentary and not consuming food or medication which would reduce or increase glucose levels. In certain embodiments, DAM 116 continuously, semi-continuously, or periodically calculates a glucose baseline and time-stamp and store the corresponding information in the user's profile 118. In certain embodiments, DAM 116 calculates the glucose baseline using glucose levels measured over a period of time where the user is sedentary, the user is not consuming glucose-heavy foods, and where no external conditions exist that would affect the glucose baseline. In certain other embodiments, DAM 116 uses glucose levels measured over a period of time where the user is, at least for a subset of the period of time, engaging in exercise and/or consuming glucose and/or an external condition exists that would affect the glucose baseline level. In this case, in some examples, DAM 116 first identifies which measured glucose values are to be used for calculating the baseline glucose level by identifying glucose values that may have been affected by an external event, such the consumption of food, exercise, medication, or other perturbation that would disrupt the capture of a glucose baseline measurement. DAM 116 can then exclude such measurements when calculating the glucose baseline level of the user. In some other examples, DAM 116 calculates the glucose baseline level by first determining a percentage of the number of glucose values measured during a specific time period that represent the lowest glucose values measured. DAM 116 then takes an average of this percentage to determine the glucose baseline level.

In certain embodiments, a glucose rate of change is determined from glucose levels (e.g., glucose measurements obtained from a continuous glucose sensor of continuous analyte monitoring system 104). A glucose rate of change refers to a rate that indicates how one or more time-stamped glucose measurements or values change in relation to one or more other time-stamped glucose measurements or values. Glucose rates of change can be determined over one or more seconds, minutes, hours, days, etc. Further, glucose rate of change can be positive, negative, or an absolute value. In certain embodiments, a glucose rate of change above or below a threshold is determined. A rapid rise in glucose outside of a threshold, not related to exercise or a meal, can be indicative of organ dysfunction (e.g., liver dysfunction).

In certain embodiments, a standard deviation of analyte levels (not shown) is determined from the analyte data. In some examples, a standard deviation of one or more analyte levels is determined based on the variability of one or more analyte levels as compared to an average analyte level over one or more time periods. In certain embodiments, a time-in-range metric (not shown) is determined from the analyte data. For example, with an established upper limit and lower limit, the time period during which the analyte data is between the upper and lower limits is determined. The time-in-range can be determined for individual instances of the analyte data being in range, or can be determined over a predetermined length of time (e.g., one day) for which each of the individual in range periods are summed.

In certain embodiments, analyte trends are determined based on analyte levels over certain periods of time. In certain embodiments, analyte trends (e.g., glucose, potassium, calcium, ammonia, or lactate trends) are determined based on analyte baselines over certain periods of time. In certain embodiments, analyte trends are determined based on absolute analyte level minimums over certain periods of time. In certain embodiments, analyte trends are determined based on absolute maximum analyte levels over certain periods of time. In certain embodiments, analyte trends are determined based on analyte level rates of change over certain periods of time. In certain embodiments, analyte trends are determined based on analyte baseline rates of change over certain periods of time. In certain embodiments, health and sickness metrics are determined, for example, based on one or more of user input (e.g., pregnancy information or known sickness and/or infection information), from physiologic sensors (e.g., temperature), activity sensors, or a combination thereof. In certain embodiments, based on the values of the health and sickness metrics, for example, a user's state is defined as being one or more of healthy, ill, rested, or exhausted.

In certain embodiments, the meal state metric indicates the state the user is in with respect to food consumption. For example, the meal state indicates whether the user is in one of a fasting state, pre-meal state, eating state, post-meal response state, or stable state. In certain embodiments, the meal state also indicates nourishment on board, e.g., meals, snacks, or beverages consumed, and is determined, for example from food consumption information, time of meal information, and/or digestive rate information, which can be correlated to food type, quantity, and/or sequence (e.g., which food/beverage was eaten first.).

In certain embodiments, meal habits metrics are based on the content and the timing of a user's meals. For example, if a meal habit metric is on a scale of 0 to 1, the better/healthier the meal consumed by the user, the higher the meal habit metric of the user will be to 1, in an example. Better/healthier meals can be defined as those that do not drive analyte (e.g., glucose) levels of a user out of a normal range for the user (e.g., 70-180 mg/dL glucose or the user's desired range). Also, the more the user's food consumption adheres to a certain time schedule, the closer their meal habit metric will be to 1, in the example. In certain embodiments, the meal habit metrics reflect the contents of a user's meals where, e.g., three numbers can indicate the percentages of carbohydrates, proteins and fats.

In certain embodiments, medication habit metrics are based on the user's prescribed medications and a determination of whether the prescribed medications have an effect on the user's analyte levels. For example, by analyzing a user's medication habits, DAM 116 can determine whether the user's medications impact the user's analyte measurements at a particular time. Based on the user's medication habits, DAM 116 can determine whether the user's analyte levels are a result of medication consumption or worsening liver function, for example. Medication habit metrics can be time-stamped so that they can be correlated with the user's analyte levels at the same time.

In certain embodiments, medication adherence is measured by one or more metrics that are indicative of how committed the user is towards their medication regimen. In certain embodiments, medication adherence metrics are calculated based on one or more of the timing of when the user takes medication (e.g., whether the user is on time or on schedule), the type of medication (e.g., is the user taking the right type of medication), and the dosage of the medication (e.g., is the user taking the right dosage).

In certain embodiments, body temperature metrics are calculated by DAM 116 based on inputs 130, and more specifically, non-analyte sensor data from a temperature sensor. In certain embodiments, heart rate metrics are calculated by DAM 116 based on inputs 130, and more specifically, non-analyte sensor data from a heart rate sensor. In certain embodiments, respiratory rate metrics are calculated by DAM 116 based on inputs 130, and more specifically, non-analyte sensor data from a respiratory rate sensor, sweat sensor, temperature sensor, or the like.

In certain embodiments, metrics 132 further include electrolyte metrics and/or pH metrics of the user.

Example Methods for Predicting a User's Liver Disease and/or Diagnosing and Staging Health Complications Related to Liver Disease Using Continuously Monitored Analyte Data

FIGS. 4-8 illustrate a flow diagrams of an example methods 400, 500, 600, 700, and 800, respectively, for providing (1) liver disease therapy management for a user (e.g., a patient or host wearing the analyte monitoring sensor(s)) and (2) recommendations to the user to improve the user's liver disease state and/or improve various health complications. Methods 400, 500, 600, 700, and 800 can be performed by therapy management system 100 to collect data, including for example, analyte data generated by a continuous analyte monitoring system 104, user information, and non-analyte sensor data mentioned above, to provide (1) liver disease therapy management and (2) recommendations for medical intervention, medications and/or lifestyle changes to improve the user's liver disease state and/or improve various health complications. Methods 400, 500, 600, 700, and 800 is described below with reference to FIGS. 1 and 2 and their components.

As discussed above, therapy management engine 114 can use one of a variety of models to provide (1) liver disease therapy management and/or (2) recommendations for medical intervention, medications, and/or lifestyle changes based on the inputs. As described above, the inputs can include analyte data (e.g., received by continuous analyte monitoring system 104), non-analyte data, and/or other user information (e.g., retrieved from the user's profile or received via user inputs). In embodiments where a rules-based model is used, the inputs to the model are mapped to certain liver disease therapy management, for example. For example, the rules-based model can take inputs and determine whether the user is suffering from worsening liver function, hepatic encephalopathy, ascites, variceal bleeding, and/or sepsis, for example.

As an example rule, a user can be determined to have worsening liver function based on inputs indicating that the user is diagnosed with liver disease and additional analyte data from continuous analyte monitoring system 104 demonstrating the user's lactate has continued to increase over time according to real time continuous analyte measurements. In another example, a user can be determined to be at risk of developing hepatic encephalopathy based on current and/or historical analyte data demonstrating decreasing potassium levels and increasing ammonia levels over time.

In certain embodiments, the rules become more granular, such that a combination of rules and/or inputs allow therapy management engine 114 to output a prediction of worsening liver function, for example. An example of such a rule can be to determine the user has decompensated liver disease based on the user's historical diagnosis of liver disease, a history of diabetes, and/or increasing ammonia levels over time to a threshold indicative of decompensated liver disease based on historical population data. Another example rule can be implemented by therapy management engine 114 to determine that the user is developing liver disease based on a prediction that the user is at risk of developing liver disease (e.g., based on a medical history of diabetes) and polysomnography device data demonstrating that the user's sleep patterns and/or circadian rhythms are worsening over time. Alternatively, if the user's sleep patterns and/or circadian rhythms improve over time, the user can be determined to not be developing liver disease.

In certain embodiments, instead of a rules-based model, an AI/ML model is used to output a prediction of worsening liver function and/or the development of one or more health complications related to liver disease, for example. Some or all of the inputs can be used as input into a model that is trained to output a disease prediction and/or disease stage for a user. In such cases, the model is trained using a dataset, including historical population-based data of many users, who have already been determined to have worsening liver disease, decompensated liver disease, and/or one or more health complications related to worsening liver disease. In such an example, the training dataset is labeled with such determinations.

Methods 400, 500, 600, 700, and 800 begin by therapy management engine 114 receiving analyte data from continuous analyte monitoring system 104. For example, continuous analyte monitoring system 104 can include a continuous potassium, lactate, ammonia, sodium, and/or glucose sensor 202 to measure the user's analyte levels, such as potassium, lactate, ammonia, sodium, and/or glucose levels. Further, therapy management engine 114 can receive data from user inputs. The user inputs can be received in a variety of ways. For example, the inputs can be received or retrieved from the user profile 118, which includes demographic info 120, physiological info 122, disease info 124, medication info 126, inputs 130, metrics 132, etc. Inputs can also be received as user input through the user interface of a display device 107.

Though each of FIGS. 4-8 primarily focus on a single analyte, the present disclosure also contemplates liver disease therapy management support and/or other types of recommendations discussed herein being provided by therapy management engine 114 based on two or more analytes. In other words, therapy management engine 114 can perform any combination of methods 400-800 (e.g., in parallel or a certain order) when providing therapy management support to the user.

Example Method for Monitoring Potassium Levels to Determine Worsening Liver Disease and/or Complications Related to Liver Disease

FIG. 4 illustrates a method 400 of providing liver disease therapy management support based on a user's potassium levels being monitored over time by continuous analyte monitoring system 104. Method 400 begins at block 404 with determining if the user is experiencing worsening liver disease and/or health complications related to worsening liver disease based on the user's potassium levels. In certain embodiments, monitoring the user's potassium levels includes optionally determining one or more potassium metrics, such as a potassium rate of change, potassium baseline, potassium minimum or maximum, etc. based on the measured potassium levels. At block 406, therapy management engine 114 determines whether the user's potassium levels are decreasing.

If the user is not experiencing decreasing potassium levels, therapy management engine 114 continues to block 420. At block 420, therapy management engine 114 determines whether the user's potassium levels are stable (e.g., not decreasing, or not increasing). If therapy management engine 114 determines the user's potassium levels are stable, therapy management engine 114 returns to block 404 to continue monitoring the user's potassium levels to determine changes in the user's potassium levels over time.

Alternatively, if therapy management engine 114 determines the user's potassium levels are not stable (e.g., they are decreasing, or increasing), therapy management engine 114 proceeds to block 422. At block 422, therapy management engine 114 determines whether the user's potassium levels are above the normal range (e.g., above 5.2 mmol/L, for example). If the user's potassium levels are above the normal range, therapy management engine 114 proceeds to block 424. At block 424, therapy management engine 114 provides feedback to the user to seek medical intervention for a risk of cardiac event (e.g., heart attack) or acute kidney failure. High potassium can indicate either a risk of cardiac event or kidney failure, however, additional testing and medical assessment can be required to determine the cause of the high potassium levels and/or prescribe appropriate treatment to the user.

Returning back to block 406, if therapy management engine 114 determines the user's potassium levels are decreasing therapy management engine 114 proceeds to block 408. At block 408, therapy management engine 114 determines whether the user's potassium levels are below a threshold (e.g., below 3 mmol/L, for example). If the user's potassium levels are not below a threshold, therapy management engine 114 returns to block 404 to continue monitoring the user's potassium levels. If the user's potassium levels are below a threshold, therapy management engine 114 proceeds to block 410. Alternatively or additionally, if the user's potassium levels are decreasing below a threshold (e.g., less than 3 mmol/L), the therapy management engine 114 provides feedback to the user to seek medical intervention for potential hypokalemia and/or cardiac complications. At block 410, therapy management engine 114 monitors ammonia and/or sodium levels to determine whether ammonia and sodium levels are normal, or ammonia and/or sodium levels are high or low. In certain embodiments, therapy management engine 114 receives ammonia and/or sodium levels from continuous analyte monitoring system 104, as described herein.

At block 412, if ammonia and sodium levels are normal (e.g., ammonia levels less than 45 μmol/L and sodium levels between 135-145 milliequivalents per liter (mEq/L), for example), therapy management engine 114 proceeds to block 416. At block 416, therapy management engine 114 continues monitoring potassium, sodium, and ammonia over time to identify changes in the user's analyte levels over time. Therapy management engine 114 returns to block 404 to continue monitoring the user's potassium levels.

At block 414, if ammonia levels are high and/or sodium levels are high or low (e.g., ammonia levels greater than 45 μmol/L and sodium levels above 145 mEq/L or below 135 mEq/L, for example) in addition to decreasing potassium levels, therapy management engine 114 proceeds to block 418. At block 418, therapy management engine 114 provide feedback to the user regarding the presence of, or risk of developing, hepatic encephalopathy. Therapy management engine 114 can recommend the user seek medical intervention for the treatment of hepatic encephalopathy.

In certain embodiments, at block 414, therapy management engine 114 also determines, based on ammonia rate of change, whether ammonia levels are rapidly increasing, which can be indicative of an acute health complication, or if ammonia levels are gradually changing over time, which can be indicative of a general decline in liver function. A rapid increase in ammonia levels can be indicative of an acute health complication, while ammonia levels gradually changing over time can indicate general liver function or decline over time.

Example Method for Monitoring Lactate Levels to Determine Worsening Liver Disease and/or Complications Related to Liver Disease

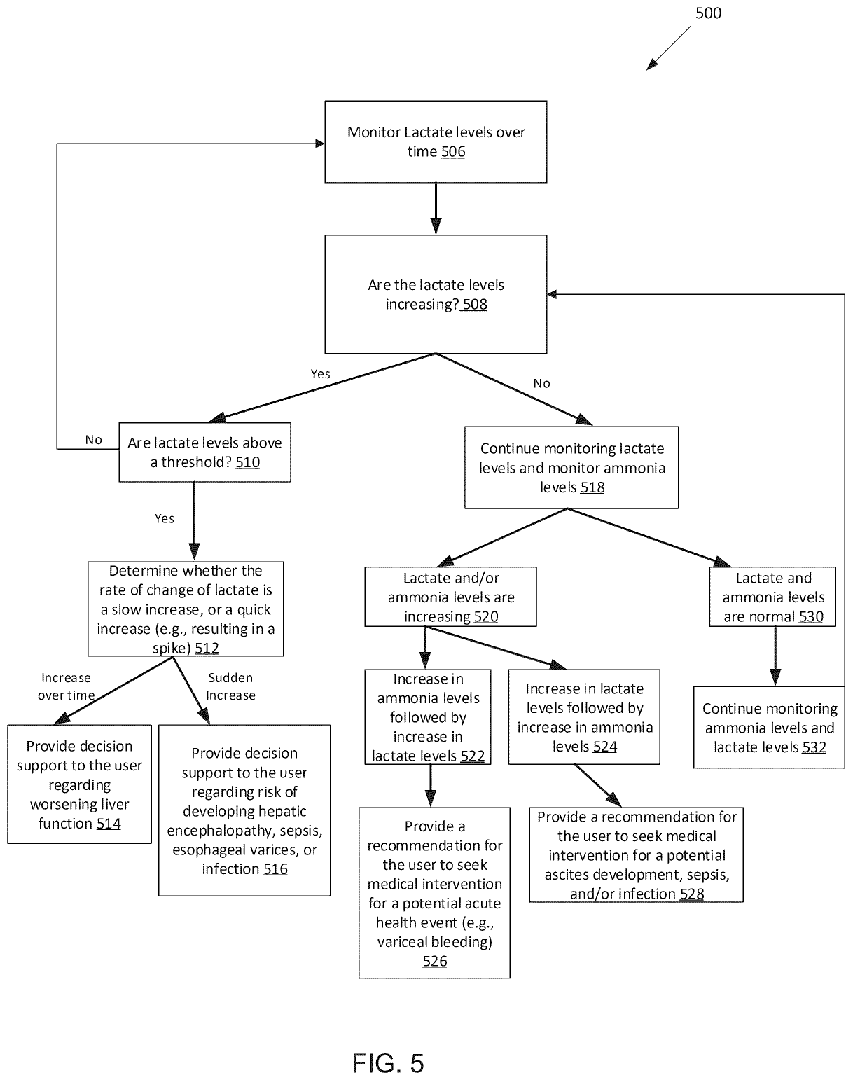

FIG. 5 illustrates a method 500 of providing liver disease therapy management support based on a user's lactate levels being monitored over time by continuous analyte monitoring system 104. Method 500 begins at block 506 with determining if the user is experiencing worsening liver disease and/or health complications related to worsening liver disease based on the user's lactate levels. In certain embodiments, monitoring the user's lactate levels includes optionally determining one or more lactate metrics, such as lactate rate of change, lactate baseline, lactate minimum or maximum, etc. based on the measured lactate levels. At block 508, therapy management engine 114 determines whether the user's lactate levels are increasing.

As therapy management engine 114 determines whether the user's lactate levels are increasing, therapy management engine 114 refers to non-analyte sensor data, other analyte sensor data, and/or user inputs to determine whether the increase in lactate is a result of a meal and/or an exercise session. For example, a non-analyte sensor (e.g., a fitness tracker and/or a body sounds sensor, for example) can assist therapy management engine 114 in in identifying when the user's lactate levels are increasing due to an exercise session or a meal. Based on the determination that the user's lactate levels are increasing due to an exercise session or a meal, therapy management engine 114 determines the increasing lactate levels during that time period are not related to worsening liver disease and/or health complications related to worsening liver disease. The therapy management engine 114 can further determine whether the user's lactate levels return to normal after a time period (e.g., 3 hours) following an exercise session or a meal. If the user's lactate levels do not return to normal, therapy management engine 114 provides feedback to the user to seek medical intervention for a potential health complication.

In certain embodiments, therapy management engine 114 provides feedback to the user, based on increasing lactate levels at rest, that the user should seek medical intervention for declining liver function and/or infection. In certain embodiments, the user is instructed to complete an oral lactate tolerance test (e.g., the user is instructed to consume a food or supplement high in lactate) in order to determine the lactate level rate of increase, lactate level absolute value, lactate level rate of decrease, etc. Lactate data resulting from the oral lactate tolerance test can function as a baseline to compare with future lactate metrics at rest or to compare with future oral lactate tolerance tests.

In certain other embodiments, if therapy management engine 114 determines the user is experiencing increasing lactate at rest (e.g., not due to a meal or an exercise session, for example), or an increase in lactate levels that persist over extended periods of time, therapy management engine 114 proceeds to block 510 to determine if the user's lactate levels are above a threshold (e.g., 2 mmol/L, for example). If the user's lactate levels are not above a threshold, therapy management engine 114 returns to block 506 to continue monitoring lactate levels over time. If the user's lactate levels are above a threshold, therapy management engine 114 proceeds to block 512. At block 512, therapy management engine 114 determines whether the rate of change of lactate levels is a slow increase or a quick increase (e.g., resulting in a spike of lactate concentration). In certain embodiments, a slow increase is a steady increase in lactate levels over several weeks and a quick increase is an increase in lactate over several hours, for example. If the user's lactate levels are increasing slowly over time, therapy management engine 114 proceeds to block 514. At block 514, therapy management engine 114 provides therapy management support to the user regarding worsening liver function based on the slow increase in lactate levels over time. Therapy management engine 114 can provide a recommendation to the user to seek medical intervention to improve liver function to prevent the progression of liver disease to decompensated liver disease.

Alternatively, at block 516, if the user's lactate levels suddenly increase (e.g., as demonstrated by the user's lactate rate of change over several hours), therapy management engine 114 provides therapy management support to the user regarding the presence of or risk of developing hepatic encephalopathy, sepsis, esophageal varices, other bleeding complications, or infection. With reference to block 516, “sudden” increases in lactate levels refers to increases in lactate levels between about 10% and about 25%, or between about 15 and about 20%, every few hours, as compared to lactate level changes caused by, for example, performing exercise or consuming a meal, which can result in severalfold changes in lactate level within a few minutes or hours. By providing therapy management support to the user regarding the presence of and/or risk of developing one or more health complications related to worsening liver disease, the user can seek medical intervention to determine the specific cause of the elevated lactate levels and a health care professional can provide appropriate recommendations to the user based on their determined disease state.

In certain embodiments, where therapy management engine 114 determines the user's lactate levels are increasing, therapy management engine 114 also determines the user's lactate clearance rate as the user clears lactate from the body following an increase in lactate levels. Therapy management engine 114 can monitor the user's lactate clearance rate over the subsequent days following the lactate increase. If the user's lactate clearance rate is slower than the user's historical lactate clearance rate and/or slower than the average lactate clearance rate of a healthy population, therapy management engine 114 provides a recommendation to the user to seek medical intervention for a potential decline in liver and/or kidney function.

Returning to block 508, therapy management engine 114 determines that the user's lactate levels are not increasing and proceed to block 518. At block 518, therapy management engine 114 continues monitoring lactate levels and begin monitoring ammonia levels. At block 530, if lactate and ammonia levels are normal (e.g., lactate levels below 2 mmol/L, and/or ammonia levels between 10-30 μmol/L, for example), therapy management engine 114 returns to block 506 to continue monitoring lactate levels to determine whether lactate levels are increasing, as discussed in reference to block 508.

Alternatively, if lactate and/or ammonia levels are increasing, at block 520, therapy management engine 114 proceeds to block 522 or block 524. Whether therapy management engine 114 proceeds to block 522 or block 524 depends on whether the user is experiencing an increase in lactate levels followed by an increase in ammonia levels, or an increase in ammonia levels followed by an increase in lactate levels. At block 522, if the user is experiencing an increase in ammonia levels followed by an increase in lactate levels, therapy management engine 114 proceeds to block 526 to provide liver disease therapy management support to the user. For example, rapid rises in ammonia levels of the user can indicate variceal bleeding or other gastrointestinal bleeding, depending on if an increase in lactate levels precedes or ensues the rise in ammonia levels. At block 526, therapy management engine 114 provides a recommendation to the user to seek medical intervention for a potential acute health event (e.g., variceal bleeding). At block 524, if the user is experiencing an increase in lactate levels followed by an increase in ammonia levels, therapy management engine 114 proceeds to block 528. At block 528, therapy management engine 114 provides a recommendation to the user to seek medical intervention for the presence of or risk of developing ascites, sepsis, and/or a bacterial positive infection.

In certain embodiments, therapy management engine 114 utilizes data from non-analyte sensors, such as non-analyte sensors 206, in addition to analyte data (e.g., lactate and/or ammonia levels) to assist therapy management engine 114 in determining when the user is suffering from ascites. For example, liver disease, congestive heart failure, nephrotic syndrome, cancer, pancreatitis, Budd-Chiari Syndrome, Meigs' Syndrome, tuberculosis, protein deficiency, and occasionally trauma can cause ascites, a buildup of fluid in spaces within the user's abdomen, which can be quantified or detected using a non-analyte sensor such as a stretch sensor, a body sound sensor, an impedance sensor, and/or an ultrasound sensor, such as a handheld ultrasound device. When data from a stretch sensor, a body sound sensor, and/or an impedance sensor is combined with lactate and/or ammonia levels, therapy management engine 114 can determine the type of health complication the user is suffering from when the user is experiencing an increase in lactate levels followed by an increase in ammonia levels.

Example Method for Monitoring Ammonia Levels to Determine Worsening Liver Disease and/or Complications Related to Liver Disease

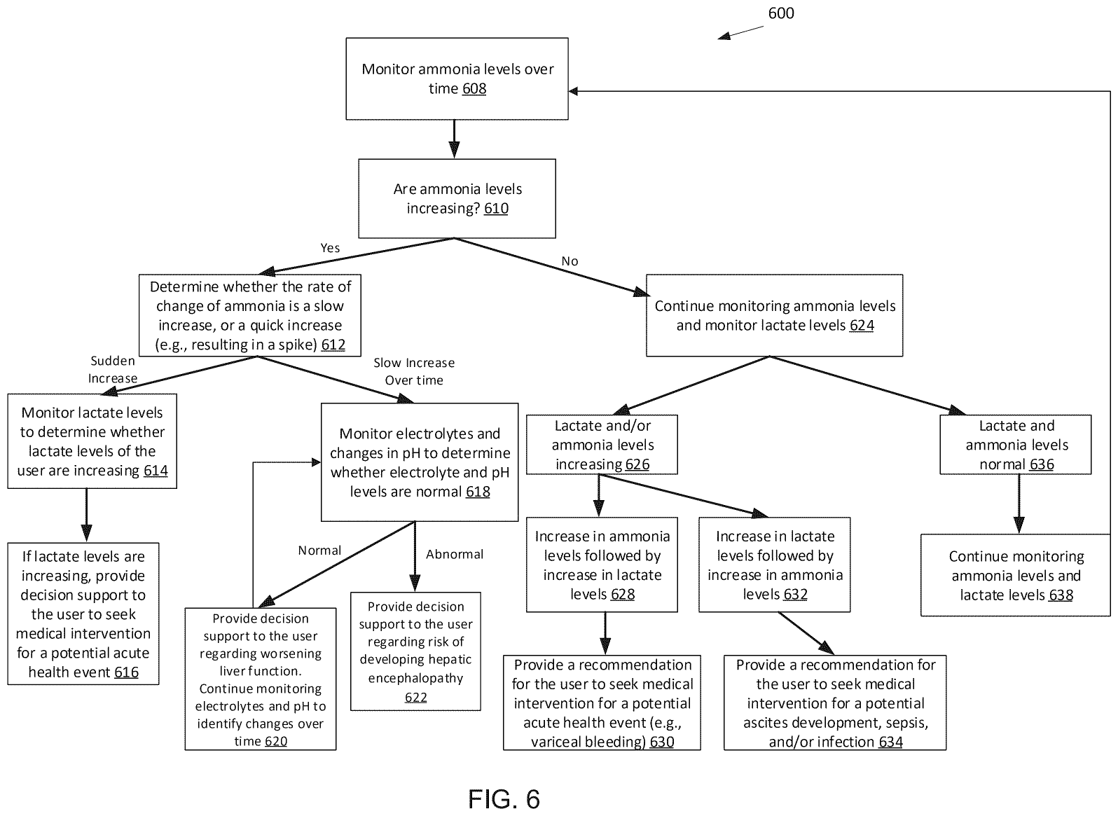

FIG. 6 illustrates a method 600 of providing liver disease therapy management support based on a user's ammonia levels being monitored over time by continuous analyte monitoring system 104. Method 600 begins at block 608 with determining if the user is experiencing worsening liver disease and/or health complications related to worsening liver disease based on the user's ammonia levels. In certain embodiments, monitoring the user's ammonia levels includes determining one or more ammonia metrics, such as ammonia rate of change, ammonia baseline, ammonia minimum or maximum, etc. based on the measured ammonia levels. At block 610, therapy management engine 114 determines whether the user's ammonia levels are increasing over time.

If the user's ammonia metrics demonstrate the user's ammonia levels are increasing, therapy management engine 114 continues to block 612. At block 612, therapy management engine 114 determines whether the rate of change of ammonia is a slow increase or a quick increase (e.g., resulting in a spike in ammonia concentration). At block 614, if the user's ammonia levels suddenly increase (e.g., as demonstrated by the user's ammonia rate of change), therapy management engine 114 begins to monitor lactate levels to determine whether the lactate levels of the user are increasing. Further, at block 614, if ammonia levels are increasing rapidly, therapy management engine 114 provides feedback to the user to seek medical intervention for a risk of developing hepatic encephalopathy, without further data regarding the user's lactate levels. At block 616, if the user's lactate levels are increasing, therapy management engine 114 provides a recommendation to the user to seek medical intervention for a potential acute health event (e.g., variceal bleeding).

Alternatively, if the user's ammonia levels are increasing slowly over time, therapy management engine 114 proceeds to block 618. At block 618, therapy management engine 114 monitors electrolyte levels and monitor for changes in pH to determine whether the user's electrolyte and pH levels are normal (e.g., within a normal range for the user). Changes in the user's electrolyte levels and/or PH balance in the body may compromise the user's blood brain barrier. A compromised blood brain barrier may allow toxins (e.g., ammonia) to enter the brain. Over time, a compromised blood brain barrier may put the user at risk of developing or cause the user to develop hepatic encephalopathy. In addition to monitoring pH and electrolyte levels to determine the integrity of the user's blood brain barrier, therapy management engine 114 monitors glucose to determine whether the user is experiencing periods of hyperglycemia and/or hypoglycemia, which can also contribute to a compromised blood brain barrier.

If therapy management engine 114 determines the user's electrolyte levels, PH levels, and glucose levels are normal (e.g., within a specified range based on historical data from a healthy user population, for example), therapy management engine 114 proceeds to block 620. At block 620, therapy management engine 114 provides therapy management support to the user regarding worsening liver function. Additionally, therapy management engine 114 returns to block 618 to continue monitoring the user's electrolyte and pH levels to determine when the user is experiencing changes in electrolyte levels, pH levels, and/or glucose levels over time.

In certain embodiments, in addition to continuing to monitor the user's electrolyte, pH, and glucose levels, therapy management engine 114 recommends the user seek medical intervention even when ammonia levels are increasing slowly over time, in the absence of abnormal electrolyte levels and/or PH levels. Even in cases of slow ammonia level increase, the user's liver may not be excreting ammonia properly, which can demonstrate worsening liver disease and/or potential development of a health complication over time.

Alternatively, if therapy management engine 114 determines at least one of the user's electrolyte, pH, and/or glucose levels are abnormal (e.g., outside of an expected range or not within the expected levels for the user), therapy management engine 114 proceeds to block 622. At block 622, therapy management engine 114 provides therapy management support to the user regarding the risk of developing hepatic encephalopathy.

However, returning to block 610, if therapy management engine 114 determines the user's ammonia levels are not increasing (e.g., ammonia levels are stable and/or decreasing), therapy management engine 114 continues to block 624. At block 624, therapy management engine 114 continues monitoring ammonia levels and begin monitoring lactate levels over time to identify changes in the user's ammonia and/or lactate levels. At block 636, if the user continues to experience normal ammonia and/or lactate levels, at block 638, therapy management engine 114 continues monitoring ammonia levels and lactate levels and return to block 608 as described above.

Alternatively, if the user experiences increasing lactate and/or ammonia levels over time, therapy management engine 114 proceeds to block 626. At block 626, therapy management engine 114 then determines whether ammonia levels are increasing followed by an increase in lactate levels, or whether lactate levels increase followed by an increase in ammonia levels, as discussed in reference to block 518 of FIG. 5. At block 628, if a user's ammonia levels increase followed by an increase in lactate levels, therapy management engine 114 proceeds to block 630. At block 630, therapy management engine 114 provides a recommendation to the user to seek medical intervention for a potential acute health event (e.g., variceal bleeding).

Alternatively, at block 632, if a user's lactate levels increase followed by an increase in ammonia levels, therapy management engine 114 proceeds to block 634. At block 634, therapy management engine 114 provides a recommendation to seek medical intervention for the presence and/or development of ascites, sepsis, and/or infection. If the user's lactate levels increase followed by an increase in ammonia levels, therapy management engine 114 further determines the user has a bacterial positive infection. However, if the user's lactate levels increase and ammonia levels do not increase over time, therapy management engine 114 can determine the user has sepsis and/or an infection, however, the infection can be determined to be a bacterial-negative infection.

As discussed herein, ammonia levels can be measured via continuous analyte monitoring system 104. Additionally or alternatively, ammonia levels can be monitored through the user's breath. In addition to ammonia measurements, the user's breath may also be utilized to monitor nitric oxide levels in the user's breath. Nitric oxide may be present in increasing concentrations in a user with ascites, a user with liver dysfunction, a user with sepsis, and/or a user with variceal bleeding (e.g., esophageal varices). As such, therapy management engine 114 can determine whether a user has or is developing sepsis, variceal bleeding, ascites, or worsening liver function based on analyte levels as described herein, in addition to nitric oxide levels in the user's breath sample and/or the change in levels of nitric oxide in the user's breath sample over time.

Example Method for Monitoring Sodium Levels to Determine Worsening Liver Disease and/or Complications Related to Liver Disease

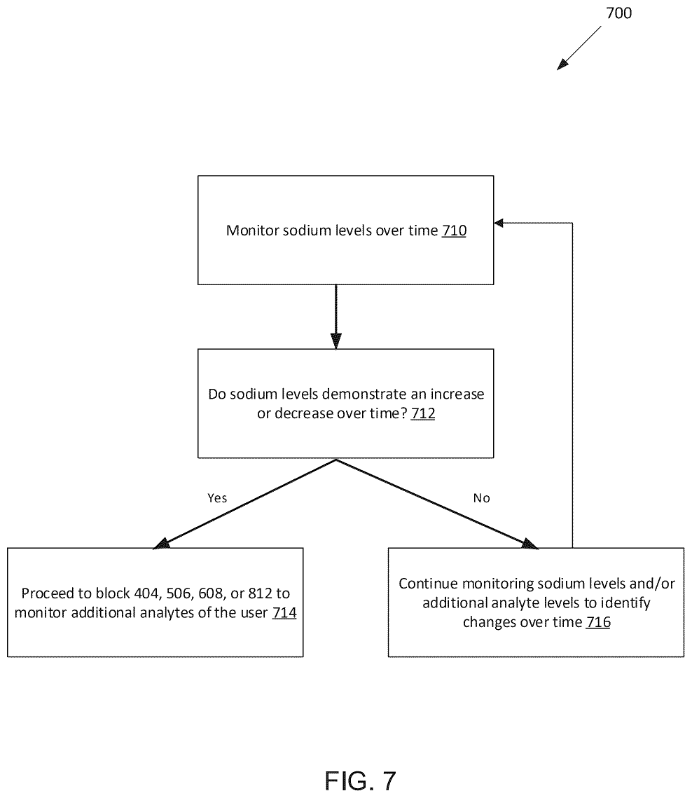

FIG. 7 illustrates a method 700 of providing liver disease therapy management support based on a user's sodium levels being monitored over time by continuous analyte monitoring system 104. Method 700 begins at block 710 with determining if the user is experiencing worsening liver disease and/or health complications related to worsening liver disease based on the user's sodium levels. In certain embodiments, monitoring the user's sodium levels includes determining one or more sodium metrics, such as sodium rate of change, sodium baseline, sodium minimum or maximum, etc. based on the measured sodium levels. At block 712, therapy management engine 114 determines whether the user's sodium levels are increasing or decreasing over time.

At block 714, if the user's sodium levels are increasing or decreasing, therapy management engine 114 proceeds to block 404, 506, 608, or 812 to monitor additional analytes of the user to identify analyte data and patterns consistent with worsening liver disease and/or health complications related to liver disease as described in reference to FIGS. 4, 5, 6, and 8.

Alternatively, at block 716, if the user's sodium levels are stable (e.g., sodium levels are not increasing or decreasing), therapy management engine 114 returns to block 710 to continue monitoring sodium levels of the user to identify changes in the user's sodium levels over time.

Example Method for Monitoring Glucose Levels to Determine Worsening Liver Disease and/or Complications Related to Liver Disease

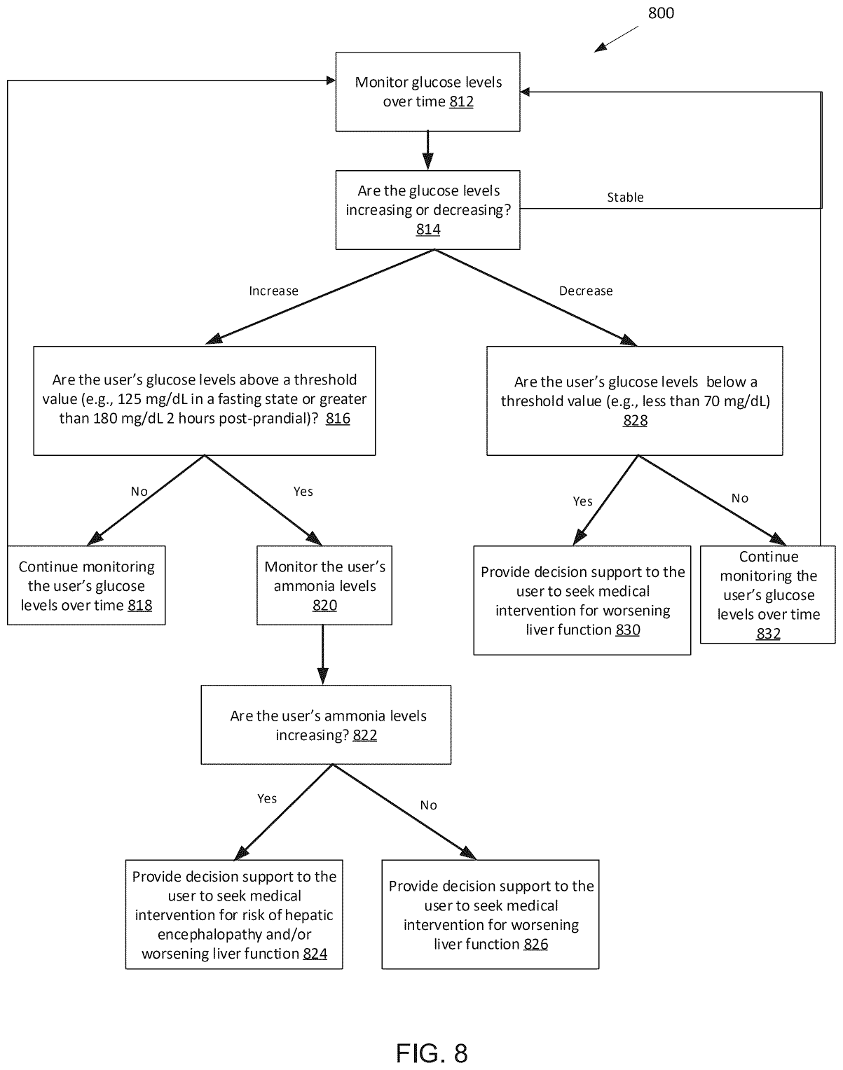

FIG. 8 illustrates a method of providing liver disease therapy management support based on a user's glucose levels being monitored over time by continuous analyte monitoring system 104. Method 800 begins at block 812 with determining if the user is experiencing worsening liver disease and/or health complications related to worsening liver disease based on the user's glucose levels. In certain embodiments, monitoring the user's glucose levels includes determining one or more glucose metrics, such as glucose rate of change, glucose baseline, glucose minimum or maximum, glucose standard deviation, glucose time in range, etc. based on the measured glucose levels. At block 814, therapy management engine 114 determines if the user's glucose levels are increasing, decreasing, or stable over time. In certain embodiments, therapy management engine 114 also determines whether the user's glucose time in range is decreasing. A decreasing glucose time in range can further support the determination that the is suffering from poor glucose control. In certain embodiments, if the user's glucose control decreases over time, therapy management engine 114 provides feedback to the user regarding worsening liver function.

If the user's glucose levels are increasing, at block 816, therapy management engine 114 determines if the user's glucose levels are above a threshold (e.g., greater than 125 mg/dL in a fasting state, or greater than 180 mg/dL after 2 hours following a meal, for example). In determining the appropriate threshold to compare with the user's glucose levels, therapy management engine 114 can utilize additional non-analyte sensor data and/or user inputs to determine whether the user is in a meal or fasting state. At block 818, if the user's glucose levels are not above a threshold value, therapy management engine 114 returns to block 812 and continue monitoring the user's glucose levels over time. In certain embodiments, therapy management engine 114 monitors the user's glucose levels over several days (e.g., 4 days) or months to monitor for changes that can indicate a decline in liver function and/or development of one or more health complications.

Alternatively, at block 820, if the user's glucose levels over time are above a threshold as described in reference to block 816, therapy management engine 114 monitors the user's ammonia levels at block 820. At block 822, therapy management engine 114 determines whether the user's ammonia levels are increasing in addition to increasing glucose levels. If the user's ammonia levels are increasing, therapy management engine 114 proceeds to block 824. At block 824, therapy management engine 114 provides therapy management support to the user to seek medical intervention for risk of developing hepatic encephalopathy and/or worsening liver function which may lead to worsening (e.g., decompensated) liver disease. At block 826, if the user's ammonia levels are not increasing in addition to increasing glucose levels, therapy management engine 114 provides therapy management support to the user to seek medical intervention for worsening liver function.

At block 816, in addition to determining whether the user's glucose levels are greater than a threshold value, therapy management engine 114 monitors the user's glucose time in range. If the user's glucose time in range decreases over time, therapy management engine 114 provides feedback to the user regarding worsening liver function and/or development of diabetes.

Alternatively, returning to block 814, therapy management engine 114 determines if the user's glucose levels are decreasing. Based on this determination, therapy management engine 114 proceeds to block 828 to determine whether the user's glucose levels are below a threshold value (e.g., less than 70 mg/dL). If the user's glucose levels are below a threshold, therapy management engine 114 proceeds to block 830. At block 830, therapy management engine 114 provides feedback to the user to seek medical intervention for worsening liver function and/or risk of developing hepatic encephalopathy. Alternatively, if the user's glucose levels are not below a threshold, therapy management engine 114 proceeds to block 832. At block 832, therapy management engine 114 returns to block 812 and continue monitoring the user's glucose levels over time.

Finally, returning to block 814, if the user's glucose levels are not increasing or decreasing (e.g., remaining stable), therapy management engine 114 returns to block 812 to continue monitoring glucose levels over time.

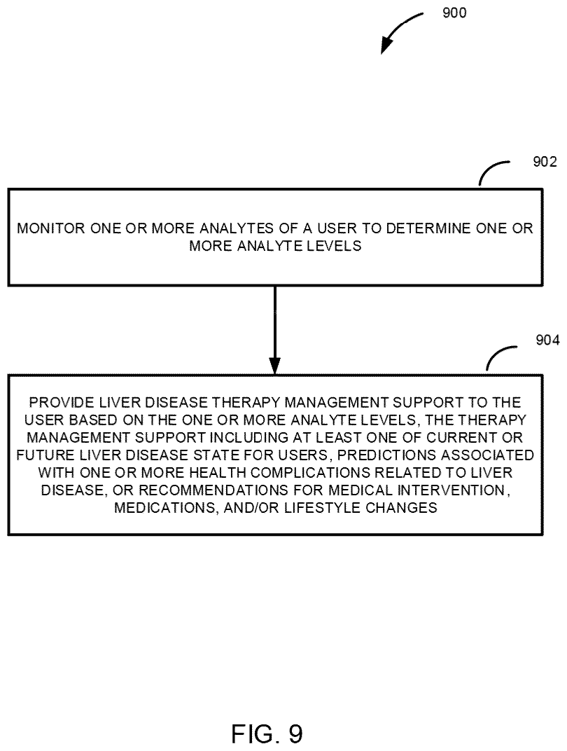

FIG. 9 is a flow diagram depicting an example method 900 for providing liver disease therapy management and providing guidance to improve the user's liver disease state and/or improve various health complications. Method 900 can be performed by therapy management system 100 to collect data, including for example, analyte data generated by a continuous analyte monitoring system 104, user information, and non-analyte sensor data mentioned above, to provide (1) liver disease therapy management and (2) therapy guidance for medical intervention, medications, and/or lifestyle changes to improve the user's liver disease state and/or improve various health complications.

Method 900 begins at block 902 by continuous analyte monitoring system 140 monitoring one or more analytes of a user to determine one or more analyte levels. For example, continuous analyte monitoring system 104 can include a continuous potassium, lactate, ammonia, sodium, and/or glucose sensor 202 to measure the user's analyte levels, such as potassium, lactate, ammonia, sodium, and/or glucose levels. Therapy management engine 114 can then receive measured analyte levels from continuous analyte monitoring system 104. Further, therapy management engine 114 can receive data from user inputs. The user inputs can be received in a variety of ways as described herein.

At block 904, therapy management engine 114 provides liver disease therapy management support to the user based on the one or more analyte levels. As described above, the therapy management output indicates at least one of (1) a current or future liver disease state of a user, (2) predictions associated with one or more health complications related to liver disease, or (3) medical intervention, medications, and/or lifestyle changes.

The liver disease therapy management guidance is provided by therapy management engine 114 based on one or more of the steps of the methods described with reference to FIGS. 4-8.

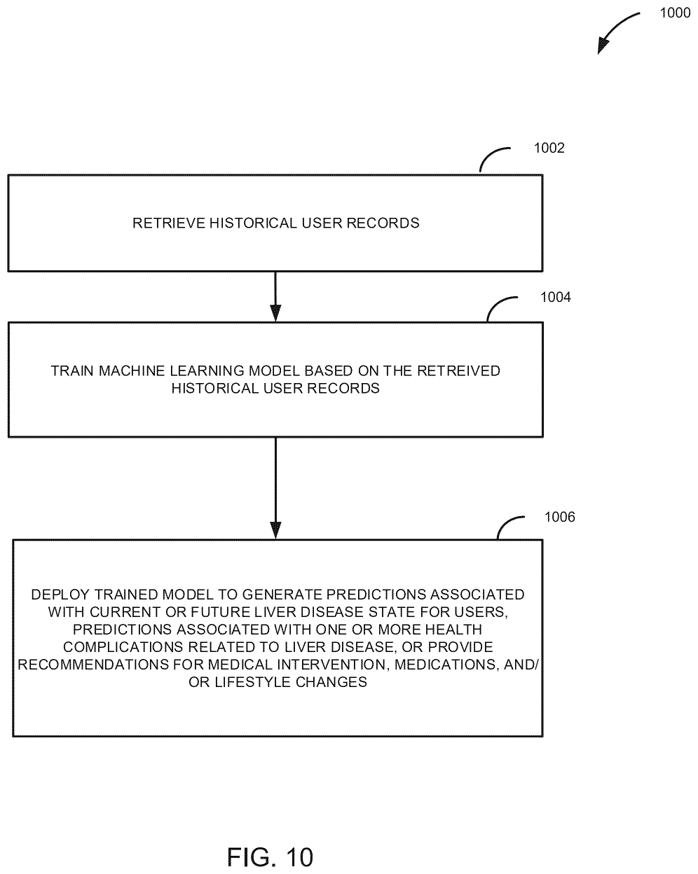

In certain embodiments, machine learning models deployed by therapy management engine 114 include one or more models trained by training system 140, as illustrated in FIG. 1. FIG. 10 describes in further detail techniques for training the machine learning model(s) deployed by therapy management engine 114 for predicting a current or future liver disease state, predicting presence of one or more health complications related to liver disease, and/or providing therapy guidance for medical intervention, medications, and/or lifestyle changes to modify the analyte state of the patient. The changes and/or recommendations can then be further adjusted as necessary based on continuous analyte measurements taken at a future time and/or analyte metrics determined at a time following the recommendation and/or guidance. Changes in the user's continuous analyte measurements and/or analyte metrics can indicate the effect of the therapy guidance and the therapy management engine 114 can update the user's therapy guidance, if needed.

FIG. 10 is a flow diagram depicting a method 900 for training machine learning models to predict a user's current or future liver disease state, predict the presence of one or more health complications related to liver disease, and/or provide recommendations to a user based on disease state. In certain embodiments, the method 500 is used to train models for predicting a current or future liver disease state and/or the presence of one or more health complications, as illustrated in FIG. 1.

Method 1000 begins, at block 1002, by training system, such as training system 140 illustrated in FIG. 1, retrieving data from historical records database, such as historical records database 112 illustrated in FIG. 1. As mentioned herein, historical records database 112 may provide a repository of up-to-date information and historical information for users of a continuous analyte monitoring system and connected mobile health application, such as users of continuous analyte monitoring system 104 and application 106 illustrated in FIG. 1, as well as data for one or more users who are not, or were not previously, users of continuous analyte monitoring system 104 and/or application 106. In certain embodiments, historical records database 112 includes one or more data sets of historical users who are healthy users, users with liver disease, and/or users with one or more health complications related to liver disease.

Retrieval of data from historical records database 112 by training system 140, at block 1002, can include the retrieval of all, or any subset of, information maintained by historical records database 112. For example, where historical records database 112 stores information for 100,000 users (e.g., non-users and users of continuous analyte monitoring system 104 and application 106), data retrieved by training system 140 to train one or more machine learning models includes information for all 100,000 users or only a subset of the data for those users, e.g., data associated with only 50,000 users or only data from the last ten years.

As an illustrative example, integrating with on premises or cloud based medical record databases through Fast Healthcare Interoperability Resources (FHIR), web application programming interfaces (APIs), Health Level 7 (HL7), and/or other computer interface language can enable aggregation of healthcare historical records for baseline assessment in addition to the aggregation of de-identifiable user data from a cloud based repository. Similarly, when integrating into the medical record databases, the integration can be accomplished by directly interfacing with the electronic medical record system or through one or more intermediary systems (e.g., an interface engine, etc.).

As an illustrative example, at block 1002, training system 140 can retrieve information for 100,000 users with various disease states (e.g., healthy user, user with liver disease, and/or a user with one or more health complications) stored in historical records database 112 to train a model to predict a current or future liver disease state of a user and provide recommendations to the user. Each of the 100,000 users can have a corresponding data record (e.g., based on their corresponding user profile), stored in historical records database 112. Each user profile 118 can include information, such as information discussed with respect to FIG. 3.

The training system 140 then uses information in each of the records to train an artificial intelligence or ML model (for simplicity referred to as “ML model” herein). Examples of types of information included in a user's user profile were provided above. The information in each of these records can be featurized (e.g., manually or by training system 140), resulting in features that can be used as input features for training the ML model. For example, a user record can include or be used to generate features related to the user's demographic information (e.g., an age of a user, a gender of the user, etc.), analyte information, such as ammonia, lactate, potassium, sodium, and glucose metrics, non-analyte information, and/or any other data points in the user record (e.g., inputs 130, metrics 132, etc.). Features used to train the machine learning model(s) can vary in different embodiments.

In certain embodiments, each historical user record retrieved from historical records database 112 is further associated with a label indicating a medical diagnosis of the user (e.g., a healthy user, a user with liver disease, and/or a user with one or more health complications), current liver disease state or state of one or more health complications, etc. What the record is labeled with would depend on what the model is being trained to predict.

At block 1004, method 1000 continues by training system 140 training one or more machine learning models based on the features and labels associated with the historical user records. In some embodiments, the training server does so by providing the features as input into a model. This model can be a new model initialized with random weights and parameters, or can be partially or fully pre-trained (e.g., based on prior training rounds). Based on the input features, the model-in-training generates some output. In certain embodiments, the output can include a current or future liver disease state, a diagnosis of one or more health complications, and/or recommendations for medical intervention, medications, and/or lifestyle changes to improve the user's liver disease state or improve the state of one or more health complications, or similar outputs. Note that the output could be in the form of a classification, a recommendation, and/or other types of output.

In certain embodiments, training system 140 compares this generated output with the actual label associated with the corresponding historical user record to compute a loss based on the difference between the actual result and the generated result. This loss is then used to refine one or more internal weights and parameters of the model (e.g., via backpropagation) such that the model learns to predict a current or future liver disease state, and/or provide recommendations for treatment, medications, and/or lifestyle changes to improve the user's liver disease state more accurately.

One of a variety of machine learning algorithms can be used for training the model(s) described above. For example, one of a supervised learning algorithm, a neural network algorithm, a deep neural network algorithm, a deep learning algorithm, etc. can be used.

At block 1006, training system 140 deploys the trained model(s) to make predictions associated with current or future liver disease state or one or more health complications during runtime. In some embodiments, this includes transmitting some indication of the trained model(s) (e.g., a weights vector) that can be used to instantiate the model(s) on another device. For example, training system 140 can transmit the weights of the trained model(s) to therapy management engine 114, which could execute on display device 107, etc. The model(s) can then be used to determine, in real-time, a current or future liver disease state or one or more health complications of a user using application 106, and/or make other types of recommendations discussed above. In certain embodiments, the training system 140 can continue to train the model(s) in an “online” manner by using input features and labels associated with new user records.

Further, similar methods for training illustrated in FIG. 10 using historical user records can also be used to train models using user-specific records to create more personalized models for making predictions associated with a current or future liver disease state and/or one or more health complications related to liver disease. For example, a model trained using historical user records that is deployed for a particular user, can be further re-trained after deployment. For example, the model can be re-trained after the model is deployed for a specific user to create a more personalized model for the user. The more personalized model can be able to more accurately make predictions on disease state of the user, provide feedback on the development of liver disease and/or health complications related to liver disease, and/or provide diet and lifestyle recommendations based on the user's own data (as opposed to only historical user record data), including the user's own inputs 130 and metrics 132.

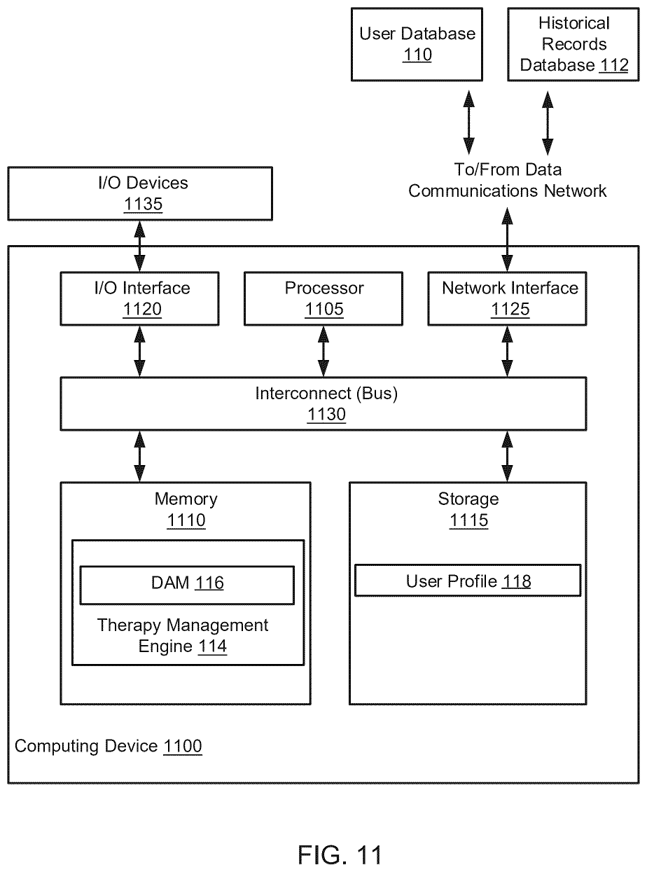

FIG. 11 is a block diagram depicting a computing device 1100 configured to execute a therapy management engine (e.g., therapy management engine 114), according to certain embodiments disclosed herein. Although depicted as a single physical device, in embodiments, computing device 1100 is implemented using virtual device(s), and/or across a number of devices, such as in a cloud environment. As illustrated, computing device 1100 includes a processor 1105, memory 1110, storage 1015, a network interface 1125, and one or more I/O interfaces 1120. In the illustrated embodiment, processor 1105 retrieves and executes programming instructions stored in memory 1110, as well as stores and retrieves application data residing in storage 1115. Processor 1105 is generally representative of a single CPU and/or GPU, multiple CPUs and/or GPUs, a single CPU and/or GPU having multiple processing cores, and the like. Memory 1110 is generally included to be representative of a random-access memory. Storage 1115 can be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, input and output (I/O) devices 1135 (such as keyboards, monitors, etc.) can be connected via the I/O interface(s) 1020. Further, via network interface 1125, computing device 1100 can be communicatively coupled with one or more other devices and components, such as user database 110. In certain embodiments, computing device 1100 is communicatively coupled with other devices via a network, which can include the Internet, local network(s), and the like. The network can include wired connections, wireless connections, or a combination of wired and wireless connections. As illustrated, processor 1005, memory 1010, storage 1115, network interface(s) 1125, and I/O interface(s) 1120 are communicatively coupled by one or more interconnects 1130. In certain embodiments, computing device 1100 is representative of display device 107 associated with the user. In certain embodiments, as discussed above, the display device 107 can include the user's laptop, computer, smartphone, and the like. In another embodiment, computing device 1100 is a server executing in a cloud environment.

In the illustrated embodiment, storage 1115 includes user profile 118. Memory 1110 includes therapy management engine 114, which itself includes DAM 116.

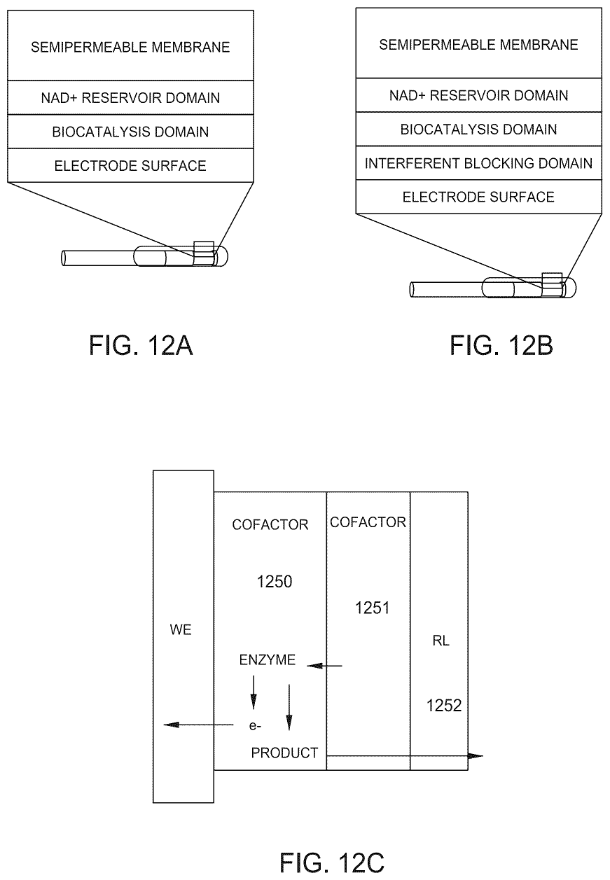

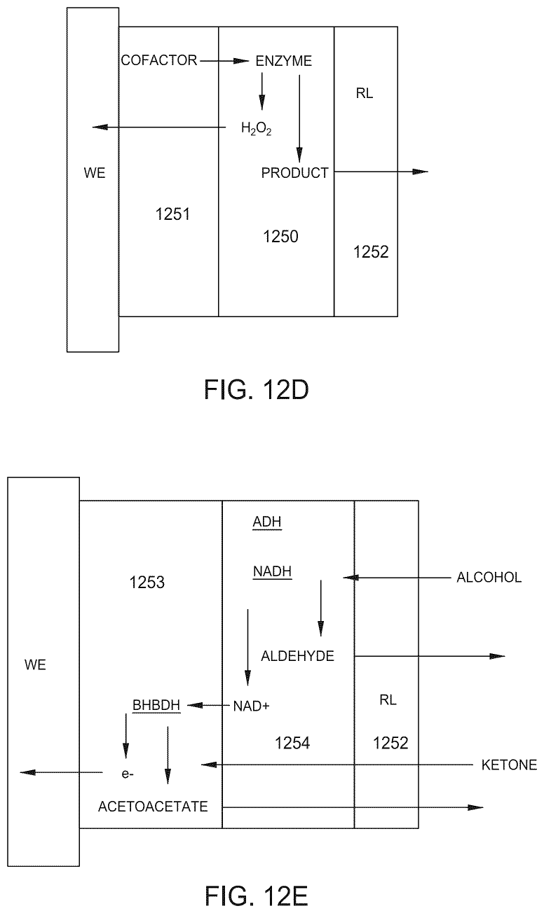

As described above, continuous analyte monitoring system 104, described in relation to FIG. 1, can be a multi-analyte sensor system including a multi-analyte sensor. FIGS. 12A-16D describe example multi-analyte sensors used to measure multiple analytes.

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases may refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing (detecting) element.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In one example, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In one example, such biosensors and/or sensors are capable of providing specific quantitative, semi-quantitative, qualitative, semi qualitative analytical signals using a biological recognition element combined with a transducing (detecting) element.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms can provide specific quantitative, semi-quantitative, qualitative, semi qualitative analytical signals using a biological recognition element combined with a transducing (detecting) element.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between patient tissue and an implantable device. The terms “biointerface” and “bioprotective” are used interchangeably herein.

The term “cofactor” as used herein is a broad term, and is to be given its ordinary and customary meaning to a person of ordinary skill in the art (and is not to be limited to a special or customized meaning), and refers without limitation to one or more substances whose presence contributes to or is required for analyte-related activity of an enzyme. Analyte-related activity can include, but is not limited to, any one of or a combination of binding, electron transfer, and chemical transformation. Cofactors are inclusive of coenzymes, non-protein chemical compounds, metal ions and/or metal organic complexes. Coenzymes are inclusive of prosthetic groups and co-substrates.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrases “continuous analyte sensing” and “continuous multi-analyte sensing” as used herein are broad phrases, and are to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7,10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks.

The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected,” “operably linked”, and “operably coupled” as used herein may refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e., “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components. covalently, electrostatically, ionically associated with, or physically entrapped, or absorbed.

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of a membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In one example this reaction is faradaic and results in charge transfer between the surface and its environment. In one example, hydrogen peroxide produced by an enzyme-catalyzed reaction of an analyte being oxidized on the surface results in a measurable electronic current. For example, in the detection of glucose, glucose oxidase produces hydrogen peroxide (H₂O₂) as a byproduct. The H₂O₂ reacts with the surface of the working electrode to produce two protons (2H⁺), two electrons (2e⁻) and one molecule of oxygen (O₂), which produces the electronic current being detected. In a counter electrode, a reducible species, for example, O₂ is reduced at the electrode surface so as to balance the current generated by the working electrode.

The term “electrolysis” as used herein is a broad term, and is to be given its ordinary and customary meaning to a person of ordinary skill in the art (and is not to be limited to a special or customized meeting), and refers without limitation to electrooxidation or electroreduction (collectively, “redox”) of a compound, either directly or indirectly, by one or more enzymes, cofactors, or mediators.

The terms “indwelling,” “in dwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), or transcutaneously (i.e. penetrating, entering, or passing through intact skin), which may result in a sensor that has an in vivo portion and an ex vivo portion. The term “indwelling” also encompasses an object which is configured to be inserted subcutaneously, intracutaneously, or transcutaneously, whether or not it has been inserted as such.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are compounds which produce a signal that is not analyte-specific due to a reaction on an electrochemically active surface.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a patient.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a patient.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In one example the mediator accepts electrons from, or transfer electrons to, one or more enzymes or cofactors, and/or exchanges electrons with the sensor system electrodes. In one example, mediators are transition-metal coordinated organic molecules which are capable of reversible oxidation and reduction reactions. In other examples, mediators may be organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between patient tissue and the implantable device, modulation of patient tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In one example, the membrane system comprises an enzyme, which enables an analyte reaction to occur whereby a concentration of the analyte can be measured.

The term “planar” as used herein is to be interpreted broadly to describe sensor architecture having a substrate including at least a first surface and an opposing second surface, and for example, comprising a plurality of elements arranged on one or more surfaces or edges of the substrate. The plurality of elements can include conductive or insulating layers or elements configured to operate as a circuit. The plurality of elements may or may not be electrically or otherwise coupled. In one example, planar includes one or more edges separating the opposed surfaces.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme domain or layer. If the sensor is deemed to be the point of reference and the enzyme domain is positioned nearer to the sensor than the biointerface layer, then the enzyme domain is more proximal to the sensor than the biointerface layer.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms are capable of providing specific quantitative, semi-quantitative, qualitative, semi qualitative analytical signals using a biological recognition element combined with a transducing and/or detecting element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, ketone, lactate, potassium, etc., in the biological sample.

In one example, the sensing region or sensing portion can comprise at least a portion of a conductive substrate or at least a portion of a conductive surface, for example, a wire (coaxial) or conductive trace or a substantially planar substrate including substantially planar trace(s), and a membrane. In one example, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) including mutant or fusion protein thereof, or aptamers having one or more analyte binding regions, each region capable of specifically and reversibly binding to at least one analyte. Alterations of the aptamer or mutations of the PBP can contribute to or alter one or more of the binding constants, long-term stability of the protein, including thermal stability, to bind the protein to a special encapsulation matrix, membrane or polymer, or to attach a detectable reporter group or “label” to indicate a change in the binding region or transduce a signal corresponding to the one or more analytes present in the biological fluid. Specific examples of changes in the binding region include, but are not limited to, hydrophobic/hydrophilic environmental changes, three-dimensional conformational changes, changes in the orientation of amino/nucleic acid side chains in the binding region of proteins, and redox states of the binding region. Such changes to the binding region provide for transduction of a detectable signal corresponding to the one or more analytes present in the biological fluid.

In one example, the sensing region determines the selectivity among one or more analytes, so that only the analyte which has to be measured leads to (transduces) a detectable signal. The selection can be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoAmps of current for every 1 mg/dL of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The terms “transducing” or “transduction” and their grammatical equivalents as are used herein are broad terms, and are to be given their ordinary and customary meaning to a person of ordinary skill in the art (and is not to be limited to a special or customized meaning), and refer without limitation to optical, electrical, electrochemical, acoustical/mechanical, or colorimetrical technologies and methods. Electrochemical properties include current and/or voltage, inductance, capacitance, impedance, transconductance, and potential. Optical properties include absorbance, fluorescence/phosphorescence, fluorescence/phosphorescence decay rate, wavelength shift, dual wave phase modulation, bio/chemiluminescence, reflectance, light scattering, and refractive index. For example, the sensing region transduces the recognition of analytes into a semi-quantitative or quantitative signal.

As used herein, the phrase “transducing element” as used herein is a broad phrase, and are to be given their ordinary and customary meaning to a person of ordinary skill in the art (and is not to be limited to a special or customized meaning), and refers without limitation to analyte recognition moieties capable of facilitating, directly or indirectly, with detectable signal transduction corresponding to the presence and/or concentration of the recognized analyte. In one example, a transducing element is one or more enzymes, one or more aptamers, one or more ionophores, one or more capture antibodies, one or more proteins, one or more biological cells, one or more oligonucleotides, and/or one or more DNA or RNA moieties. Transcutaneous continuous multi-analyte sensors can be used in vivo over various lengths of time. The continuous multi-analyte sensor systems discussed herein can be transcutaneous devices, in that a portion of the device can be inserted through the patient's skin and into the underlying soft tissue while a portion of the device remains on the surface of the patient's skin. In one aspect, in order to overcome the problems associated with noise or other sensor function in the short-term, one example employs materials that promote formation of a fluid pocket around the sensor, for example architectures such as a porous biointerface membrane or matrices that create a space between the sensor and the surrounding tissue. In some examples, a sensor is provided with a spacer adapted to provide a fluid pocket between the sensor and the patient's tissue. It is believed that this spacer, for example a biointerface material, matrix, structure, and the like as described in more detail elsewhere herein, provides for oxygen and/or glucose transport to the sensor.

### Membrane Systems

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels for individuals having diabetes. In some examples, the analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572, 5,1264,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, spraying, electrodepositing, dipping, brush coating, film coating, drop-let coating, and the like). Additional steps can be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (μm), or less, to about 1, 2, 3, 4, 5, 6, 7, 8, 9, 10, 11, 12, 13, 14, 15, or 16 μm is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl) carbodiimide (EDC)) and cured at a moderate temperature of about 50° C.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro) chemical reactions involved in sensing, but forms chemical bonds with the (electro) chemically active components of the membrane. In some examples these bonds are covalent. In one example, a tether can be formed in solution prior to one or more interlayers of a membrane being formed, where the tether bonds two (electro) chemically active components directly to one another or alternately, the tether(s) bond (electro) chemically active component(s) to polymeric backbone structures. In another example, (electro) chemically active components are comixed along with crosslinker(s) with tunable lengths (and optionally polymers) and the tethering reaction occurs as in situ crosslinking. Tethering can be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as spraying, dipping, casting, electrospinning, vapor deposition, spin coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEGDE), or dicumyl peroxide (DCP). In one example, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spraying, self-assembling monolayers (SAMs), painting, dip coating, vapor depositing, molding, 3-D printing, lithographic techniques (e.g., photolithograph), micro- and nano-pipetting printing techniques, silk-screen printing, etc.). The mixture can then be cured under high temperature (e.g., from about 30° C. to about 150° C.). Other suitable curing methods can include ultraviolet, e-beam, or gamma radiation, for example.

In some circumstances, using continuous multianalyte monitoring systems including sensor(s) configured with bioprotective and/or drug releasing membranes, it is believed that foreign body response is the dominant event surrounding extended implantation of an implanted device and can be managed or manipulated to support rather than hinder or block analyte transport. In another aspect, in order to extend the lifetime of the sensor, one example employs materials that promote vascularized tissue ingrowth, for example within a porous biointerface membrane. For example, tissue in-growth into a porous biointerface material surrounding a sensor can promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous bioprotective materials that surround the sensor and promote ingrowth of tissue and microvasculature over time.

Accordingly, a sensor as discussed in examples herein can include a biointerface layer. The biointerface layer, like the drug releasing layer, can include, but is not limited to, for example, porous biointerface materials including a solid portion and interconnected cavities, all of which are described in more detail elsewhere herein. The biointerface layer can be employed to improve sensor function in the long term (e.g., after tissue ingrowth).

Accordingly, a sensor as discussed in examples herein can include a drug releasing membrane at least partially functioning as or in combination with a biointerface membrane. The drug releasing membrane can include, for example, materials including a hard-soft segment polymer with hydrophilic and optionally hydrophobic domains, all of which are described in more detail elsewhere herein, can be employed to improve sensor function in the long term (e.g., after tissue ingrowth). In one example, the materials including a hard-soft segment polymer with hydrophilic and optionally hydrophobic domains are configured to release a combination of a derivative form of dexamethasone or dexamethasone acetate with dexamethasone such that one or more different rates of release of the anti-inflammatory is achieved and the useful life of the sensor is extended. Other suitable drug releasing membranes of the present disclosure can be selected from silicone polymers, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), poly vinyl acetate, ethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(l-lysine), poly(L-lactic acid), hydroxyethylmetharcrylate and copolymers and blends thereof, and hydroxyapeptite and copolymers and blends thereof.

### Exemplary Multi-Analyte Sensor Membrane Configurations

Continuous multi-analyte sensors with various membrane configurations suitable for facilitating signal transduction corresponding to analyte concentrations, either simultaneously, intermittently, and/or sequentially are provided. In one example, such sensors can be configured using a signal transducer, comprising one or more transducing elements (“TL”). Such continuous multi-analyte sensor can employ various transducing means, for example, amperometry, voltametric, potentiometry, and impedimetric methods, among other techniques.

In one example, the transducing element comprises one or more membranes that can comprise one or more layers and or domains, each of the one or more layers or domains can independently comprise one or more signal transducers, e.g., enzymes, RNA, DNA, aptamers, binding proteins, etc. As used herein, transducing elements includes enzymes, ionophores, RNA, DNA, aptamers, binding proteins and are used interchangeably.

In one example, the transducing element is present in one or more membranes, layers, or domains formed over a sensing region. In one example, such sensors can be configured using one or more enzyme domains, e.g., membrane domains including enzyme domains, also referred to as EZ layers (“EZLs”), each enzyme domain can comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

In one example, the continuous multi-analyte sensor uses one or more of the following analyte-substrate/enzyme pairs: for example, sarcosine oxidase in combination with creatinine amidohydrolase, creatine amidohydrolase being employed for the sensing of creatinine. Other examples of analytes/oxidase enzyme combinations that can be used in the sensing region include, for example, alcohol/alcohol oxidase, cholesterol/cholesterol oxidase, glactose: galactose/galactose oxidase, choline/choline oxidase, glutamate/glutamate oxidase, glycerol/glycerol-3phosphate oxidase (or glycerol oxidase), bilirubin/bilirubin oxidase, ascorbic/ascorbic acid oxidase, uric acid/uric acid oxidase, pyruvate/pyruvate oxidase, hypoxanthine: xanthine/xanthine oxidase, glucose/glucose oxidase, lactate/lactate oxidase, L-amino acid oxidase, and glycine/sarcosine oxidase. Other analyte-substrate/enzyme pairs can be used, including such analyte-substrate/enzyme pairs that comprise genetically altered enzymes, immobilized enzymes, mediator-wired enzymes, dimerized and/or fusion enzymes.

### NAD Based Multi-Analyte Sensor Platform

Nicotinamide adenine dinucleotide (NAD(P)⁺/NAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD(P)+) and reduced form (NAD(P)H) (H=hydrogen). The reaction of NAD+ and NADH is reversible, thus, the coenzyme can continuously cycle between the NAD(P)⁺/and NAD(P)H forms essentially without being consumed.

In one example, one or more enzyme domains of the sensing region of the presently disclosed continuous multi-analyte sensor device comprise an amount of NAD+ or NADH for providing transduction of a detectable signal corresponding to the presence or concentration of one or more analytes. In one example, one or more enzyme domains of the sensing region of the presently disclosed continuous multi-analyte sensor device comprise an excess amount of NAD+ or NADH for providing extended transduction of a detectable signal corresponding to the presence or concentration of one or more analytes.

In one example, NAD, NADH, NAD+, NAD(P)+, ATP, flavin adenine dinucleotide (FAD), magnesium (Mg++), pyrroloquinoline quinone (PQQ), and functionalized derivatives thereof can be used in combination with one or more enzymes in the continuous multi-analyte sensor device. In one example, NAD, NADH, NAD+, NAD(P)+, ATP, flavin adenine dinucleotide (FAD), magnesium (Mg++), pyrroloquinoline quinone (PQQ), and functionalized derivatives are incorporated in the sensing region. In one example, NAD, NADH, NAD+, NAD(P)+, ATP, flavin adenine dinucleotide (FAD), magnesium (Mg++), pyrroloquinoline quinone (PQQ), and functionalized derivatives are dispersed or distributed in one or more membranes or domains of the sensing region.

In one aspect of the present disclosure, continuous sensing of one or more or two or more analytes using NAD+ dependent enzymes is provided in one or more membranes or domains of the sensing region. In one example, the membrane or domain provides retention and stable recycling of NAD+ as well as mechanisms for transducing NADH oxidation or NAD+ reduction into measurable current with amperometry. In one example, described below, continuous, sensing of multi-analytes, either reversibly bound or at least one of which are oxidized or reduced by NAD+ dependent enzymes, for example, ketones (beta-hydroxybutyrate dehydrogenase), glycerol (glycerol dehydrogenase), cortisol (11β-hydroxysteroid dehydrogenase), glucose (glucose dehydrogenase), alcohol (alcohol dehydrogenase), aldehydes (aldehyde dehydrogenase), and lactate (lactate dehydrogenase) is provided. In other examples, described below, membranes are provided that enable the continuous, on-body sensing of multiple analytes which utilize FAD-dependent dehydrogenases, such as fatty acids (Acyl-CoA dehydrogenase).

Exemplary configurations of one or more membranes or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 12A. With reference to FIG. 12B, one or more optional layers can be positioned between the electrode surface and the one or more enzyme domains. For example, one or more interference domains (also referred to as “interferent blocking layer”) can be used to reduce or eliminate signal contribution from undesirable species present, or one or more electrodes (not shown) can used to assist with wetting, system equilibrium, and/or start up. As shown in FIGS. 12A-12B, one or more of the membranes provides a NAD+ reservoir domain providing a reservoir for NAD+. In one example, one or more interferent blocking membranes is used, and potentiostat is utilized to measure H2O2 production or O2 consumption of an enzyme such as or similar to NADH oxidase, the NAD+ reservoir and enzyme domain positions can be switched, to facilitate better consumption and slower unnecessary outward diffusion of excess NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein.

In one example, one or more mediators that are optimal for NADH oxidation are incorporated in the one or more electrode domains or enzyme domains. In one example, organic mediators, such as phenanthroline dione, or nitrosoanilines are used. In another example, metallo-organic mediators, such as ruthenium-phenanthroline-dione or osmium (bpy)₂Cl, polymers containing covalently coupled organic mediators or organometallic coordinated mediators polymers for example polyvinylimidizole-Os(bpy)₂Cl, or polyvinylpyridine-organometallic coordinated mediators (including ruthenium-phenanthroline dione) are used. Other mediators can be used as discussed further below.

In humans, serum levels of beta-hydroxybutyrate (BHB) are usually in the low micromolar range but can rise up to about 6-8 mM. Serum levels of BHB can reach 1-2 mM after intense exercise or consistent levels above 2 mM are reached with a ketogenic diet that is almost devoid of carbohydrates. Other ketones are present in serum, such as acetoacetate and acetone, however, most of the dynamic range in ketone levels is in the form of BHB. Thus, monitoring of BHB, e.g., continuous monitoring is useful for providing health information to a patient or health care provider.

Another example of a continuous ketone analyte detection configuration employing electrode-associated mediator-coupled diaphorase/NAD+/dehydrogenase is depicted below:

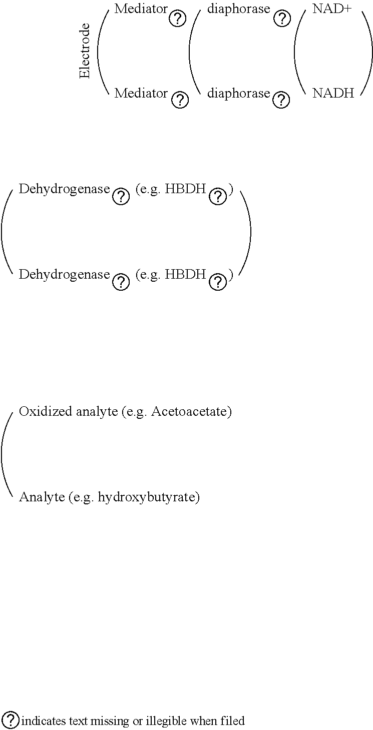

In one example, the diaphorase is electrically coupled to the electrode with organometallic coordinated mediator polymer. In another example, the diaphorase is covalently coupled to the electrode with an organometallic coordinated mediator polymer. Alternatively, multiple enzyme domains can be used in an enzyme layer, for example, separating the electrode-associated diaphorase (closest to the electrode surface) from the more distal adjacent NAD+ or the dehydrogenase enzyme, to essentially decouple NADH oxidation from analyte (ketone) oxidation. Alternatively, NAD+ can be more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, NAD+ can be covalently coupled to an aspect of the enzyme domain having a higher molecular weight than the NAD+ which can improve a stability profile of the NAD+, improving the ability to retain and/or immobilize the NAD+ in the enzyme domain. For example, dextran-NAD.

In one example, the sensing region comprises one or more NADH: acceptor oxidoreductases and one or more NAD-dependent dehydrogenases. In one example, sensing region comprises one or more NADH: acceptor oxidoreductases and one or more NAD(P)-dependent dehydrogenases with NAD(P)+ or NAD(P)H as cofactors present in sensing region. In one example, the sensing region comprises an amount of diaphorase.

In one example, a ketone sensing configuration suitable for combination with another analyte sensing configuration is provided. Thus, an EZL layer of about 1-20 um thick is prepared by presenting a EZL solution composition in 10 mM HEPES in water having about 20 uL 500 mg/mL HBDH, about 20 uL [500 mg/mL NAD(P)H, 200 mg/mL polyethylene glycol-diglycol ether (PEG-DGE) of about 400 MW], about 20 uL 500 mg/mL diaphorase, about 40 uL 250 mg/mL poly vinyl imidazole-osmium bis(2,2′-bipyridine)chloride (PVI-Os(bpy)2Cl) to a substrate such as a working electrode, so as to provide, after drying, about 15-40% by weight HBDH, about 5-30% diaphorase about 5-30% NAD(P)H, about 10-50% PVI-Os(bpy)2Cl and about 1-12% PEG-DGE (400 MW). The substrates discussed herein that can include working electrodes may be formed from gold, platinum, palladium, rhodium, iridium, titanium, tantalum, chromium, and/or alloys or combinations thereof, or carbon (e.g., graphite, glassy carbon, carbon nanotubes, graphene, or doped diamond, as well combinations thereof.

To the above enzyme domain was contacted a resistance domain, also referred to as a resistance layer (“RL”). In one example, the RL comprises about 55-100% PVP, and about 0.1-45% PEG-DGE. In another example, the RL comprises about 75-100% PVP, and about 0.3-25% PEG-DGE. In yet another example, the RL comprises about 85-100% PVP, and about 0.5-15% PEG-DGE. In yet another example, the RL comprises essentially 100% PVP.

The exemplary continuous ketone sensor as depicted in FIGS. 12A-12B comprising NAD(P)H reservoir domain is configured so that NAD(P)H is not rate-limiting in any of the enzyme domains of the sensing region. In one example, the loading of NAD(P)H in the NAD(P)H reservoir domain is greater than about 20%, 30%, 40% or 50% w/w. The one or more of the membranes or portions of one or more membrane domains (hereinafter also referred to as “membranes”) can also contain a polymer or protein binder, such as zwitterionic polyurethane, and/or albumin. Alternatively, in addition to NAD(P)H, the membrane can contain one or more analyte specific enzymes (e.g. HBDH, glycerol dehydrogenase, etc.), so that optionally, the NAD(P)H reservoir membrane also provides a catalytic function. In one example, the NAD(P)H is dispersed or distributed in or with a polymer (or protein), and can be crosslinked to an extent that still allows adequate enzyme/cofactor functionality and/or reduced NAD(P)H flux within the domain.

In one example, NADH oxidase enzyme alone or in combination with superoxide dismutase (SOD) is used in the one or more membranes of the sensing region. In one example, an amount of superoxide dismutase (SOD) is used that is capable of scavenging some or most of one or more free radicals generated by NADH oxidase. In one example, NADH oxidase enzyme alone or in combination with superoxide dismutase (SOD) is used in combination with NAD(P)H and/or a functionalized polymer with NAD(P)H immobilized onto the polymer from a C6 terminal amine in the one or more membranes of the sensing region.

In one example, the NAD(P)H is immobilized to an extent that maintains NAD(P)H catalytic functionality. In one example, dimerized NAD(P)H is used to entrap NAD(P)H within one or more membranes by crosslinking their respective C6 terminal amine together with appropriate amine-reactive crosslinker such as glutaraldehyde or PEG-DGE.

The aforementioned continuous ketone sensor configurations can be adapted to other analytes or used in combination with other sensor configurations. For example, analyte(s)-dehydrogenase enzyme combinations can be used in any of the membranes of the sensing region include; glycerol (glycerol dehydrogenase); cortisol (11β-hydroxysteroid dehydrogenase); glucose (glucose dehydrogenase); alcohol (alcohol dehydrogenase); aldehydes (aldehyde dehydrogenase); and lactate (lactate dehydrogenase).

In one example, a semipermeable membrane is used in the sensing region or adjacent thereto or adjacent to one or more membranes of the sensing region so as to attenuate the flux of at least one analyte or chemical species. In one example, the semipermeable membrane attenuates the flux of at least one analyte or chemical species so as to provide a linear response from a transduced signal. In another example, the semipermeable membrane prevents or eliminates the flux of NAD(P)H out of the sensing region or any membrane or domain. In one example, the semipermeable membrane can be an ion selective membrane selective for an ion analyte of interest, such as ammonium ion.

In another example, a continuous multi-analyte sensor configuration comprising one or more enzymes and/or at least one cofactor was prepared. In certain embodiments, an enzyme domain 1250 comprising an enzyme (Enzyme) with an amount of cofactor (Cofactor) is positioned proximal to at least a portion of a working electrode (“WE”) surface, where the WE comprises an electrochemically reactive surface. In one example, a second membrane 1251 comprising an amount of cofactor is positioned adjacent the first enzyme domain. The amount of cofactor in the second membrane can provide an excess for the enzyme, e.g., to extend sensor life. One or more resistance domains 1252 (“RL”) are positioned adjacent the second membrane (or can be between the membranes). The RL can be configured to block diffusion of cofactor from the second membrane. Electron transfer from the cofactor to the WE transduces a signal that corresponds directly or indirectly to an analyte concentration.

FIGS. 12C-12D depict an alternative enzyme domain configuration comprising a first membrane 1251 with an amount of cofactor that is positioned more proximal to at least a portion of a WE surface. Enzyme domain 1250 comprising an amount of enzyme is positioned adjacent the first membrane.

In the membrane configurations depicted in FIGS. 12C-12D, production of an electrochemically active species in the enzyme domain diffuses to the WE surface and transduces a signal that corresponds directly or indirectly to an analyte concentration. In some examples, the electrochemically active species comprises hydrogen peroxide. For sensor configurations that include a cofactor, the cofactor from the first layer can diffuse to the enzyme domain to extend sensor life, for example, by regenerating the cofactor. For other sensor configurations, the cofactor can be optionally included to improve performance attributes, such as stability. For example, a continuous ketone sensor can comprise NAD(P)H and a divalent metal cation, such as Mg⁺². One or more resistance domains RL can be positioned adjacent the second membrane (or can be between the layers). The RL can be configured to block diffusion of cofactor from the second membrane and/or interferents from reaching the WE surface. Other configurations can be used in the aforementioned configuration, such as electrode, resistance, bio-interfacing, and drug releasing membranes, layers or domains. In other examples, continuous analyte sensors including one or more cofactors that contribute to sensor performance.

FIG. 12E depicts another continuous multi-analyte membrane configuration, where {beta}-hydroxybutyrate dehydrogenase BHBDH in a first enzyme domain 1253 is positioned proximate to a working electrode WE and second enzyme domain 1254, for example, comprising alcohol dehydrogenase (ADH) and NADH is positioned adjacent the first enzyme domain. One or more resistance domains RL 1252 can be deployed adjacent to the second enzyme domain 1254. In this configuration, the presence of the combination of alcohol and ketone in serum works collectively to provide a transduced signal corresponding to at least one of the analyte concentrations, for example, ketone. Thus, as the NADH present in the more distal second enzyme domain consumes alcohol present in the serum environment, NADH is oxidized to NAD(P)H that diffuses into the first membrane layer to provide electron transfer of the BHBDH catalysis of acetoacetate ketone and transduction of a detectable signal corresponding to the concentration of the ketone. In one example, an enzyme can be configured for reverse catalysis and can create a substrate used for catalysis of another enzyme present, either in the same or different layer or domain. Other configurations can be used in the aforementioned configuration, such as electrode, resistance, bio-interfacing, and drug releasing membranes, layers, or domains. Thus, a first enzyme domain that is more distal from the WE than a second enzyme domain can be configured to generate a cofactor or other element to act as a reactant (and/or a reactant substrate) for the second enzyme domain to detect the one or more target analytes.

### Alcohol Sensor Configurations

In one example, a continuous alcohol (e.g., ethanol) sensor device configuration is provided. In one example, one or more enzyme domains comprising alcohol oxidase (AOX) is provided and the presence and/or amount of alcohol is transduced by creation of hydrogen peroxide, alone or in combination with oxygen consumption or with another substrate-oxidase enzyme system, e.g., glucose-glucose oxidase, in which hydrogen peroxide and or oxygen and/or glucose can be detected and/or measured qualitatively or quantitatively, using amperometry.

In one example, the sensing region for the aforementioned enzyme substrate-oxidase enzyme configurations has one or more enzyme domains comprises one or more electrodes. In one example, the sensing region for the aforementioned enzyme substrate-oxidase enzyme configurations has one or more enzyme domains, with or without the one or more electrodes, further comprises one or interference blocking membranes (e.g. permselective membranes, charge exclusion membranes) to attenuate one or more interferents from diffusing through the membrane to the working electrode. In one example, the sensing region for the aforementioned substrate-oxidase enzyme configurations has one or more enzyme domains, with or without the one or more electrodes, and further comprises one or resistance domains with or without the one or more interference blocking membranes to attenuate one or more analytes or enzyme substrates. In one example, the sensing region for the aforementioned substrate-oxidase enzyme configurations has one or more enzyme domains, with or without the one or more electrodes, one or more resistance domains with or without the one or more interference blocking membranes further comprises one or biointerface membranes and/or drug releasing membranes, independently, to attenuate one or more analytes or enzyme substrates and attenuate the immune response of the patient after insertion.

In one example, the one or more interference blocking membranes are deposited adjacent the working electrode and/or the electrode surface. In one example, the one or interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing thru the sensing region but for oxygen. Such membranes can be used to attenuate alcohol itself as well as attenuate other electrochemically actives species or other analytes that can otherwise interfere by producing a signal if they diffuse to the working electrode.

In one example, the working electrode used comprised platinum and the potential applied was about 0.5 volts.

In one example, sensing oxygen level changes electrochemically, for example in a Clark type electrode setup, or in a different configuration can be carried out, for example, by coating the electrode with one or more membranes of one or more polymers, such as NAFION™ Based on changes of potential, oxygen concentration changes can be recorded, which correlate directly or indirectly with the concentrations of alcohol. When appropriately designed to obey stoichiometric behavior, the presence of a specific concentration of alcohol should cause a commensurate reduction in local oxygen in a direct (linear) relation with the concentration of alcohol. Accordingly, a multi-analyte sensor for both alcohol and oxygen can therefore be provided.

In another example, the above mentioned alcohol sensing configuration can include one or more secondary enzymes that react with a reaction product of the alcohol/alcohol oxidase catalysis, e.g., hydrogen peroxide, and provide for a oxidized form of the secondary enzyme that transduces an alcohol-dependent signal to the WE/RE at a lower potential than without the secondary enzyme. Thus, in one example, the alcohol/alcohol oxidase is used with a reduced form of a peroxidase, for example horse radish peroxidase. The alcohol/alcohol oxidase can be in same or different layer as the peroxidase, or they can be spatially separated distally from the electrode surface, for example, the alcohol/alcohol oxidase being more distal from the electrode surface and the peroxidase being more proximal to the electrode surface, or alternatively, the alcohol/alcohol oxidase being more proximal from the electrode surface and the peroxidase being more distal to the electrode surface. In one example, the alcohol/alcohol oxidase, being more distal from the electrode surface and the peroxidase, further includes any combination of electrode, interference, resistance, and biointerface membranes to optimize signal, durability, reduce drift, or extend end of use duration.

In another example, the above mentioned alcohol sensing configuration can include one or more mediators. In one example, the one or more mediators are present in, on, or about one or more electrodes or electrode surfaces and/or are deposited or otherwise associated with the surface of the working electrode (WE) or reference electrode (RE). In one example, the one or more mediators eliminate or reduce direct oxidation of interfering species that can reach the WE or RE. In one example, the one or more mediators provide a lowering of the operating potential of the WE/RE, for example, from about 0.6V to about 0.3V or less on a platinum electrode, which can reduce or eliminates oxidation of endogenous interfering species. Examples of one or mediators are provided below. Other electrodes, e.g., counter electrodes, can be employed.

In one example, other enzymes or additional components can be added to the polymer mixture(s) that constitute any part of the sensing region to increase the stability of the aforementioned sensor and/or reduce or eliminate the biproducts of the alcohol/alcohol oxidase reaction. Increasing stability includes storage or shelf life and/or operational stability (e.g., retention of enzyme activity during use). For example, byproducts of enzyme reactions can be undesirable for increased shelf life and/or operational stability, and can thus be desirable to reduce or remove. In one example, xanthine oxidase can be used to remove bi-products of one or more enzyme reactions.

In another example, a dehydrogenase enzyme is used with a oxidase for the detection of alcohol alone or in combination with oxygen. Thus, in one example, alcohol dehydrogenase is used to oxidize alcohol to aldehyde in the presence of reduced nicotinamide adenine dinucleotide (NAD(P)H) or reduced nicotinamide adenine dinucleotide phosphate (NAD(P)+). So as to provide a continuous source of NAD(P)H or NAD(P)+, NADH oxidase or NADPH oxidases is used to oxidize the NAD(P)H or NAD(P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor.

In the aforementioned dual enzyme configuration, a signal can be sensed either by: (1) an electrically coupled (e.g., “wired”) alcohol dehydrogenase (ADH), for example, using an electro-active hydrogel polymer comprising one or more mediators; or (2) oxygen electrochemical sensing to measure the oxygen consumption of the NADH oxidase. In an alternative example, the co-factor NAD(P)H or NAD(P)+can be coupled to a polymer, such as dextran, the polymer immobilized in the enzyme domain along with ADH. This provides for retention of the co-factor and availability thereof for the active site of ADH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface is provided. A chemical moiety capable of assisting with electron transfer from the enzyme or cofactor to the electrode surface can be used and includes one or more mediators as described below.

In one example, any one of the aforementioned continuous alcohol sensor configurations are combined with any one of the aforementioned continuous ketone monitoring configurations to provide a continuous multi-analyte sensor device as further described below. In one example a continuous glucose monitoring configuration combined with any one of the aforementioned continuous alcohol sensor configurations and any one of the aforementioned continuous ketone monitoring configurations to provide a continuous multi-analyte sensor device as further described below.

### Uric Acid Sensor Configurations

In another example, a continuous uric acid sensor device configuration is provided. Thus, in one example, uric acid oxidase (UOX) can be included in one or more enzyme domains and positioned adjacent the working electrode surface. The catalysis of the uric acid using UOX, produces hydrogen peroxide which can be detected using, among other techniques, amperometry, voltametric and impedimetric methods. In one example, to reduce or eliminate the interference from direct oxidation of uric acid on the electrode surface, one or more electrode, interference, and/or resistance domains can be deposited on at least a portion of the working electrode surface. Such membranes can be used to attenuate diffusion of uric acid as well as other analytes to the working electrode that can interfere with signal transduction.

In one alternative example, a uric acid continuous sensing device configuration comprises sensing oxygen level changes about the WE surface, e.g., for example, as in a Clark type electrode setup, or the one or more electrodes can comprise, independently, one or more different polymers such as NAFION™, polyzwitterion polymers, or polymeric mediator adjacent at least a portion of the electrode surface. In one example, the electrode surface with the one or more electrode domains provide for operation at a different or lower voltage to measure oxygen. Oxygen level and its changes in can be sensed, recorded, and correlated to the concentration of uric acid based using, for example, using conventional calibration methods.

In one example, alone or in combination with any of the aforementioned configurations, uric acid sensor configurations, so as to lower the potential at the WE for signal transduction of uric acid, one or more coatings can be deposited on the WE surface. The one or more coatings can be deposited or otherwise formed on the WE surface and/or on other coatings formed thereon using various techniques including, but not limited to, dipping, electrodepositing, vapor deposition, spray coating, etc. In one example, the coated WE surface can provide for redox reactions, e.g., of hydrogen peroxide, at lower potentials (as compared to 0.6 V on platinum electrode surface without such a coating. Example of materials that can be coated or annealed onto the WE surface includes, but are not limited to Prussian Blue, Medola Blue, methylene blue, methylene green, methyl viologen, ferrocyanide, ferrocene, cobalt ion, and cobalt phthalocyanine, and the like.

In one example, one or more secondary enzymes, cofactors and/or mediators (electrically coupled or polymeric mediators) can be added to the enzyme domain with UOX to facilitate direct or indirect electron transfer to the WE. In such configurations, for example, regeneration of the initial oxidized form of secondary enzyme is reduced by the WE for signal transduction. In one example, the secondary enzyme is horse radish peroxidase (HRP).

### Choline Sensor Configurations

In one example continuous choline sensor device can be provided, for example, using choline oxidase enzyme that generates hydrogen peroxide with the oxidation of choline. Thus, in one example, at least one enzyme domain comprises choline oxidase (COX) adjacent at least one WE surface, optionally with one or more electrodes and/or interference membranes positioned in between the WE surface and the at least one enzyme domain. The catalysis of the choline using COX results in creation of hydrogen peroxide which can be detectable using, among other techniques, amperometry, voltametric and impedimetric methods.

In one example, the aforementioned continuous choline sensor configuration is combined with any one of the aforementioned continuous alcohol sensor configurations, and continuous uric acid sensor configurations to provide a continuous multi-analyte sensor device as further described below. This continuous multi-analyte sensor device can further include continuous glucose monitoring capability. Other membranes can be used in the aforementioned continuous choline sensor configuration, such as electrode, resistance, bio-interfacing, and drug releasing membranes.

### Cholesterol Sensor Configurations

In one example, continuous cholesterol sensor configurations can be made using cholesterol oxidase (CHOX), in a manner similar to previously described sensors. Thus, one or more enzyme domains comprising CHOX can be positioned adjacent at least one WE surface. The catalysis of free cholesterol using CHOX results in creation of hydrogen peroxide which can be detectable using, among other techniques, amperometry, voltametric and impedimetric methods.

An exemplary cholesterol sensor configuration using a platinum WE, where at least one interference membrane is positioned adjacent at least one WE surface, over which there is at least one enzyme domain comprising CHOX, over which is positioned at least one resistance domain to control diffusional characteristics was prepared.

The method described above and the cholesterol sensors described can measure free cholesterol, however, with modification, the configuration can measure more types of cholesterol as well as total cholesterol concentration. Measuring different types of cholesterol and total cholesterol is important, since due to low solubility of cholesterol in water significant amount of cholesterol is in unmodified and esterified forms. Thus, in one example, a total cholesterol sample is provided where a secondary enzyme is introduced into the at least one enzyme domain, for example, to provide the combination of cholesterol esterase with CHOX Cholesteryl ester, which essentially represents total cholesterols can be measured indirectly from signals transduced from cholesterol present and formed by the esterase.

In one example, the aforementioned continuous (total) cholesterol sensor configuration is combined with any one of the aforementioned continuous alcohol sensor configurations and/or continuous uric acid sensor configurations to provide a continuous multi-analyte sensor system as further described below. This continuous multi-analyte sensor device can further include continuous glucose monitoring capability. Other membrane configurations can be used in the aforementioned continuous cholesterol sensor configuration, such as one or more electrode domains, resistance domains, bio-interfacing domains, and drug releasing membranes.

### Bilirubin Sensor and Ascorbic Acid Sensor Configurations

In one example, continuous bilirubin and ascorbic acid sensors are provided. These sensors can employ bilirubin oxidase and ascorbate oxidase, respectively. However, unlike some oxidoreductase enzymes, the final product of the catalysis of analytes of bilirubin oxidase and ascorbate oxidase is water instead of hydrogen peroxide. Therefore, redox detection of hydrogen peroxide to correlate with bilirubin or ascorbic acid is not possible. However, these oxidase enzymes still consume oxygen for the catalysis, and the levels of oxygen consumption correlates with the levels of the target analyte present. Thus, bilirubin and ascorbic acid levels can be measured indirectly by electrochemically sensing oxygen level changes, as in a Clark type electrode setup, for example.

Alternatively, a different configuration for sensing bilirubin and ascorbic acid can be employed. For example, an electrode domain including one or more electrode domains comprising electron transfer agents, such as NAFION™, polyzwitterion polymers, or polymeric mediator can be coated on the electrode. Measured oxygen levels transduced from such enzyme domain configurations can be correlated with the concentrations of bilirubin and ascorbic acid levels. In one example, an electrode domain comprising one or more mediators electrically coupled to a working electrode can be employed and correlated to the levels of bilirubin and ascorbic acid levels.

In one example, the aforementioned continuous bilirubin and ascorbic acid sensor configurations can be combined with any one of the aforementioned continuous alcohol sensor configurations, continuous uric acid sensor configurations, continuous cholesterol sensor configurations to provide a continuous multi-analyte sensor device as further described below. This continuous multi-analyte sensor device can further include continuous glucose monitoring capability. Other membranes can be used in the aforementioned continuous bilirubin and ascorbic acid sensor configuration, such as electrode, resistance, bio-interfacing, and drug releasing membranes.

### One-Working-Electrode Configurations for Dual Analyte Detection

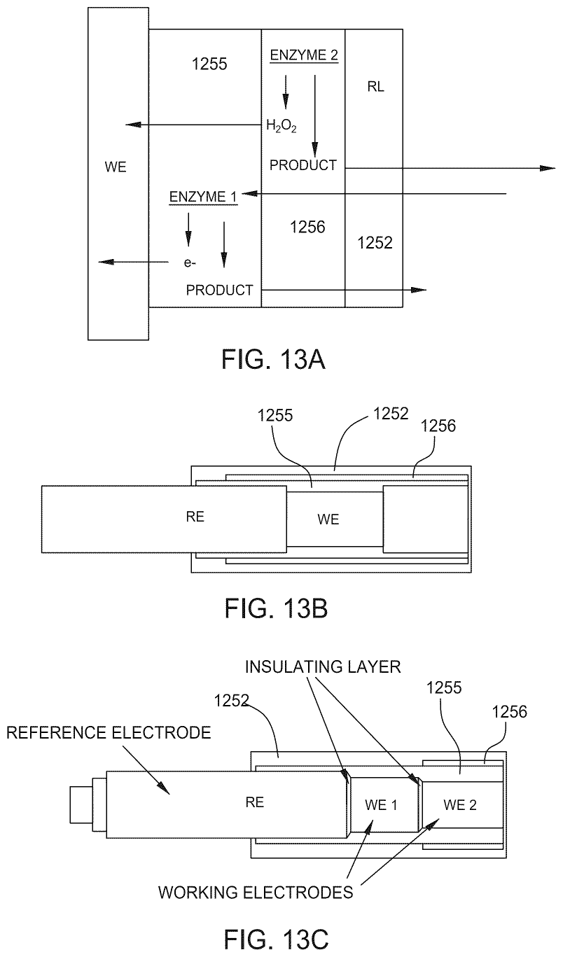

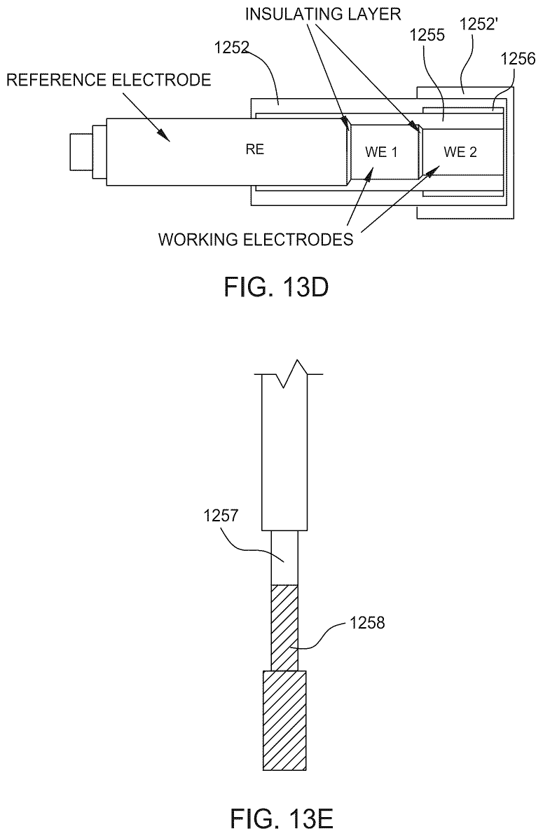

In one example, at least a dual enzyme domain configuration in which each layer contains one or more specific enzymes and optionally one or more cofactors is provided. In a broad sense, one example of a continuous multi-analyte sensor configuration is depicted in FIG. 13A where a first membrane 1255 (EZL1) comprising at least one enzyme (Enzyme 1) of the at least two enzyme domain configuration is proximal to at least one surface of a WE. One or more analyte-substrate enzyme pairs with Enzyme 1 transduces at least one detectable signal to the WE surface by direct electron transfer or by mediated electron transfer that corresponds directly or indirectly to an analyte concentration. Second membrane 1256 (EZL2) with at least one second enzyme (Enzyme 2) is positioned adjacent 1255 ELZ1, and is generally more distal from WE than EZL1. One or more resistance domains (RL) 1252 can be provided adjacent EZL2 1256, and/or between EZL1 1255 and EZL2 1256. The different enzymes catalyze the transformation of the same analyte, but at least one enzyme in EZL2 1256 provides hydrogen peroxide and the other at least one enzyme in EZL1 1255 does not provide hydrogen peroxide. Accordingly, each measurable species (e.g., hydrogen peroxide and the other measurable species that is not hydrogen peroxide) generates a signal associated with its concentration.

For example, in the configuration shown in FIG. 13A, a first analyte diffuses through RL 1252 and into EZL2 1256 resulting in peroxide via interaction with Enzyme 2. Peroxide diffuses at least through EZL1 1255 to WE and transduces a signal that corresponds directly or indirectly to the first analyte concentration. A second analyte, which is different from the first analyte, diffuses through RL 1252 and EZL2 1256 and interacts with Enzyme 1, which results in electron transfer to WE and transduces a signal that corresponds directly or indirectly to the second analyte concentration.

As shown in FIG. 13B, the above configuration is adapted to a conductive wire electrode construct, where at least two different enzyme-containing layers are constructed on the same WE with a single active surface. In one example, the single WE is a wire, with the active surface positioned about the longitudinal axis of the wire. In another example, the single WE is a conductive trace on a substrate, with the active surface positioned about the longitudinal axis of the trace. In one example, the active surface is substantially continuous about a longitudinal axis or a radius.

In the configuration described above, at least two different enzymes can be used and catalyze the transformation of different analytes, with at least one enzyme in EZL2 1256 providing hydrogen peroxide and the at least other enzyme in EZL1 1255 not providing hydrogen peroxide, e.g., providing electron transfer to the WE surface corresponding directly or indirectly to a concentration of the analyte.

In one example, an inner layer of the at least two enzyme domains EZL1, EZL2 1255, 1256 comprises at least one immobilized enzyme in combination with at least one mediator that can facilitate lower bias voltage operation of the WE than without the mediator. In one example, for such direct electron transductions, a potential P1 is used. In one example, at least a portion of the inner layer EZL1 1255 is more proximal to the WE surface and can have one or more intervening electrode domains and/or overlaying interference and/or bio-interfacing and/or drug releasing membranes, provided that the at least one mediator can facilitate low bias voltage operation with the WE surface. In another example, at least a portion of the inner layer EZL1 1255 is directly adjacent the WE.

The second layer of at least dual enzyme domain (the outer layer EZL2 1256) of FIG. 13B contains at least one enzyme that result in one or more catalysis reactions that eventually generate an amount of hydrogen peroxide that can electrochemically transduce a signal corresponding to the concentration of the analyte(s). In one example, the generated hydrogen peroxide diffuses through layer EZL2 1256 and through the inner layer EZL1 1255 to reach the WE surface and undergoes redox at a potential of P2, where P2 P1. In this way electron transfer and electrolysis (redox) can be selectively controlled by controlling the potentials P1, P2 applied at the same WE surface. Any applied potential durations can be used for P1, P2, for example, equal/periodic durations, staggered durations, random durations, as well as various potentiometric sequences, cyclic voltammetry etc. In some examples, impedimetric sensing can be used. In one example, a phase shift (e.g., a time lag) can result from detecting two signals from two different working electrodes, each signal being generated by a different EZL (EZL1, EZL2, 1255, 1256) associated with each electrode. The two (or more) signals can be broken down into components to detect the individual signal and signal artifacts generated by each of EZL1 1255 and EZL2 1256 in response to the detection of two analytes. In some examples, each EZL detects a different analyte. In other examples, both EZLs detect the same analyte.

In another alternative exemplary configuration, as shown in FIGS. 13C-13D a multienzyme domain configuration as described above is provided for a continuous multi-analyte sensor device using a single WE with two or more active surfaces is provided. In one example, the multienzyme domain configurations discussed herein are formed on a planar substrate. In another example, the single WE is coaxial, e.g., configured as a wire, having two or more active surfaces positioned about the longitudinal axis of the wire. Additional wires can be used, for example, as a reference and/or counter electrode. In another example, the single WE is a conductive trace on a substrate, with two or more active surfaces positioned about the longitudinal axis of the trace. At least a portion of the two or more active surfaces are discontinuous, providing for at least two physically separated WE surfaces on the same WE wire or trace. (e.g., WE1, WE2). In one example, the first analyte detected by WE1 is glucose, and the second analyte detected by WE2 is lactate. In another example, the first analyte detected by WE1 is glucose, and the second analyte detected by WE2 is ketones.

Thus, FIGS. 13C-13D depict exemplary configurations of a continuous multi-analyte sensor construct in which EZL1 1255, EZL2 1256 and RL 1252 (resistance domain) as described above, arranged, for example, by sequential dip coating techniques, over a single coaxial wire comprising spatially separated electrode surfaces WE1, WE2. One or more parameters, independently, of the enzyme domains, resistance domains, etc., can be controlled along the longitudinal axis of the WE, for example, thickness, length along the axis from the distal end of the wire, etc. In one example, at least a portion of the spatially separated electrode surfaces are of the same composition. In another example, at least a portion of the spatially separated electrode surfaces are of different composition. In FIGS. 13C-13D, WE1 represents a first working electrode surface configured to operate at P1, for example, and is electrically insulated from second working electrode surface WE2 that is configured to operate at P2, and RE represents a reference electrode RE electrically isolated from both WE1, WE2. One resistance domain is provided in the configuration of FIG. 13C that covers the reference electrode and WE1, WE2. An additional resistance domain is provided in the configuration of FIG. 13D that covers extends over essentially WE2 only. Additional electrodes, such as a counter electrode can be used. Such configurations (whether single wire or dual wire configurations) can also be used to measure the same analyte using two different techniques. Using different signal generating sequences as well as different RLs, the data collected from two different mode of measurements provides increase fidelity, improved performance and device longevity. A non-limiting example is a glucose oxidase (H2O2 producing) and glucose dehydrogenase (electrically coupled) configuration. Measurement of Glucose at two potentials and from two different electrodes provides more data points and accuracy. Such approaches may not be needed for glucose sensing, but the can be applied across the biomarker sensing spectrum of other analytes, alone or in combination with glucoses sensing, such as ketone sensing, ketone/lactate sensing, and ketone/glucose sensing.

In an alternative configuration of that depicted in FIGS. 13C-13D, two or more wire electrodes, which can be colinear, wrapped, or otherwise juxtaposed, are presented, where WE1 is separated from WE2, for example, from other elongated shaped electrode. Insulating layer electrically isolates WE1 from WE2. In this configuration, independent electrode potential can be applied to the corresponding electrode surfaces, where the independent electrode potential can be provided simultaneously, sequentially, or randomly to WE1, WE2. In one example, electrode potentials presented to the corresponding electrode surfaces WES1, WES2, are different. One or more additional electrodes can be present such as a reference electrode and/or a counter electrode. In one example, WES2 is positioned longitudinally distal from WES1 in an elongated arrangement. Using, for example, dip coating methods, WES1 and WES2 are coated with enzyme domain EZL1, while WES2 is coated with different enzyme domain EZL2. Based on the dipping parameters, or different thickness of enzyme domains, multi-layered enzyme domains, each layer independently comprising different loads and/or compositions of enzyme and/or cofactors, mediators can be employed. Likewise, one or more resistance domains (RL) can be applied, each can be of a different thickness along the longitudinal axis of the electrode, and over different electrodes and enzyme domains by controlling dip length and other parameters, for example. With reference to FIG. 13D, such an arrangement of RL's is depicted, where an additional RL 1252′ is adjacent WES2 but substantially absent from WES1.

In one example of measuring two different analytes, the above configuration comprising enzyme domain EZL1 1255 comprising one or more enzyme(s) and one or more mediators for at least one enzyme of EZL1 to provide for direct electron transfer to the WES1 and determining a concentration of at least a first analyte. In addition, enzyme domain EZL2 1256 can comprise at least one enzyme that provides peroxide (e.g., hydrogen peroxide) or consumes oxygen during catalysis with its substrate. The peroxide or the oxygen produced in EZL2 1256 migrates to WES2 and provides a detectable signal that corresponds directly or indirectly to a second analyte. For example, WES2 can be carbon, wired to glucose dehydrogenase to measure glucose, while WES1 can be platinum, that measures peroxided produced from lactate oxidase/lactate in EZL2 1256. The combinations of electrode material and enzyme(s) as disclosed herein are examples and non-limiting.

In one example, the potentials of P1 and P2 can be separated by an amount of potential so that both signals (from direct electron transfer from EZL1 1255 and from hydrogen peroxide redox at WE) can be separately activated and measured. In one example, the electronic module of the sensor can switch between two sensing potentials continuously in a continuous or semi-continuous periodic manner, for example a period (t1) at potential P1, and period (t2) at potential P2 with optionally a rest time with no applied potential. Signal extracted can then be analyzed to measure the concentration of the two different analytes. In another example, the electronic module of the sensor can undergo cyclic voltammetry, providing changes in current when swiping over potentials of P1 and P2 can be correlated to transduced signal coming from either direct electron transfer or electrolysis of hydrogen peroxide, respectably. In one example, the modality of sensing is non-limiting and can include different amperometry techniques, e.g., cyclic voltammetry. In one example, an alternative configuration is provided but hydrogen peroxide production in EZL2 is replaced by another suitable electrolysis compound that maintains the P2 ¥ P1 relationship, such as oxygen, and at least one enzyme-substrate combination that provide the other electrolysis compound.

For example, a continuous multi-analyte sensor configuration, for choline and glucose, in which enzyme domains EZ1 1255, EZ2 1256 were associated with different WEs, e.g., platinum WE2, and gold WE1 was prepared. In this exemplary case, EZL1 1255 contained glucose oxidase and a mediator coupled to WE1 to facilitate electron direct transfer upon catalysis of glucose, and EZL2 1256 contained choline oxidase that will catalyze choline and generate hydrogen peroxide for electrolysis at WE2. The EZL's were coated with resistance domains; upon cure and readiness they underwent cyclic voltammetry in the presence of glucose and choline. A wired glucose oxidase enzyme to a gold electrode is capable of transducing signal at 0.2 volts, therefore, by analyzing the current changes at 0.2 volts, the concentration of glucose can be determined. The data also demonstrates that choline concentration is also inferentially detectable at the WE2 platinum electrode if the CV trace is analyzed at the voltage P2.

In one example, either electrode WE1 or WE2 can be, for example, a composite material, for example a gold electrode with platinum ink deposited on top, a carbon/platinum mix, and or traces of carbon on top of platinum, or porous carbon coating on a platinum surface. In one example, with the electrode surfaces containing two distinct materials, for example, carbon used for the wired enzyme and electron transfer, while platinum can be used for hydrogen peroxide redox and detection. As shown in FIG. 13E, an example of such composite electrode surfaces is shown, in which an extended platinum covered wire 1257 is half coated with carbon 1258, to facilitate multi sensing on two different surfaces of the same electrode. In one example WE2 can be grown on or extend from a portion of the surface or distal end of WE1, for example, by vapor deposition, sputtering, or electrolytic deposition and the like.

Additional examples include a composite electrode material that can be used to form one or both of WE1 and WE2. In one example, a platinum-carbon electrode WE1, comprising EZL1 with glucose dehydrogenase is wired to the carbon surface, and outer EZL2 comprising lactate oxidase generating hydrogen peroxide that is detectable by the platinum surface of the same WE1 electrode. Other examples of this configuration can include ketone sensing (beta-hydroxybutyrate dehydrogenase electrically coupled enzyme in EZL1 1255) and glucose sensing (glucose oxidase in EZL2 1256). Other membranes can be used in the aforementioned configuration, such as electrode, resistance, bio-interfacing, and drug releasing membranes. In other examples, one or both of the working electrodes (WE1, WE2) can be gold-carbon (Au—C), palladium-carbon (Pd—C), iridium-carbon (Ir—C), rhodium-carbon (Rh—C), or ruthenium-carbon (Ru—C). In some examples, the carbon in the working electrodes discussed herein can instead or additionally include graphene, graphene oxide, or other materials suitable for forming the working electrodes, such as commercially available carbon ink.

### Glycerol Sensor Configurations

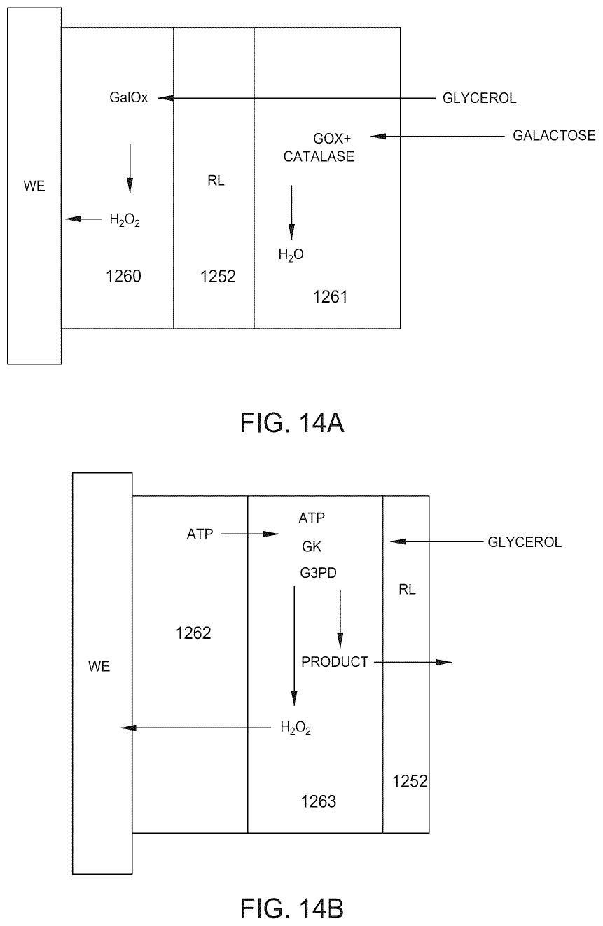

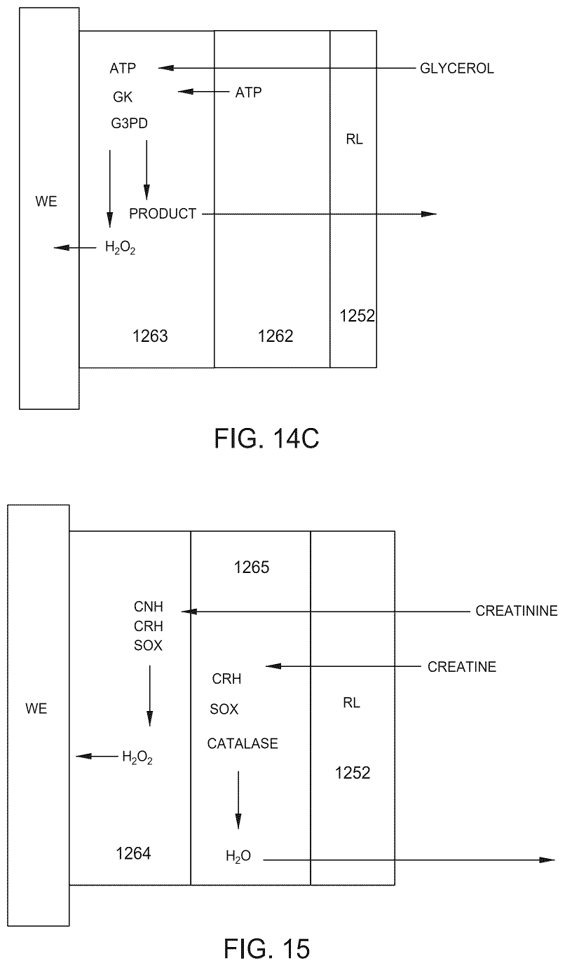

As shown in FIG. 14A, an exemplary continuous glycerol sensor configuration is depicted where a first enzyme domain EZL1 1260 comprising galactose oxidase is positioned proximal to at least a portion of a WE surface. A second enzyme domain EZL2 1261 comprising glucose oxidase and catalase is positioned more distal from the WE. As shown in FIG. 14A, one or more resistance domains (RL) 1252 are positioned between EZL1 1260 and EZL2 1261. Additional RLs can be employed, for example, adjacent to EZL2 1261. Modification of the one or more RL membranes to attenuate the flux of either analyte and increase glycerol to galactose sensitivity ratio is envisaged. The above glycerol sensing configuration provides for a glycerol sensor that can be combined with one or more additional sensor configurations as disclosed herein.

Glycerol can be catalyzed by the enzyme galactose oxidase (GalOx), however, GalOx has an activity ratio of 1%-5% towards glycerol. In one example, the activity of GalOx towards this secondary analyte glycerol can be utilized. The relative concentrations of glycerol in vivo are much higher that galactose (˜2 μmol/l for galactose, and ˜100 μmol/l for glycerol), which compliments the aforementioned configurations.

If the GalOx present in EZL1 1260 membrane is not otherwise functionally limited, then the GalOx will catalyze most if not all of the glycerol that passes through the one or more RLs. The signal contribution from the glycerol present will be higher as compared to the signal contribution from galactose. In one example, the one or more RL's are chemically configured to provide a higher influx of glycerol or a lower influx of galactose.

In another example, a glycol sensor configuration is provided using multiple working electrodes WEs that provides for utilizing signal transduced from both WEs. Utilizing signal transduced from both WEs can provide increasing selectivity. In one example EZL1 1260 and EZL2 1261 comprise the same oxidase enzyme (e.g., galactose oxidase) with different ratios of enzyme loading, and/or a different immobilizing polymer and/or different number and layers of RL's over the WEs. Such configurations provide for measurement of the same target analyte with different sensitivities, resulting in a dual measurement. Using a mathematical algorithm to correct for noise and interference from a first signal, and inputting the first signal from one sensing electrode with a first analyte sensitivity ratio into the mathematical algorithm, allows for the decoupling of the second signal corresponding to the desired analyte contributions. Modification of the sensitivity ratio of the one or more EZL's to distinguish signals from the interfering species and the analyte(s) of interest can be provided by adjusting one or more of enzyme source, enzyme load in EZL's, chemical nature/diffusional characteristics of EZL's, chemical/diffusional characteristics of the at least one RL's, and combinations thereof.

As discussed herein, a secondary enzyme domain can be utilized to catalyze the non-target analyte(s), reducing their concentration and limiting diffusion towards the sensing electrode through adjacent membranes that contains the primary enzyme and necessary additives. In this example, the most distal enzyme domain, EZL2, 1261 is configured to catalyze a non-target analyte that would otherwise react with EZL1, thus providing a potentially less accurate reading of the target analyte (glycerol) concentration. This secondary enzyme domain can act as a “selective diffusion exclusion membrane” by itself, or in some other configurations can be placed above or under a resistant layer (RL) 1252. In this example, the target analyte is glycerol and GalOX is used to catalyze glycerol to form a measurable species (e.g., hydrogen peroxide).

In one example, a continuous glycerol sensor configuration is provided using at least glycerol oxidase, which provides hydrogen peroxide upon reaction and catalysis of glycerol. Thus, in one example, enzyme domain comprising glycerol oxidase can be positioned adjacent at least a portion of a WE surface and hydrogen peroxide is detected using amperometry. In another example, enzyme domain comprising glycerol oxidase is used for sensing oxygen level changes, for example, in a Clark type electrode setup. Alternatively, at least a portion of the WE surface can be coated with one more layers of electrically coupled polymers, such as a mediator system discussed below, to provide a coated WE capable of electron transfer from the enzyme at a lower potential. The coated WE can then operate at a different and lower voltage to measure oxygen and its correlation to glycerol concentration.

In another example, a glycerol sensor configuration is provided using glycerol-3-phosphate oxidase in the enzyme domain. In one example, ATP is used as the cofactor. Thus, as shown in FIGS. 14B and 14C, exemplary sensor configurations are depicted where in one example (FIG. 14B), one or more cofactors 962 (e.g., ATP) is proximal to at least a portion of an WE surface. One or more enzyme domains 1263 comprising glycerol-3-phospohate oxidase (G3PD), lipase, and/or glycerol kinase (GK) and one or more regenerating enzymes capable of continuously regenerating the cofactor are contained in an enzyme domain are adjacent the cofactor, or more distal from the WE surface than the cofactor layer 962. Examples of regenerating enzymes that can be used to provide ATP regeneration include, but are not limited to, ATP synthase, pyruvate kinase, acetate kinase, and creatine kinase. The one or more regenerating enzymes can be included in one or more enzyme domains, or in a separate layer.

An alternative configuration is shown in FIG. 14C, where one or more enzyme domains 1263 comprising G3PD, at least one cofactor and at least one regenerating enzyme, are positioned proximal to at least a portion of WE surface, with one or more cofactor reservoirs 962 adjacent to the enzyme domains comprising G3PD and more distal from the WE surface, and one or more RL's 1252 are positioned adjacent the cofactor reservoir. In either of these configurations, an additional enzyme domain comprising lipase can be included to indirectly measure triglyceride, as the lipase will produce glycerol for detection by the aforementioned glycerol sensor configurations.

In another example, a glycerol sensor configuration is provided using dehydrogenase enzymes with cofactors and regenerating enzymes. In one example, cofactors that can be incorporated in the one or more enzyme domains include one or more of NAD(P)H, NADP+, and ATP. In one example, e.g., for use of NAD(P)H a regenerating enzyme can be NADH oxidase or diaphorase to convert NADH, the product of the dehydrogenase catalysis back to NAD(P)H. Similar methodologies can be used for creating other glycerol sensors, for example, glycerol dehydrogenase, combined with NADH oxidase or diaphorase can be configured to measure glycerol or oxygen.

In one example, mathematical modeling can be used to identify and remove interference signals, measuring very low analyte concentrations, signal error and noise reduction so as to improve and increase of multi-analyte sensor end of life. For example, with a two WE electrode configuration where WE1 is coated with a first EZL while WE2 is coated with two or more different EZL, optionally with one or more resistance domains (RL) a mathematical correction such interference can be corrected for, providing for increasing accuracy of the measurements.

Changes of enzyme load, immobilizing polymer and resistance domain characteristics over each analyte sensing region can result in different sensitive ratios between two or more target analyte and interfering species. If the signal are collected and analyzed using mathematical modeling, a more precise concentration of the target analytes can be calculated.

One example in which use of mathematical modeling can be helpful is with glycerol sensing, where galactose oxidase is sensitive towards both galactose and glycerol. The sensitivity ratio of galactose oxidase to glycerol is about is 1%-5% of its sensitivity to galactose. In such case, modification of the sensitivity ratio to the two analytes is possible by adjusting the one or more parameters, such as enzyme source, enzyme load, enzyme domain (EZL) diffusional characteristics, RL diffusional characteristics, and combinations thereof. If two WEs are operating in the sensor system, signal correction and analysis from both WEs using mathematical modeling provides high degree of fidelity and target analyte concentration measurement.

In the above configurations, the proximity to the WE of one or more of these enzyme immobilizing layers discussed herein can be different or reversed, for example if the most proximal to the WE enzyme domain provides hydrogen peroxide, this configuration can be used.

In some examples, the target analyte can be measured using one or multiple of enzyme working in concert. In one example, ATP can be immobilized in one or more EZL membranes, or can be added to an adjacent layer alone or in combination with a secondary cofactor, or can get regenerated/recycled for use in the same EZL or an adjacent third EZL. This configuration can further include a cofactor regenerator enzyme, e.g., alcohol dehydrogenase or NADH oxidase to regenerate NAD(P)H. Other examples of cofactor regenerator enzymes that can be used for ATP regeneration are ATP synthase, pyruvate kinase, acetate kinase, creatine kinase, and the like.

In one example, the aforementioned continuous glycerol sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations to provide a continuous multi-analyte sensor device as further described below. This continuous multi-analyte sensor device can further include continuous glucose monitoring capability. Other configurations can be used in the aforementioned continuous glycerol sensor configuration, such as electrode, resistance, bio-interfacing, and drug releasing membranes.

### Creatinine Sensor Configurations

In one example, continuous creatinine sensor configurations are provided, such configurations containing one or more enzymes and/or cofactors. Creatinine sensor configurations are examples of continuous analyte sensing systems that generate intermediate, interfering products, where these intermediates/interferents are also present in the biological fluids sampled. The present disclosure provides solutions to address these technical problems and provide for accurate, stable, and continuous creatinine monitoring alone or in combination with other continuous multi-analyte sensor configurations.

Creatinine sensors, when in use, are subject to changes of a number of physiologically present intermediate/interfering products, for example sarcosine and creatine, that can affect the correlation of the transduced signal with the creatinine concentration. The physiological concentration range of sarcosine, for example, is an order of magnitude lower that creatinine or creatine, so signal contribution from circulating sarcosine is typically minimal. However, changes in local physiological creatine concentration can affect the creatinine sensor signal. In one example, eliminating or reducing such signal contribution is provided.

Thus, in one example, eliminating or reducing creatine signal contribution of a creatinine sensor comprises using at least one enzyme that will consume the non-targeted interfering analyte, in this case, creatine. For example, two enzyme domains are used, positioned adjacent to each other. At least a portion of a first enzyme domain is positioned proximal to at least a portion of a WE surface, the first enzyme domain comprising one or more enzymes selected from creatinine amidohydrolase (CNH), creatine amidohydrolase (CRH), and sarcosine oxidase (SOX). A second enzyme domain, adjacent the first enzyme domain and more distal from the WE surface, comprises one or more enzymes using creatine as their substrate so as to eliminate or reduce creatine diffusion towards the WE. In one example, combinations of enzymes include CRH, SOX, creatine kinase, and catalase, where the enzyme ratios are tuned to provide ample number of units such that circulating creatine will at least partially be consumed by CRH providing sarcosine and urea, whereas the sarcosine produced will at least partially be consumed by SOX, providing an oxidized form of glycine (e.g. glycine aldehyde) which will at least be partially consumed by catalase. In an alternative configuration of the above, the urea produced by the CRH catalysis can at least partially be consumed by urease to provide ammonia, with the aqueous form (NH4+) being detected via an ion-selective electrode (e.g., nonactin ionophore). Such an alternative potentiometric sensing configuration can provide an alternative to amperometric peroxide detection (e.g., improved sensitivity, limits of detection, and lack of depletion of the reference electrode, alternate pathways/mechanisms). This dual-analyte-sensing example can include a creatinine-potassium sensor having potentiometric sensing at two different working electrodes. In this example, interference signals can be identified and corrected. In one alternative example, the aforementioned configuration can include multi-modal sensing architectures using a combination of amperometry and potentiometry to detect concentrations of peroxide and ammonium ion, measured using amperometry and potentiometry, respectively, and correlated to measure the concentration of the creatinine. In one example, the aforementioned configurations can further comprise one or more configurations (e.g., without enzyme) separating the two enzyme domains to provide complementary or assisting diffusional separations and barriers.

In yet another example, a method to isolate the signal and measure essentially only creatinine is to use a second WE that measures the interfering species (e.g., creatine) and then correct for the signal using mathematical modeling. Thus, for example, signal from the WE interacting with creatine is used as a reference signal. Signal from another WE interacting with creatinine is from corrected for signal from the WE interacting with creatine to selectively determine creatinine concentration.

In yet another example, sensing creatinine is provided by measuring oxygen level changes electrochemically, for example in a Clark type electrode setup, or using one or more electrodes coated with layers of different polymers such as NAFION™ and correlating changes of potential based on oxygen changes, which will indirectly correlate with the concentrations of creatinine.

In yet another example, sensing creatinine is provided by using sarcosine oxidase wired to at least one WE using one or more electrically coupled mediators. In this approach, concentration of creatinine will indirectly correlate with the electron transfer generated signal collected from the WE.

For the aforementioned creatinine sensor configurations based on hydrogen peroxide and/or oxygen measurements the one or more enzymes can be in a single enzyme domain, or the one or more enzymes, independently, can be in one or more enzyme domains, or any other combination thereof, in which in each layer at least one enzyme is present. For the aforementioned creatinine sensor configurations based on use of an electrically coupled sarcosine oxidase containing layer, the layer positioned adjacent to the electrode and is electrically coupled to at least a portion of the electrode surface using mediators.

In another example, the aforementioned creatinine sensor configurations can be sensed using potentiometry by using urease enzyme (UR) that creates ammonium from urea, the urea created by CRH from creatine, the creatine being formed from the interaction of creatinine with CNH. Thus, ammonium can be measured by the above configuration and correlated with the creatinine concentration. Alternatively, creatine amidohydrolase (CI) or creatinine deiminase can be used to create ammonia gas, which under physiological conditions of a transcutaneous sensor, would provide ammonium ion for signal transduction.

In yet another example, sensing creatinine is provided by using one or more enzymes and one or more cofactors. Some non-limiting examples of such configurations include creatinine deaminase (CD) providing ammonium from creatinine, glutamate dehydrogenase (GLDH) providing peroxide from the ammonium, where hydrogen peroxide correlates with levels of present creatinine. The above configuration can further include a third enzyme glutamate oxidase (GLOD) to further break down glutamate formed from the GDLH and create additional hydrogen peroxide. Such combinations of enzymes, independently, can be in one or more enzyme domains, or any other combination thereof, in which in each domain or layer, at least one enzyme is present.

In yet another example, sensing creatinine is provided by the combination of creatinine amidohydrolase (CNH), creatine kinase (CK) and pyruvate kinase (PK), where pyruvate, created by PK can be detected by one or more of either lactate dehydrogenase (LDH) or pyruvate oxidase (POX) enzymes configured independently, where one or more of the aforementioned enzyme are present in one layer, or, in which in each of a plurality of layers comprises at least one enzyme, any other combination thereof.

In such sensor configurations where one or more cofactors and/or regenerating enzymes for the cofactors are used, providing excess amounts of one or more of NADH, NAD(P)H and ATP in any of the one or more configurations can be employed, and one or more diffusion resistance domains can be introduced to limit or prevent flux of the cofactors from their respective membrane(s). Other configurations can be used in the aforementioned configurations, such as electrode, resistance, bio-interfacing, and drug releasing membranes.

In yet another example, creatinine detection is provided by using creatinine deiminase in one or more enzyme domains and providing ammonium to the enzyme domain(s) via catalysis of creatinine. Ammonium ion can then be detected potentiometrically or by using composite electrodes that undergo redox when exposed to ammonium ion, for example NAFION™/polyaniline composite electrodes, in which polyaniline undergoes redox in the presence of ammonium at the electrode under potential. Ammonium concentration can then be correlated to creatinine concentration.

FIG. 15 depicts an exemplary continuous sensor configuration for creatinine. In the example of FIG. 15, the sensor includes a first enzyme domain 1264 comprising CNH, CRH, and SOX are adjacent a working electrode WE, e.g., platinum. A second enzyme domain 1265 is positioned adjacent the first enzyme domain and is more distal from the WE. One or more resistance domains (RL) 1252 can be positioned adjacent the second enzyme domain or between the first and second layers. Creatinine is diffusible through the RL and the second enzyme domain to the first enzyme domain where it is converted to peroxide and transduces a signal corresponding to its concentration. Creatine is diffusible through the RL and is converted in the second enzyme domain to sarcosine and urea, the sarcosine being consumed by the sarcosine oxidase and the peroxide generated is consumed by the catalase, thus preventing transduction of the creatine signal.

For example, variations of the above configuration are possible for continuous monitoring of creatinine alone or in combination with one or more other analytes. Thus, one alternative approach to sensing creatinine could be sensing oxygen level changes electrochemically, for example in a Clark-type electrode setup. In one example, the WE can be coated with layers of different polymers, such as NAFION™ and based on changes of potential oxygen changes, the concentrations of creatinine can be correlated. In yet another example, one or more enzyme most proximal to the WE, i.e., sarcosine oxidase, can be “wired” to the electrode using one or more mediators. Each of the different enzymes in the above configurations can be distributed inside a polymer matrix or domain to provide one enzyme domain. In another example, one or more of the different enzymes discussed herein can be formed as the enzyme domain and can be formed layer by layer, in which each layer has at least one enzyme present. In an example of a “wired” enzyme configuration with a multilayered membrane, the wired enzyme domain would be most proximal to the electrode. One or more interferent layers can be deposited among the multilayer enzyme configuration so as to block of non-targeted analytes from reaching electrodes.

In one example, the aforementioned continuous creatinine sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations to provide a continuous multi-analyte sensor device as further described below. This continuous multi-analyte sensor device can further include continuous glucose monitoring capability.

### Lactose Sensor Configurations

In one example, a continuous lactose sensor configuration, alone or in combination with another analyte sensing configuration comprising one or more enzymes and/or cofactors is provided. In a general sense, a lactose sensing configuration using at least one enzyme domain comprising lactase enzyme is used for producing glucose and galactose from the lactose. The produced glucose or galactose is then enzymatically converted to a peroxide for signal transduction at an electrode. Thus, in one example, at least one enzyme domain EZL1 comprising lactase is positioned proximal to at least a portion of a WE surface capable of electrolysis of hydrogen peroxide. In one example, glucose oxidase enzyme (GOX) is included in EZL1, with one or more cofactors or electrically coupled mediators. In another example, galactose oxidase enzyme (GalOx) is included in EZL1, optionally with one or more cofactors or mediators. In one example, glucose oxidase enzyme and galactose oxidase are both included in EZL1. In one example, glucose oxidase enzyme and galactose oxidase are both included in EZL1, optionally with one or more cofactors or electrically coupled mediators.

One or more additional EZL's (e.g. EZL2) can be positioned adjacent the EZL1, where at least a portion of EZL2 is more distal from at least a portion of WE than EZL1. In one example, one or more layers can be positioned in between EZL1 and EZL2, such layers can comprise enzyme, cofactor or mediator or be essentially devoid of one or more of enzymes, cofactors or mediators. In one example, the one or more layers positioned in between EZL1 and EZL2 is essentially devoid of enzyme, e.g., no purposefully added enzyme. In one example one or layers can be positioned adjacent EZL2, being more distal from at least a portion of EZL1 than EZL2, and comprise one or more of the enzymes present in either EZL1 or EZL2.

In one example of the aforementioned lactose sensor configurations, the peroxide generating enzyme can be electrically coupled to the electrode using coupling mediators. The transduced peroxide signals from the aforementioned lactose sensor configurations can be correlated with the level of lactose present.

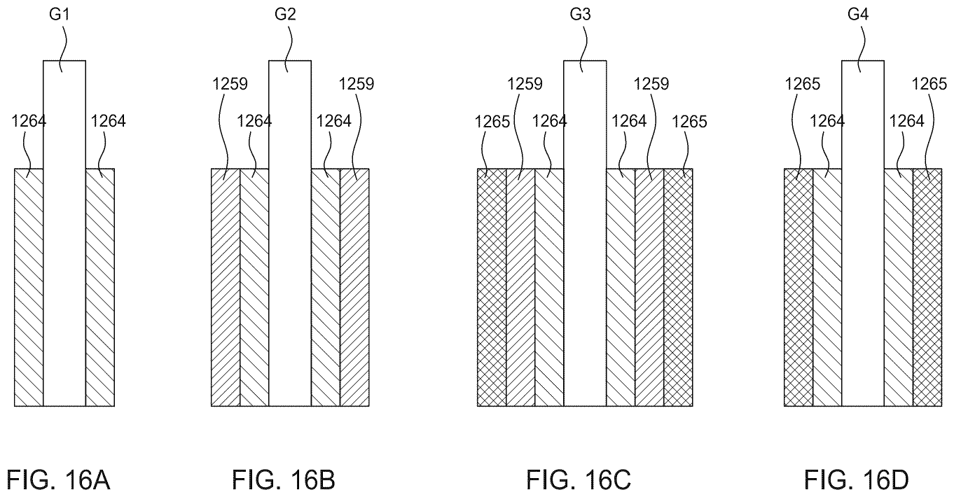

FIG. 16A-16D depict alternative continuous lactose sensor configurations. Thus, in an enzyme domain EZL1 1264 most proximal to WE (G1), comprising GalOx and lactase, provides a lactose sensor that is sensitive to galactose and lactose concentration changes and is essentially non-transducing of glucose concentration. As shown in FIGS. 16B-16D, additional layers, including non-enzyme containing layers 1259, and a lactase enzyme containing layer 1265, and optionally, electrode, resistance, bio-interfacing, and drug releasing membranes. (not shown) are used. Since changes in physiological galactose concentration are minimal, the transduced signal would essentially be from physiological lactose fluctuations.

In one example, the aforementioned continuous lactose sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations, creatinine sensor configurations to provide a continuous multi-analyte sensor device as further described below. This continuous multi-analyte sensor device can further include continuous glucose monitoring capability. Other membranes can be used in the aforementioned sensor configuration, such as electrode, resistance, bio-interfacing, and drug releasing membranes.

### Urea Sensor Configurations

Similar approach as described above can also be used to create a continuous urea sensor. For example urease (UR), which can break down the urea and to provide ammonium can be used in an enzyme domain configuration. Ammonium can be detected with potentiometry or by using a composite electrodes, e.g., electrodes that undergo redox when exposed to ammonium. Example electrodes for ammonium signal transduction include, but are not limited to, NAFION™/polyaniline composite electrodes, in which polyaniline undergoes redox in the presence of ammonium at an applied potential, with essentially direct correlation of signal to the level of ammonium present in the surrounding. This method can also be used to measure other analytes such as glutamate using the enzyme glutaminase (GLUS).

In one example, the aforementioned continuous uric acid sensor configurations can be combined with any one of the aforementioned continuous alcohol sensor configurations and/or continuous uric acid sensor configurations and/or continuous cholesterol sensor configurations and/or continuous bilirubin/ascorbic acid sensor configurations and/or continuous ketone sensor configurations and/or continuous choline sensor configurations and/or continuous glycerol sensor configurations and/or continuous creatinine sensor configurations and/or continuous lactose sensor configurations to provide a continuous multi-analyte sensor device as further described below. This continuous multi-analyte sensor device can further include continuous glucose monitoring capability. Other membranes can be used in the aforementioned uric acid sensor configuration, such as electrode, resistance, bio-interfacing, and drug releasing membranes.

### ADDITIONAL CONSIDERATIONS

The methods disclosed herein comprise one or more steps or actions for achieving the methods. To the extent the results of the method are still realized, method steps and/or actions can be performed in different order without departing from the scope of the claims. In other words, unless a specific order of steps or actions is specified, the order and/or use of specific steps and/or actions can be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein can be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including.” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications can be practiced. Therefore, the description and examples should not be construed as limiting the scope of the invention to the specific examples and examples described herein, but rather to also cover all modification and alternatives coming with the true scope and spirit of the invention.

1. A continuous analyte monitoring system, comprising:
one or more continuous analyte sensors configured to penetrate a skin of a user and generate one or more sensor signals indicative of one or more analyte levels of the user;
at least one sensor electronics module coupled to the one or more continuous analyte sensors, wherein the at least one sensor electronics module comprises:
an analog to digital converter configured to:
receive the sensor signal; and
convert the sensor current generated by the one or more continuous analyte sensors into digital signals;
a wireless transceiver configured to transmit the digital signals wirelessly to a wireless communications device using a standardized wireless transmission protocol; and
one or more memories comprising executable instructions; and
one or more processors in data communication with the one or more memories and configured to execute the executable instructions to:
convert the digital signals to a set of estimated analyte measurements indicative of true analyte levels of the user; and
generate therapy management guidance for the user based on the estimated analyte measurements.

2. The monitoring system of claim 1, wherein the sensor electronic module further comprises a sensitivity profile for the monitoring system based on a calibration process performed during manufacturing, wherein one or more processors being configured to convert the digital signals to the set of analyte measurements comprises converting the digital signals to the set of analyte measurements based on the sensitivity profile.

3. The monitoring system of claim 1, wherein continuous analyte sensor comprises:
a percutaneous wire comprising:
a proximal portion coupled to the sensor electronics module; and
a distal portion comprising a working electrode and a reference electrode, wherein the working electrode is configured to penetrate the skin and extend into a dermis or subcutaneous tissue of the user.

4. The monitoring system of claim 3, wherein:
the working electrode and the reference electrodes are disposed on a substrate, and
the sensor current is at least in part based on a voltage difference generated between the working electrode and the reference electrode.

5. The monitoring system of claim 1, wherein:
the continuous analyte sensor comprises a continuous potassium sensor;
the set of estimated analyte measurements comprises potassium measurements; and
the true analyte levels of the user comprise potassium levels of the user.

6. The monitoring system of claim 5, wherein the one or more processors are further configured to execute the executable instructions to:
determine, based on the set of estimated analyte measurements, one or more potassium metrics indicating a trend of the potassium levels of the user.

7. The monitoring system of claim 6, wherein the therapy management guidance is determined based on a first comparison of the one or more potassium metrics to one or more potassium thresholds of the user.

8. The monitoring system of claim 7, wherein:
the first comparison indicates that:
the potassium levels of the user are not stable; and
the potassium levels of the user are above a first range normal for the user; and
the therapy management guidance comprises guidance related to a risk of cardiac event or acute kidney failure.

9. The monitoring system of claim 7, wherein:
the continuous analyte sensor further comprises at least one of a continuous ammonia sensor or a continuous sodium sensor,
the set of analyte measurements comprises at least one of ammonia measurements or sodium measurements;
the true analyte levels of the user comprise at least one of ammonia levels or sodium levels of the user; and
the one or more processors are further configured to execute the executable instructions to:
determine, based on the set of estimated analyte measurements, at least one of an ammonia metric or a sodium metric indicating a trend of the ammonia levels or sodium levels of the user.

10. The monitoring system of claim 9, wherein the therapy management guidance is determined based on the first comparison and a second comparison of the at least one of the ammonia metric or the sodium metric to one or more ammonia thresholds or sodium thresholds of the user.

11. The monitoring system of claim 10, wherein:
the first comparison indicates that the potassium levels of the user are below a first range normal for the user;
the second comparison indicates that the at least one of the ammonia levels or the sodium levels of the user are above a second range normal for the user; and
the therapy management guidance comprises guidance related to a presence or risk of developing hepatic encephalopathy.

12. The monitoring system of claim 1, wherein:
the continuous analyte sensor comprises a continuous lactate sensor;
the set of estimated analyte measurements comprises lactate measurements; and
the true analyte levels of the user comprise lactate levels of the user.

13. The monitoring system of claim 12, wherein the one or more processors are further configured to execute the executable instructions to:
determine, based on the set of estimated analyte measurements, one or more lactate metrics indicating a trend of the lactate levels of the user.

14. The monitoring system of claim 13, wherein the therapy management guidance is determined based on a first comparison of the one or more lactate metrics to one or more lactate thresholds of the user.

15. The monitoring system of claim 14, wherein:
the first comparison indicates that:
the lactate levels of the user are increasing at a slow rate; and
the lactate levels of the user are above a first range normal for the user; and
the therapy management guidance comprises guidance related to a worsening liver function of the user.

16. The monitoring system of claim 14, wherein:
the first comparison indicates that:
the lactate levels of the user are increasing at a fast rate; and
the lactate levels of the user are above a first range normal for the user; and
the therapy management guidance comprises guidance related to a presence or risk of at least one of hepatic encephalopathy, sepsis, esophageal varices, a bleeding complication, or infection.

17. The monitoring system of claim 14, wherein:
the continuous analyte sensor further comprises a continuous ammonia sensor;
the set of analyte measurements comprises ammonia measurements;
the true analyte levels of the user comprise ammonia levels of the user; and
the one or more processors are further configured to execute the executable instructions to:
determine, based on the set of estimated analyte measurements, an ammonia metric indicating a trend of the ammonia levels of the user.

18. The monitoring system of claim 17, wherein the therapy management guidance is determined based on the first comparison and a second comparison of the ammonia metric to one or more ammonia thresholds of the user.

19. The monitoring system of claim 18, wherein:
the first comparison indicates that the lactate levels of the user are increasing;
the second comparison indicates that the ammonia levels of the user are increasing;
the increasing lactate levels precede the increasing ammonia levels; and
the therapy management guidance comprises guidance relating to a presence or risk of developing at least one of ascites, sepsis, or a bacterial positive infection.

20. The monitoring system of claim 18, wherein:
the first comparison indicates that the lactate levels of the user are increasing;
the second comparison indicates that the ammonia levels of the user are increasing;
the increasing lactate levels proceed the increasing ammonia levels; and
the therapy management guidance comprises guidance relating to a presence or risk of an acute health event.

21. The monitoring system of claim 20, wherein the acute health even comprises variceal bleeding.

22. The monitoring system of claim 1, wherein:
the continuous analyte sensor comprises a continuous ammonia sensor;
the set of estimated analyte measurements comprises ammonia measurements; and
the true analyte levels of the user comprise ammonia levels of the user.

23. The monitoring system of claim 22, wherein the one or more processors are further configured to execute the executable instructions to:
determine, based on the set of estimated analyte measurements, one or more ammonia metrics indicating a trend of the ammonia levels of the user.

24. The monitoring system of claim 23, wherein the therapy management guidance is determined based on the one or more ammonia metrics.

25. The monitoring system of claim 24, wherein:
the one or more processors are further configured to execute the executable instructions to:
receive electrolyte data and pH data associated with the user; and
determine, based on the electrolyte data and pH data, an electrolyte metric and a pH metric indicating trends of electrolyte levels and PH levels of the user; and
the therapy management guidance is determined based on the one or more ammonia metrics and a comparison of the electrolyte metric and the pH metric to one or more electrolyte thresholds and one or more pH thresholds, respectively, of the user.

26. The monitoring system of claim 25, wherein:
the one or more ammonia metrics indicate that the ammonia levels of the user are increasing at a slow rate;
the comparison indicates that the electrolyte levels and the pH levels of the user are outside of ranges normal for the user; and
the therapy management guidance comprises guidance relating to a risk of developing hepatic encephalopathy.

27. The monitoring system of claim 25, wherein:
the one or more ammonia metrics indicate that the ammonia levels of the user are increasing at a slow rate;
the comparison indicates that the electrolyte levels and the pH levels of the user are within ranges normal for the user; and
the therapy management guidance comprises guidance relating to a worsening liver function of the user.

28. The monitoring system of claim 24, wherein:
the continuous analyte sensor further comprises a continuous lactate sensor;
the set of analyte measurements comprises lactate measurements;
the true analyte levels of the user comprise lactate levels of the user; and
the one or more processors are further configured to execute the executable instructions to:
determine, based on the set of estimated analyte measurements, a lactate metric indicating a trend of the lactate levels of the user.

29. The monitoring system of claim 28, wherein the therapy management guidance is determined based on a first comparison of the one or more ammonia metrics to one or more ammonia thresholds and a second comparison of the lactate metric to one or more lactate thresholds of the user.

30. The monitoring system of claim 29, wherein:
the first comparison indicates that the ammonia levels of the user are increasing at a fast rate;
the second comparison indicates that the lactate levels of the user are increasing; and
the therapy management guidance comprises guidance relating to a presence or risk of an acute health event.

31. The monitoring system of claim 30, wherein the acute health even comprises variceal bleeding.

32. The monitoring system of claim 29, wherein:
the first comparison indicates that the ammonia levels of the user are increasing;
the second comparison indicates that the lactate levels of the user are increasing;
the increasing ammonia levels precede the increasing lactate levels; and
the therapy management guidance comprises guidance relating to a presence or risk of developing at least one of ascites, sepsis, or a bacterial positive infection.

33. The monitoring system of claim 1, wherein:
the continuous analyte sensor comprises a continuous glucose sensor;
the set of estimated analyte measurements comprises glucose measurements; and
the true analyte levels of the user comprise glucose levels of the user.

34. The monitoring system of claim 33, wherein the one or more processors are further configured to execute the executable instructions to:
determine, based on the set of estimated analyte measurements, one or more glucose metrics indicating a trend of the glucose levels of the user.

35. The monitoring system of claim 34, wherein the therapy management guidance is determined based on a first comparison of the one or more glucose metrics to one or more glucose thresholds of the user.

36. The monitoring system of claim 35, wherein:
the first comparison indicates that:
the glucose levels of the user are below a range normal for the user; and
the therapy management guidance comprises guidance related to a worsening liver function of the user.

37. The monitoring system of claim 36, wherein:
the continuous analyte sensor further comprises a continuous ammonia sensor;
the set of analyte measurements comprises ammonia measurements;
the true analyte levels of the user comprise ammonia levels of the user; and
the one or more processors are further configured to execute the executable instructions to:
determine, based on the set of estimated analyte measurements, an ammonia metric indicating a trend of the ammonia levels of the user.

38. The monitoring system of claim 37, wherein the therapy management guidance is determined based on the first comparison and the ammonia metric.

39. The monitoring system of claim 38, wherein:
the first comparison indicates that the glucose levels of the user are above a range normal for the user;
the ammonia metric indicates that the ammonia levels of the user are increasing; and
the therapy management guidance comprises guidance relating to at least one of a presence or risk of developing of developing hepatic encephalopathy or a worsening liver function of the user.

40. The monitoring system of claim 38, wherein:
the first comparison indicates that the glucose levels of the user are above a range normal for the user;
the ammonia metric indicates that the ammonia levels of the user are not increasing; and
the therapy management guidance comprises guidance relating to a worsening liver function of the user.
